(19) United States
(12) Patent Application Publication
(10) Pub. No.: US 2024/0285206 A1
(43) Pub. Date: Aug. 29, 2024

(54) Device and Method for Dynamic Determination of Medical Subtype, and Subtype Driven Personalized Medical Care

(71) Applicant: Sohyun Chung, FAIRFAX, VA (US)

(72) Inventor: Sohyun Chung, FAIRFAX, VA (US)

(73) Assignee: Sohyun Chung, FAIRFAX, VA (US)

(21) Appl. No.: 18/600,740

(22) Filed: Mar. 10, 2024

Related U.S. Application Data
(60) Provisional application No. 63/438,255, filed on Jan. 11, 2023.

(51) Int. Cl. A61B 5/16; A61B 5/00; A61B 5/291

(52) U.S. Cl. CPC A61B 5/165; A61B 5/291; A61B 5/4812; A61B 5/4839; A61B 5/7282; A61B 5/746

(57) ABSTRACT

Disclosed herein are devices and methods for dynamically determining the subtype of a medical disorder based on continuous monitoring of biometric data via a portable or wearable electronic device. Also disclosed herein are devices and methods of providing personalized medical care based on the dynamically determined subtype of a medical condition. The subtype can be dynamically updated based on the presentation of new symptoms, whereby the user can be provided with personalized medical care in a timely manner based on the updated subtype.

### CROSS-REFERENCE TO RELATED APPLICATIONS

This application claims the benefit of priority under 35 U.S.C. § 119(e) of U.S. Provisional Patent Application No. 63/438,255, filed Jan. 13, 2023, the entire disclosure of which is incorporated herein by reference in its entirety for all purposes.

### FIELD OF THE INVENTION

Disclosed herein are devices and methods for dynamically determining the subtype of a medical disorder based on continuous monitoring of biometric data via a portable or wearable electronic device. Also disclosed herein are devices and methods of providing personalized medical care based on the dynamically determined subtype of a medical condition. The subtype can be dynamically updated based on the presentation of new symptoms, whereby the user can be provided with personalized medical care in a timely manner based on the updated subtype.

### BACKGROUND OF THE INVENTION

Many psychiatric illnesses have a high chance of recurrence during the lifetime of patients even when put into a state of full remission. For example, bipolar disorder and schizophrenia have a very high chance of recurrence during lifetime. Thus, bipolar disorder and schizophrenia are chronic medical conditions that may impact a patient for years or decades.

Once a diagnosis of bipolar disorder is made, patient may be placed on a maintenance pharmaceutical treatment, and may experience several manic episodes and depressive episodes over their lifetime. The pattern and frequency of onset of manic episodes and depressive episodes may also change over time. If poorly managed, the patient's condition can deteriorate, and the patient can experience rapid cycling between depressive and manic episodes or become less responsive to treatment. These changes may correlate to biologic, cellular, and structural alternations to the patient's brain, making further treatment more difficult.

Likewise, psychotic disorders such as schizophrenia may involve multiple episodes. Due to the reduction in cognitive ability and inability to think clearly during episodes, it is difficult for patients to take their own medications or seek help during an active psychotic episode. Delay in treatment can result in permanent structural and neural network changes in the brain, which makes further treatment more difficult.

The treatment of bipolar disorder presents as a big challenge to clinicians due to the ability of a depressive episode to convert to a manic episode with the use of an antidepressant. The selective serotonin reuptake inhibitors (SSRI, e.g. fluoxetine, sertraline, citalopram, paroxetine) and selective noradrenaline reuptake inhibitor (SNRI, e.g. duloxetine, venlafaxine, desvenlafaxine) typically used effectively with unipolar depression have been discouraged in the treatment of bipolar disorder due to fear that the medication will trigger a manic episode. This leaves bipolar disorder patients in a depressive mood with very limited therapeutic options. For example, Lamictal (lamotrigine) has been proposed as a possible mood stabilizer with mild anti-depressive effect. However, a bipolar disorder patient who is placed on Lamictal may experience severe depression from time to time, and the depressive episode may last much longer than the manic episode. A typical manic patient experience three times longer depressive episodes compared to their intermittent manic episode over their lifetime. Medications offered for unipolar depression, such as fluoxetine is contraindicated due to the possibility of triggering a manic episode. Improved monitoring of patient's mood state is necessary to allow the patient to be treated according to his/her current mood state.

Likewise, psychiatrists and clinicians have been unsuccessful in continually monitoring psychotic patients and ensuring timely antipsychotic medication administration. Many patients with schizophrenia end up becoming jobless, homeless, with no social support due to their disease. Timely medication administration does not occur due to the inability of patients to care for oneself or to understand their clinical state.

The chronic nature of the disease also makes it difficult for clinicians to keep track of the entire disease course that a patient may have experienced over years or decades of their lives. When psychiatrists hold an interview with a bipolar disorder patient to determine the best therapy, patients often do not recall the detailed history of their disease or the medications that have and have not been efficacious for their disease. Manic episodes and psychotic episodes that a patient have experienced may feel like a hazy remote dream to a patient in full remission, who now possesses rational thinking capacity. The patient often cannot explain what has taken place during their unmonitored active episodes.

During an acute episode, the patient is often not aware that he/she is in an acute manic episode, a depressive state, having a delusion, or having a hallucination. The disease may cloud their judgement, compromise their cognitive abilities, reduce their insight regarding their disease, and/or cause an internal preoccupation that keeps the patient too busy to deal with his/her condition in an effective manner. Many bipolar patients have committed themselves to risky business projects or spent too much money during their manic episodes, only to recover from the manic episode and to enter a depressive episode with eroded self-confidence. Feeling less energy with negative outlook on life, less confident than the manic version of themselves, the patients often feel hopeless regarding the difficult situation that they are committed to. It would be helpful if patients can identify the onset of their manic episode, seek early treatment and be reminded not to engage in risk decision making during a manic episode.

Many psychotic patients have taken actions that were illogical or even harmful to themselves and others during their psychotic episode. An early detection of the onset of a new episode and alerting others for appropriate help can prevent patients from making grave mistakes that can negatively impact their lives in the long term.

### SUMMARY OF THE INVENTION

Disclosed herein are devices and methods for dynamically determining the subtype of a psychiatric disorder based on continuous monitoring of biometric data via a portable or wearable electronic device. Further disclosed herein are devices and methods of autonomically providing personalized medical care based on a dynamically determined subtype of a psychiatric disorder. The subtype can be dynamically updated based on the presentation of new symptoms, whereby the user can be provided with a personalized care in a timely manner based on the change in subtype.

Disclosed herein is a device for dynamically determining psychiatric disorder subtype and automatically providing intervention based on the subtype determination. The device may include: a user terminal including a processor and memory, wherein the user terminal is configured to receive user input from a user; a data collection unit configured to collect biometric data from a wearable device; an assessment unit is configured to: process the biometric data to determine an emotional state of the user and to determine a psychiatric disorder subtype and store the determined subtype in memory, dynamically update the stored subtype in response to receiving new biometric data that indicates either the stored subtype is incorrect or needs an additional qualifier; and initiate an alert for offering an intervention to the user.

The intervention manager may include a processor. The intervention manager may be configured to offer the intervention to the user in response to an alert from the assessment unit. The intervention manager may be configured to offer at least one event selected from the group consisting of: patient notification, healthcare provider notification, social support notification, suicide hotline connection, virtual therapy, music therapy, mindfulness therapy, light therapy, auditory hallucination avatar therapy, RX dispensing, biofeedback session, neurofeedback session, or a combination thereof.

The wearable device may be configured to be worn continuously by the user over an extended length of time, such as one week or longer, and the assessment unit may be configured to dynamically update the current subtype based on dynamically received information from the wearable device. Here, if the user is to continually wear the wearable device for two weeks, it is understood that the user may take off the wearable device at night or when taking a bath, for example. Even if the user is not 100% compliant with the use of a wearable device, the wearable device still makes it possible to obtain more data over time in comparison to the traditional intermittent visit to a doctor's office. The wearable device may be configured to be worn daily by the user over an intended length of time of one month or longer, and the assessment unit may be configured to dynamically update the current subtype based on dynamically received information from the wearable device. The wearable device may be configured to be worn daily by the user over an intended length of time of one year or longer, and the assessment unit may be configured to dynamically update the current subtype based on dynamically received information from the wearable device. The intervention manager may be triggered to offer an intervention in response to an updating of the current subtype stored in the assessment storage.

The wearable device may include an electrode to be mounted on the head of the user to collect brain wave related information. The brain wave information may be used to determine sleep architecture of the user, and a determination may be made regarding whether the user is developing a new onset of manic episode by comparing the average sleep length of the user in a range of time between one day to seven days to the average range of sleep of the user in a range of time between a month to twenty years.

The brain wave information may be used to determine sleep architecture of the user, and the sleep architecture may be used to determine whether the user has a reduced REM latency.

The user terminal may be configured to collect information regarding the user's daily mood, and the assessment unit may be configured to generate a mood chart over an extended time in a range of one week to one hundred years, and the assessment unit may be configured to determine whether the user is euthymic, hypomanic, manic, or depressed based on the mood chart. The mood chart may allow the device to determine whether the user is experiencing a rapid cycling of mood.

The device may be configured to alert at least one of the user, a healthcare provider of the user, a social support of the user, or a combination thereof, in response to a determination that the patient may be experiencing the rapid cycling, and the device may be configured to update the subtype to reflect the presence of the rapid cycling.

The device may be configured to alert at least one of the user, a healthcare provider of the user, a social support of the user, or a combination thereof, in response to a determination that the user is experiencing a new onset of manic episode.

The device of claim 11, wherein the device alerts the user of the determination that the user is experiencing the new onset of manic episode and warn the user to modify behavior, and the warning comprises at least one selected from the group consisting of getting more sleep, refraining from spending money, refraining from risky behavior, anger management, refraining from big life decisions, stop antidepressant, stop light therapy or modify light therapy, listen to inspirational message, contact social support, contact healthcare professional, take an antipsychotic medication, or a combination thereof.

The device may further include parenteral medication administerer, the administerer including a psychotropic medication storage, and configured to inject the user with a predetermined dose of medication as an intervention in response to a patient consent. The medication may be an antipsychotic, and the device may be configured to obtain an approval of the medication administration from the healthcare provider in advance. The mood chart may allow the device to determine whether the patient has entered a new depressive episode. The intervention manager may be configured to offer at least one event selected from the group consisting of music therapy, virtual therapy, mindfulness exercise, light therapy, or a combination thereof in response to the determination that the patient entered a depressive episode; and a biofeedback related information may be obtained via biosensors worn by the user while the event is offered to the user to determine an efficacy of the event on the user. The efficacy of the event may be used to update a list of effective intervention, such that an effective intervention may be preferentially offered to the user rather than an ineffective intervention. The subtype may include at least one selected from the group consisting of: bipolar I disorder, bipolar II disorder, unipolar depression, schizoaffective disorder, schizophrenia, schizophreniform disorder, brief psychosis, adjustment disorder, PTSD, or a combination thereof. At least one qualifier of the subtype may be selected from the group consisting of: with psychosis; without psychosis; with single episode; with repeated episodes; with recurrent disease; with rapid cycling; with paranoid delusion; without paranoid delusion; with ideas of reference; without ideas of reference; with hallucination; without hallucination; with auditory hallucination; with visual hallucination; without gustatory hallucination; with history of hospitalization; without history of hospitalization; with history of suicide attempt; without history of suicide attempt; with history of violent behavior; without history of violent behavior; with suicidal ideation; with homicidal ideation; with substance use; induced by substance; induced by another medical condition; induced by neurodegenerative disease; with abnormal brain structure; with abnormal brain function at corpus callosum; with abnormal white matter structure; with reduced gray matter; with abnormal brain wave; with abnormal sleep architecture; with normal sleep architecture; with reduced sleep at onset of manic phase; with seasonal affective disorder; with diurnal mood swing (AM or PM); with money spending issues during manic state; with risky business decision during manic state; with reckless activity during manic state; with grandiosity during manic state; with depression responsive to neuromodulation; with irritability during manic state; with substance abuse during manic state; with psychosis during manic state; with suicide attempt during depressive state; with psychosis during depressive state; with depression responsive to light therapy; with depression responsive to antidepressant; with depression responsive to mindfulness exercise; with depression responsive to ECT; with depression responsive to TMS; with psychosis responsive to antipsychotic; with genetic predisposition; with family history; with mild symptom; with moderate symptom; with severe symptom; post stroke; post traumatic brain injury; with concern for dementia; responsive to psychotherapy; responsive to antidepressant; responsive to antipsychotic; responsive to long-term injectable antipsychotic; with extra-pyramidal symptom; with history of tardive dyskinesia; with onset of tardive dyskinesia; responsive to anticholinergic agent; responsive to mood stabilizer; responsive to light therapy; responsive to biofeedback meditation; responsive to music therapy; responsive to neurofeedback meditation; responsive to extradoses of antipsychotic; responsive to sleep aid; responsive to ECT; responsive to TMS; currently in active manic episode; currently in depressive episode; currently experiencing psychosis; currently in euthymic mood; currently in depressive mood; currently in elevated mood; or a combination thereof.

Provided herein is a method for dynamically determining psychotic disorder subtype and providing intervention based on the subtype, the method including the steps of: receiving user input from a user via a user terminal including a processor and memory; collecting biometric data of the user via a wearable device; processing the biometric data on processor to determine psychotic disorder subtype and store the determined psychotic disorder subtype in memory; continually receiving biometric data from the wearable device over a prolonged length of time exceeding one week, and continually determining whether the psychotic disorder subtype is to be updated; and dynamically updating the psychotic disorder subtype based on the determination, and initiating an intervention based on the updated psychotic disorder subtype, wherein the psychotic disorder subtype comprises: a DSM diagnosis comprising at least one of: bipolar disorder, unipolar depression, schizoaffective disorder, schizophrenia, schizophreniform disorder, adjustment disorder, generalized anxiety disorder, OCD; and one or more qualifiers comprising at least one of: with psychosis; without psychosis; with single episode; with repeated episodes; with recurrent disease; with rapid cycling; with paranoid delusion; without paranoid delusion; with ideas of reference; without ideas of reference; with hallucination; without hallucination; with auditory hallucination; with visual hallucination; without gustatory hallucination; with history of hospitalization; without history of hospitalization; with history of suicide attempt; without history of suicide attempt; with history of violent behavior; without history of violent behavior; with suicidal ideation; with homicidal ideation; with substance use; induced by substance; induced by another medical condition; induced by neurodegenerative disease; with abnormal neuro structure; with abnormal neuro structure at corpus callosum function; with abnormal white matter structure; with reduced gray matter; with abnormal brain wave; with abnormal sleep architecture; with normal sleep architecture; with reduced sleep at onset of manic phase; with seasonal affective disorder; with diurnal mood swing (AM or PM); with money spending issues during manic state; with risky business decision during manic state; with reckless activity during manic state; with grandiosity during manic state; with depression responsive to neuromodulation; with irritability during manic state; with substance abuse during manic state; with psychosis during manic state; with suicide attempt during depressive state; with psychosis during depressive state; with depression responsive to light therapy; with depression responsive to antidepressant; with depression responsive to mindfulness exercise; with depression responsive to ECT; with depression responsive to TMS; with psychosis responsive to antipsychotic; with genetic predisposition; with family history; with mild symptom; with moderate symptom; with severe symptom; post stroke; post traumatic brain injury; with concern for dementia; responsive to psychotherapy; responsive to antidepressant; responsive to antipsychotic; responsive to long-term injectable antipsychotic; with extra-pyramidal symptom; with history of tardive dyskinesia; with onset of tardive dyskinesia; responsive to anticholinergic agent; responsive to mood stabilizer; responsive to light therapy; responsive to biofeedback meditation; responsive to music therapy; responsive to neurofeedback meditation; responsive to extradoses of antipsychotic; responsive to sleep aid; responsive to ECT; responsive to TMS; currently in active manic episode; currently in depressive episode; currently experiencing psychosis; currently in euthymic mood; currently in depressive mood; currently in elevated mood; or a combination thereof.

Provided herein is a device for dynamically determining bipolar disorder subtype and providing intervention based on the subtype, the device including a user terminal including a processor and memory, wherein the user terminal is configured to receive user input from a user; a data collection unit configured to collect biometric data from a wearable device; an assessment unit configured to: process the biometric data to determine an emotional state of the user and to determine a bipolar disorder subtype and store the determined subtype in memory, dynamically update the stored subtype in response to receiving new biometric data that indicates either the stored subtype is incorrect or needs an additional qualifier; and initiate an alert for offering an intervention to the user; and an intervention manager comprising a processor, the intervention manager configured to offer the intervention to the user in response to the alert from the assessment unit, wherein the intervention includes at least one event selected from the group consisting of patient notification, healthcare provider notification, social support notification, suicide hotline connection, virtual therapy, music therapy, mindfulness therapy, light therapy, auditory hallucination avatar therapy, RX dispensing, biofeedback session, neurofeedback session, or a combination thereof.

Provided herein is a method of determining efficacious intervention, the method including: collecting biometric data of the user via a wearable device; processing the biometric data on processor to determine psychotic disorder subtype and store the determined psychotic disorder subtype in memory; dynamically receiving new biometric data from the wearable device over a prolonged length of time exceeding one week; dynamically initiating an intervention in response to an updating event of the stored psychotic disorder subtype; and receiving biometric data during the intervention and determining efficacy of the intervention based on the biometric data.

Provided herein is a method of elucidating correlation between endophenotypes and intervention efficacy, the method including the steps of: collecting biometric data of multiple users via wearable devices; collecting endophenotype data of the multiple users; determining psychotic disorder subtype of each of the multiple users, wherein the biometric data are collected over a prolonged length of time to exceed 1 months; and tracking efficacy of pharmaceutical agents used by each users; and assigning positive points for efficacious intervention and negative points for failed or non-efficacious intervention for each of the pharmaceutical agents.

Provided herein is a method of determining correlation between an intervention and a subtype, the method including the steps of: collecting biometric data of multiple users via wearable devices; collecting endophenotype data of the multiple users; determining psychotic disorder subtype of each of the multiple users, wherein the biometric data are collected over a prolonged length of time to exceed 1 months; and tracking efficacy of pharmaceutical agents used by each users; and assigning points corresponding to correlation of the endophenotypes and the psychotic disorder subtype.

Provided herein is a method of re-classifying psychiatric disorders, the method including: collecting biometric data of multiple users via wearable devices over prolonged length of time, the prolonged length of time comprising: one day, one week, one month, one year, 5 years, and/or 10 years or longer; determining phenotype data of the multiple users over prolonged time, wherein the phenotype data includes: the presence or the absence of a symptom, the symptom comprising at least one elected from the group consisting of depression, mania, hypomania, psychosis, delusion, hallucination, or a combination thereof, wherein at least some of the users interaction with an AI character projected on a touchscreen of an electronic device to answer questions regarding the users' phenotype; collecting endophenotype data of the multiple users over prolonged time, wherein the endophenotype data includes: the presence or the absence of an endophenotype, the symptom including at least one elected from the group consisting of sleep architecture abnormality, sleep length abnormality, seasonal affective disorder, dysregulation of motivation and reward, dysregulation of emotional reactivity, impaired facial expression recognition, attention and concentration dysfunction, executive dysfunction, impulsivity dysfunction, and suicidality or suicidal ideation, or a combination thereof; collecting brain structural data of the multiple users, the brain structural data including: the presence or the absence of white matter abnormalities, anterior cingulate cortex abnormalities, volume reduction in the anterior cingulate cortex, volume reduction in the anterior cingulate cortex ventral and anterior to the genu of corpus callosum, volume reduction in left subgenual anterior cingulate cortex, brain connectivity abnormality (such as detected by fMRI or functional medical imaging), brain structural abnormality (such as detected by MRI, CT or other structural medical imaging), or a combination thereof; or collecting brain functional data of the multiple users, the brain functional data (such as detected by fMRI such as BOLD or ReHo, PET or other functional medical imaging) including: the presence or the absence of hypoactive brain area, hyperactive brain area, hypoactive brain connectivity, hyperactive brain connectivity, whole brain decreased connectivity, decreased ReHo in frontal lobe, increased parahippocampal activation, decreased ReHo in parietal lobe, decreased activation in anterior cingulate gyri, decreased activation of posterior cingulate gyri, decreased activation of occipital lobe, increased activation of thalamus, increased activation of hypothalamus, decreased activation of cerebellum, increased activation of striatum, altered activity of medial frontal and anterior cingulate gyri, decreased fronto-temporal white matter functional activation, or a combination thereof; collecting neuroinflammatory marker data of the multiple users, the neuroinflammatory marker data including: the presence or absence of abnormalities in neuroinflammatory markers or an elevation or a reduction in the level of neuroinflammatory markers, the neuroinflammatory markers comprising at least one of IL-10, IL-6, TNF, cytokines, osteoproteigerin (OPG), C-reactive protein (CRP) or a combination thereof, in serum, plasma, and/or CSF; collecting macromolecule and gene products data of the multiple users, the macromolecule and gene products data includes: the presence or absence of abnormalities in cerebral glutamate level, CCL11, sTNFR1, CCL24, CXCL10, BDNF, CRP, TWEAK, IL-10, OPG, C-reactive protein (CRP) and a combination thereof; collecting EEG signal data of the multiple users, wherein EEG signal data comprises the presence or absence in abnormalities in sleep architecture, reduction in REM latency, pattern deviation from baseline pattern taken while patient was awake and balanced, or a combination thereof; collecting genetic data from the multiple users, wherein the genetic data includes: brain connectivity abnormality (such as detected by fMRI or functional medical imaging), brain structural abnormality (such as detected by MRI, CT or other structural medical imaging), abnormal proteins, cerebral glutamate level, abnormal EEG signal; collecting clinical treatment responsiveness data from the multiple users, wherein clinical treatment responsiveness data includes: the responsiveness or lack of responsiveness of: antidepressant (SSRI, SNRI), antipsychotic (first generation antipsychotic, second generation antipsychotic), mood stabilizer (lithium, second generation antipsychotic), anticholinergic agents (benztropine, Benadryl), sleep aid (melatonin, trazodone), or a combination thereof; collecting intervention efficacy data from the multiple users, wherein intervention efficacy data includes: the efficacy or lack of efficacy of: light lamp therapy for seasonal affective disorder, music therapy, biofeedback therapy, neurofeedback therapy, virtual avatar counseling, psychosis voice hallucination avatar training, mindfulness exercise, or a combination thereof; collecting current symptom data from the multiple users, wherein the current symptom data includes: the presence or absence of: current manic episode, current depressive episode, current suicidal ideation, current suicidal plan, current homicidal ideation, current agitation, current irritability, current violence potential, current risk for elopement, current code strong, current hospitalization, current psychosis, current paranoia, current idea of reference, current auditory hallucination, current delusion, current extra-pyramidal symptoms, current tardive dyskinesia, or a combination thereof; determining the psychiatric disorder subtype of each of the multiple users, wherein the subtype is stored in a tree data structure indicating the presence or the absence, the responsiveness or lack of responsiveness, or the efficacy and a lack of efficacy, for at least one of: a symptom, a phenotype data, an endophenotype data, a brain structural data, a brain functional data, neuroinflammatory marker data, macromolecule and gene products data, pharmaceutical agents or other interventions, efficacy of an intervention on a specific user, and/or current symptom data; and determining association or correlation between pharmaceutical agents and the psychiatric disorder subtype of each of the multiple users.

Provided herein is an automatic medication dispenser, the dispenser including: a housing configured to be wearable on the user; an autoinjector disposed inside the housing and configured to hold a medication; and an antiseptic pad disposed on a tip of the autoinjector to sterilize an injection site; and an actuator configured to administer the medication inside the autoinjector after the injection site has been sterilized by the antiseptic pad. The dispenser may be configured to be mounted over a deltoid muscle of the user; the autoinjector is configured to hold a preloaded syringe containing a long acting injectable medication; the antiseptic pad is disposed on a tip of the autoinjector such that a pivoting of the autoinjector from a horizontal position to a vertical position above the skin is configured to cause the antiseptic pad to wipe the injection site and sterilize the injection site; and the actuator is configured to automatically inject the medication on the deltoid muscle upon actuated by either a manual push or an electronic signal from a remote device, wherein the remote device is a user terminal or a healthcare professional terminal.

Two or more autoinjectors may be disposed within the housing such that different medication may be dispensed at different time, and wherein the automatic medication dispenser is configured to draw a small amount of blood from the user for blood work, including at least one selected from the group consisting of: blood glucose, lipid panel, triglyceride level, LDH, HDL, cholesterol, liver enzymes, hemoglobin A1 C, basic metabolic panel (BMP), complete blood count (CBC), white blood count, alkaline phosphatase (ALP), alanine transaminase (ALT), aspartate aminotransferase (AST), gamma-glutamyl transferase (GGT), Depakote level, lithium level, lamotrigine level, serum toxicology, and a combination thereof.

Disclosed herein is a device for dynamically determining medical subtype and providing intervention based on the subtype, the device including: a user terminal comprising a processor and memory, wherein the user terminal is configured to receive user input; a data collection unit configured to collect biometric data from a wearable device and store the collected biometric data in a data collection storage; an assessment unit configured to process the biometric data to determine presence of a symptom in a user and to determine medical subtype and store the determined medical subtype in an assessment storage, wherein the assessment unit includes: a dynamic subtype update unit that is configured to dynamically update the current medical subtype in response to receiving new biometric data that indicates either the stored subtype is incorrect or needs additional qualifier; and an alert initiator that is configured to determine timing of offering an intervention to the user; and an intervention manager is configured to offer the intervention to the user in response to the determining of the alert initiator. The intervention manager may be configured to offer at least one event from the group consisting of patient notification, healthcare provider notification, social support notification, suicide hotline connection, virtual therapy, music therapy, mindfulness therapy, light therapy, RX dispensing, biofeedback session, neurofeedback session, or a combination thereof.

The medical subtype may be a psychiatric disorder subtype, and the assessment unit may be configured to process the to process the biometric data to determine an emotional state of a user and to determine the psychiatric disorder subtype and store the determined psychiatric disorder subtype in an assessment storage. The assessment unit may include a dynamic subtype update unit that is configured to dynamically update the current affect disorder subtype in response to receiving new biometric data that indicates either the stored subtype is incorrect or needs additional qualifier; and an alert initiator that is configured to determine timing of offering an intervention to the user; and an intervention manager is configured to offer the intervention to the user in response to the determining of the alert initiator. The intervention manager may be configured to offer at least one event from the group consisting of patient notification, healthcare provider notification, social support notification, suicide hotline connection, virtual therapy, music therapy, mindfulness therapy, light therapy, RX dispensing, biofeedback session, neurofeedback session, or a combination thereof.

The medical subtype may be a neurological disease subtype, such as seizure disorder. The assessment unit may be configured to process the to process the biometric data to determine a seizure state of a user and to determine the neurological disease subtype and store the determined neurological disease subtype in an assessment storage. The subtype may specify any genetic cause for the neurological disease, trigger for the seizure such as sleep deprivation, flashing light, seasonal change, fatigue, blood alcohol level, blood seizure medication level, and time of day. The data collection unit may be able to collect when seizure medications are taken, the doses of medication task, the blood level of the medication, etc. Intervention manager may be able to determine when the patient may develop seizure, and engage patient alert initiator, healthcare provider alert, social support alert, and the like.

The medical subtype may be a neurodegenerative disease subtype, and the assessment unit may be configured to process the to process the biometric data to determine a cognitive ability of a user and to determine the neurodegenerative disease subtype and store the determined neurodegenerative disease subtype in an assessment storage.

The medical subtype may be a neurotoxicological disease subtype, and the assessment unit may be configured to process the to process the biometric data to determine a cognitive ability or a motor ability of a user and to determine the neurotoxicological disease subtype and store the determined neurotoxicological disease subtype in an assessment storage. For example, the neurotoxicological disease may be neurotoxicity due to manganese, tremors due to manganese, neurogenerative disease caused by manganese, cognitive issues caused by lead exposure, etc. Urine heavy metal test, blood heavy metal test, bone x-ray lead exam score, MRI T1 signal intensity in globus pallidus, and other heavy metal test results may be collected via the data collection unit. Subjective mood may be collected by the collection unit to assess the mood.

The medical subtype is a cardiovascular condition subtype, and the assessment unit may be configured to process the biometric data to determine a heart rate, heart rhythm, EKG, or physical activity level of a user and to determine the cardiovascular condition subtype and store the determined subtype subtype in an assessment storage. For example, the cardiovascular condition subtype may be atrial fibrillation, cardiac arrythmia, congestive heart failure, with specific metabolic equivalent of task (MET) that the user can perform, frequency of A-fib or other cardiac arrythmia, specifics regarding when the arrythmia is likely to occur based on past data such as exertional cause, temperature related cause, emotional response, etc.

The wearable device may be configured to be worn continuously by the user over an intended length of time of one week or longer, and the assessment unit may be configured to dynamically update the current subtype based on dynamically received information from the wearable device.

The wearable device may be configured to be worn daily by the user over an intended length of time of one month or longer, and the assessment unit may be configured to dynamically update the current subtype based on dynamically received information from the wearable device.

The wearable device may be configured to be worn daily by the user over an intended length of time of one year or longer, and the assessment unit may be configured to dynamically update the current subtype based on dynamically received information from the wearable device.

The intervention manager may be triggered to offer an intervention in response to an updating of the current subtype stored in the assessment storage.

The wearable device may include an electrode to be mounted on the head of the user to collect brain wave related information. The brain wave information may be used to determine sleep architecture of the user, and a determination may be made regarding whether the user is developing a new onset of manic episode by comparing the average sleep length of the user in a range of time between one day to seven days to the average range of sleep of the user in a range of time between a month to twenty years.

Disclosed herein is a device for dynamically determining bipolar disorder subtype and providing intervention based on the subtype, the device including: a user terminal comprising a processor and memory. The user terminal may be configured to receive user input from a user. The device may include a data collection unit configured to collect biometric data from a wearable device; an assessment unit configured to: process the biometric data to determine an emotional state of the user and to determine a bipolar disorder subtype and store the determined subtype in memory, dynamically update the stored subtype in response to receiving new biometric data that indicates either the stored subtype is incorrect or needs an additional qualifier; and initiate an alert for offering an intervention to the user; and an intervention manager comprising a processor, the intervention manager configured to offer the intervention to the user in response to the alert from the assessment unit. The intervention may include at least one event selected from the group consisting of patient notification, healthcare provider notification, social support notification, suicide hotline connection, virtual therapy, music therapy, mindfulness therapy, light therapy, auditory hallucination avatar therapy, RX dispensing, biofeedback session, neurofeedback session, or a combination thereof.

Disclosed herein is a method of determining efficacious intervention, the method including: collecting biometric data of the user via a wearable device; processing the biometric data on processor to determine psychotic disorder subtype and store the determined psychotic disorder subtype in memory; dynamically receiving new biometric data from the wearable device over a prolonged length of time exceeding one week; dynamically initiating an intervention in response to an updating event of the stored psychotic disorder subtype; and receiving biometric data during the intervention and determining efficacy of the intervention based on the biometric data.

Disclosed herein is a method of elucidating correlation between endophenotypes and intervention efficacy, the method including: collecting biometric data of multiple users via wearable devices; collecting endophenotype data of the multiple users; determining psychotic disorder subtype of each of the multiple users, wherein the biometric data are collected over a prolonged length of time to exceed 1 months; and tracking efficacy of pharmaceutical agents used by each user; and assigning positive points for efficacious intervention and negative points for failed or non-efficacious intervention for each of the pharmaceutical agents.

Disclosed herein is a method of determining correlation between an intervention and a subtype, the method including: collecting biometric data of multiple users via wearable devices; collecting endophenotype data of the multiple users; determining psychotic disorder subtype of each of the multiple users, wherein the biometric data are collected over a prolonged length of time to exceed 1 months; and tracking efficacy of pharmaceutical agents used by each user; and assigning points corresponding to correlation of the endophenotypes and the psychotic disorder subtype.

Disclosed herein is a method of re-classifying psychiatric disorders, the method including: collecting biometric data of multiple users via wearable devices over prolonged length of time, the prolonged length of time comprising: one day, one week, one month, one year, 5 years, and/or 10 years or longer; determining phenotype data of the multiple users over prolonged time. The phenotype data may include the presence or the absence of a symptom, the symptom including at least one elected from the group consisting of depression, mania, hypomania, psychosis, delusion, hallucination, or a combination thereof, wherein at least some of the users interaction with an AI character projected on a touchscreen of an electronic device to answer questions regarding the users' phenotype; collecting endophenotype data of the multiple users over prolonged time. The endophenotype data may include the presence or the absence of an endophenotype, the symptom comprising at least one elected from the group consisting of sleep architecture abnormality, sleep length abnormality, seasonal affective disorder, dysregulation of motivation and reward, dysregulation of emotional reactivity, impaired facial expression recognition, attention and concentration dysfunction, executive dysfunction, impulsivity dysfunction, and suicidality or suicidal ideation, or a combination thereof; collecting brain structural data of the multiple users, the brain structural data may include: the presence or the absence of white matter abnormalities, anterior cingulate cortex abnormalities, volume reduction in the anterior cingulate cortex, volume reduction in the anterior cingulate cortex ventral and anterior to the genu of corpus callosum, volume reduction in left subgenual anterior cingulate cortex, brain connectivity abnormality (such as detected by fMRI or functional medical imaging), brain structural abnormality (such as detected by MRI, CT or other structural medical imaging), or a combination thereof; or collecting brain functional data of the multiple users, the brain functional data (such as detected by fMRI such as BOLD or ReHo, PET or other functional medical imaging) including: the presence or the absence of hypoactive brain area, hyperactive brain area, hypoactive brain connectivity, hyperactive brain connectivity, whole brain decreased connectivity, decreased ReHo in frontal lobe, increased parahippocampal activation, decreased ReHo in parietal lobe, decreased activation in anterior cingulate gyri, decreased activation of posterior cingulate gyri, decreased activation of occipital lobe, increased activation of thalamus, increased activation of hypothalamus, decreased activation of cerebellum, increased activation of striatum, altered activity of medial frontal and anterior cingulate gyri, decreased fronto-temporal white matter functional activation, or a combination thereof; collecting neuroinflammatory marker data of the multiple users, the neuroinflammatory marker data comprises: the presence or absence of abnormalities in neuroinflammatory markers or an elevation or a reduction in the level of neuroinflammatory markers, the neuroinflammatory markers comprising at least one of IL-10, IL-6, TNF, cytokines, osteoproteigerin (OPG), C-reactive protein (CRP) or a combination thereof, in serum, plasma, and/or CSF; collecting macromolecule and gene products data of the multiple users, the macromolecule and gene products data including: the presence or absence of abnormalities in cerebral glutamate level, CCL11, sTNFR1, CCL24, CXCL10, BDNF, CRP, TWEAK, IL-10, OPG, C-reactive protein (CRP) and a combination thereof; collecting EEG signal data of the multiple users, wherein EEG signal data comprises the presence or absence in abnormalities in sleep architecture, reduction in REM latency, pattern deviation from baseline pattern taken while patient was awake and balanced, or a combination ta collecting genetic data from the multiple users, wherein the genetic data comprises: brain connectivity abnormality (such as detected by fMRI or functional medical imaging), brain structural abnormality (such as detected by MRI, CT or other structural medical imaging), abnormal proteins, cerebral glutamate level, abnormal EEG signal; collecting clinical treatment responsiveness data from the multiple users, wherein clinical treatment responsiveness data including: the responsiveness or lack of responsiveness of: antidepressant (SSRI, SNRI), antipsychotic (first generation antipsychotic, second generation antipsychotic), mood stabilizer (lithium, second generation antipsychotic), anticholinergic agents (benztropine, Benadryl), sleep aid (melatonin, trazodone), or a combination thereof; collecting intervention efficacy data from the multiple users, wherein intervention efficacy data including: the efficacy or lack of efficacy of: light lamp therapy for seasonal affective disorder, music therapy, biofeedback therapy, neurofeedback therapy, virtual avatar counseling, psychosis voice hallucination avatar training, mindfulness exercise, or a combination thereof. collecting current symptom data from the multiple users, wherein the current symptom data may include: the presence or absence of: current manic episode, current depressive episode, current suicidal ideation, current suicidal plan, current homicidal ideation, current agitation, current irritability, current violence potential, current risk for elopement, current code strong, current hospitalization, current psychosis, current paranoia, current idea of reference, current auditory hallucination, current delusion, current extra-pyramidal symptoms, current tardive dyskinesia, or a combination thereof; determining the psychotic disorder subtype of each of the multiple users, wherein thesubtype is stored in a binary tree structure indicating the presence or the absence, the responsiveness or lack of responsiveness, or the efficacy and a lack of efficacy, for at least one of: a symptom, a phenotype data, an endophenotype data, a brain structural data, a brain functional data, neuroinflammatory marker data, macromolecule and gene products data, pharmaceutical agents, other interventions, and/or current symptom data; and determining association or correlation between pharmaceutical agents and the psychiatric disorder subtype of each of the multiple users.

Provided herein is a device for transmitting a medical subtype to a remote device, the device including: a user terminal including a processor and memory, wherein the user terminal is configured to receive user input from a user; a data collection unit configured to collect biometric data from a wearable device; an assessment unit configured to: process the biometric data to determine a medical subtype and store the determined medical subtype in memory, dynamically update the stored medical subtype in response to receiving new biometric data that indicates either the stored medical subtype is incorrect or needs an additional qualifier; and a medical subtype token generator that prepares a data packet that includes a representation for the stored medical subtype for transfer to the remote device; and a medical subtype visual token renderer that prepares a visual image as a representation for the stored medical subtype for transfer to the remote device as medical subtype visual token (MSVT).

The device may further include a camera configured to capture an image of a medical subtype visual token displaced on a screen of a remote device or printed-out on a paper; a medical subtype token interpreter configured to convert the image of the medical subtype visual token into a data structure that stores the medical subtype; a wired or wireless port or a blue tooth port that receives a data packet that includes a representation of a medical subtype from a remote device.

The device may further include: an intervention manager including a processor, the intervention manager configured to offer the intervention to the user in response to the alert from the assessment unit. The intervention may include at least one event selected from the group consisting of patient notification, healthcare provider notification, social support notification, suicide hotline connection, virtual therapy, music therapy, mindfulness therapy, light therapy, auditory hallucination avatar therapy, RX dispensing, biofeedback session, neurofeedback session, call an ambulance, or a combination thereof.

The device may further include a screen configured to display the visual image of the medical subtype visual token in order to allow a camera installed or connected to the remote device to scan the medical subtype visual token to transfer the subtype information. The device may further include a data transmitter configured to transmit the medical subtype token to the remote device via Internet, Bluetooth, hard wire, or a memory key. The medical subtype visual token may include time of generation information, and ICD-10 or DMS-5 classes as a visual representation. The medical subtype visual token may be a rendering of a 3D polygon that may be rotated to generate a 4 D video image captured by the camera of another device.

### BRIEF DESCRIPTION OF DRAWINGS

Embodiments of the present disclosure will now be described, by way of examples only, with reference to the attached Figures, wherein:

FIG. 1 depicts an example of a device that dynamically determines the subtype of a psychiatric disorder and provides timely personalized medical care based on the dynamically determined subtype.

FIG. 2 depicts a chart showing examples of brain abnormalities found in different subtypes of psychiatric disorders.

FIG. 3 depicts an example of the international 10-20 EEG electrode placement.

FIG. 4A depicts an example of a bispectral index (BIS) strip.

FIGS. 4B and 4C depict examples of wearable EEG helmets.

FIG. 5A depicts an example of a normal sleep architecture as determined by brain wave pattern.

FIG. 5B depicts examples of EEG signals generated during various stages of sleep.

FIG. 6 depicts a schematic diagram explaining an example of a method of providing neurofeedback training.

FIGS. 7A and 7B depict examples of questionnaires that can be used to clarify the subtype of a patient.

FIG. 8 depicts an example of graphical user interface that may be used to obtain subjective mood of a user.

FIGS. 9A to 9L depict examples of mood charts obtained from different users, the mood chart containing data collected over a various length of time.

FIGS. 10A and 10B depict examples of subtype data structures.

FIG. 11A depicts an example of a method of determining whether an intervention of interest can improve symptoms for a patient having a specific subtype.

FIG. 11B depicts an example of a method of providing personalized medical care.

FIG. 12 depicts an example of a method of classifying psychiatric disorders into subtypes.

FIGS. 13A, 13B, 13C, and 13D depict an example of a parenteral medication administerer 910.

FIG. 14 depicts an example of a method of determining the subtype of the psychiatric disorder of a patient.

FIGS. 15A, 15B, 15C, 15D and 15E depict examples of medical subtype visual tokens.

### DETAILED DESCRIPTION OF THE INVENTION

The present disclosure relates to devices and methods for dynamically determining the subtype of a psychiatric disorder based on continuous monitoring of biometric data obtained via a portable or wearable electronic device. The invention also relates to devices and methods of providing personalized medical care based on a dynamically determined subtype of a psychiatric disorder of a user. The subtype can be dynamically updated in response to the presentation of new symptoms, whereby the user can be provided with a personalized medical care in a timely manner.

The following description provides exemplary embodiment(s) only, and is not intended to limit the scope, applicability, or configuration of the disclosure. Rather, the following description of the exemplary embodiment(s) will enable those skilled in the art to implement various embodiments. It being understood that various changes may be made in the function and arrangement of elements without departing from the spirit and scope as set forth in the appended claims.

A “psychiatric disorder” as used herein and throughout this disclosure refers to mood disorders, major depression, bipolar disorder, unipolar depression, bipolar type I, bipolar type II, anxiety disorders, psychotic disorders, personality disorders, dementia, autism, behavioral and emotional disorders, delusions, substance use disorder, personality disorder, borderline personality disorder, narcissistic personality disorder, trauma-related disorder, Post-traumatic stress disorder, and the like. Mood disorders include bipolar I disorder, bipolar II disorder, unipolar depression, dysthymia, cyclothymia, mood disorder due to another medical condition, substance-induced mood disorder, schizoaffective disorder, and the like. Mood disorder due to another medical condition may include depression that presents in elderly due to a neurogenerative process or depression resulting from strokes to specific parts of the brain, from manganism, from chemical or oxidative stress to brain regions, post Covid-19 syndrome, for example. Psychotic disorders include schizophrenia, schizoaffective disorder, schizophreniform disorder, brief psychotic disorder, delusional disorder, psychotic disorder due to another medical condition, substance-induced psychotic disorder, medication-induced psychotic disorder, and paraphrenia. Substance-induced disorder may include hallucination due to alcohol use or alcohol withdrawal. Medication included psychotic disorder may include psychosis resulting from steroid use or ketamine abuse. Psychosis due to another medical condition may refer to visual hallucination present in dementia and other neurodegenerative disease. Schizophrenia may be further categorized into paranoid schizophrenia, hebephrenic schizophrenia, catatonic schizophrenia, undifferentiated schizophrenia, residual schizophrenia, simple schizophrenia, and unspecified schizophrenia. Anxiety disorders include social anxiety disorder, panic disorder, generalized anxiety disorder, agoraphobia, specific phobias, obsessive-compulsive disorder (OCD), selective mutism, separation anxiety disorder. Social anxiety disorder has the highest prevalence in general population, and some patients with phobia also exhibits panic. Trauma-related or stress-related disorders include PTSD, acute stress disorder, and adjustment disorder. Diagnostic and Statical Manual of Mental Disorders (DSM-5) provides additional diagnostic criteria for these conditions, which are incorporated herein. International Classification of Diseases (e.g. ICD-10, ICD-11) provide additional diagnostic criteria for psychiatric and neuropsychiatric conditions.

A “processor” or a “processing unit” as used herein and throughout this disclosure refers to a hardware electrical component, such as an electronic circuit, that performs an algorithm or a set of instructions. It may include multiple logic gates, arithmetic logic unit, combination logic, main memory, IO, a graphic processing unit, an artificial intelligence engine, or the like. A processor or a processing unit may be a microprocessor, microcontroller, embedded processor, digital signal processor and the like.

Modern processors have standardized 32- or 64-bit operations. These processors are often limited to operating on binary representations of data. However, the processors may use parallelization to increase throughput and additional commands to aggregate multiple binary operators. Modern microprocessors, such as a CPU, are implemented in conjunction with accelerated processing units such as a graphics processing unit (GPU), digital signal processor (DSP), field-programmable gate array (FPGA), or any other microprocessor specifically designed to accelerate one or more types of computations. A smartphone may include a mobile CPU as its processor. Processors have hardware component, such as circuits.

A “wearable device” as used herein and throughout this disclosure refers to a device that is designed to be wearable on the body, on top of clothes of a user, or applied on the skin of a user. For example, a wearable device may include an electronic device that is fastened to a user by a band, strap, or other form of attachment. Such a wearable device incorporates miniaturization and weight lightening technologies. A wearable device may be small, light, and highly portable. Because of the portability, a user may be able to use the device at the user's convenience in a mobile environment. Some wearable devices may be continuously worn by the user while engaging in daily activities, including when the user is participating in a sport, going to sleep, taking a road trip, driving, or talking with a friend, for example. A wearable device may automatically extract biometric information without the conscious engagement of the user. Various types of wearable devices, such as a wearable device for clothes and a wearable device for accessories, for example, may be used. In these contexts, the wearable device may be contained in or attached to the clothes or the accessories. Wearable devices include smart watch, wrist band, google glasses, electronic headband, wearable EEG strip, wearable EKG, accelerometer of smart phone, microphone, camera, biosensor electrodes and patches, implanted neuromodulation device, and other electronic devices or sensors which may be mounted on the body, glued, or applied to the skin of the user.

Bipolar disorder, schizophrenia, other affect disorders, schizophreniform disorders, and neuropsychiatric disorders resulting from an insult to the brain, such as stroke and manganism, may turn out to be a life-long condition that requires years or decades of monitoring and treatment since the initial onset. Medical care is often too expensive in terms of financial investment and time investment on the part of the patients, and thus it is not easy for patients to receive timely care for their chronic mental health disorder. For example, a patient with bipolar disorder may be placed on a maintenance dose of Depakote as mood stabilizer with appointments with psychiatrist scheduled every three months. If the patient starts having a manic episode one month after meeting with the psychiatrist, the psychiatrist may not be available to evaluate the patient or intervene on the behalf of the patient at the onset of the manic episode. During the manic episode, the patient may make poor life decisions that may have long-term negative impact on his/her life. Even their family members may suffer from negative impacts of the poor decisions undertaken by the patient during a manic episode, eroding on trust, and even leading to relationship problems, divorce, termination from employment, interaction with law enforcement, trauma, injury, and/or a downward socioeconomic spiral, with worsening self-esteem and depression. Accurate monitoring of emotion and revision of treatment plans between appointments with the psychiatrist will improve patient care. However, very few people have the financial resource and time resource to afford daily appointments with a psychiatrist.

Patients with schizophrenia may experience multiple psychotic episodes during their lifetime, and it is not easy for patients to respond to their worsening condition during an active psychotic episode because of delusional beliefs reduce their insight and judgement and hallucinations may affect their cognitive abilities. During an active psychotic episode, patients often forget to take their medication and do not contact their healthcare providers (physicians, therapist, nurses, allied health professionals) or their social support such as their family, caretakers, social worker, guardian ad litem, legal proxy, or healthcare power of attorney.

Continuous acquisition of biometric data is now possible through smart watches and other wearable devices without the user being consciously engaged with the data acquisition. A wearable device may include various sensors that gathers information, such as chemical sensors, electromechanical sensors, optical sensors, electrical sensors, microphone, camera, electrodes and the like. Examples of electromechanical sensors include accelerometer to measure movements, velocity, and angular rotation.

Optical sensors include sensors that may emit light into the body and measure reflected light and what is absorbed to detect various biological information such as heart rate, oxygen saturation, blood pressure. Electrical sensors are used for EEG, EKG, EMG and other electrical activity monitoring. Electrode dermal patches can be used to monitor sweat levels and perspiration. Chemical sensors can detect chemical concentration in sweat or other body fluid. For example, glucose monitoring may be performed by a chemical sensor applied to the skin of patient. Microphone of a wearable device can detect the voice of the user and even provide information regarding the user's contemporaneous environment and activity. A camera can capture the facial expression of the user. With the aid of AI software, it is now possible to determine the emotion portrayed by the facial expression. The GPS receiver in a smart phone can detect the location of a user. Other sensors, such as a digital stethoscope or an ultrasound, are now portable. The collected biometric data includes valuable information which provides a glimpse into the mental state, physical health, physical location, and activity levels of patients. By collecting the biometric data over an extended time, such as more than 1 month, 6 months, 1 year, or 5 years or more, the biometric data can be used in an algorithm to provide a high-level insight that can categorize the patient's mental health disease into very specific subtypes. The categorization can be used to dynamically devise a short-term treatment plan or intervention for the patient in between patient's appointments with his/her healthcare provider. In other words, a continuous and personalized medical care can be provided automatically without the constant involvement of a human healthcare provider on a day-to-day basis. This automatic care may be selected according to an algorithm preset by a clinician, hospital, clinic, or health management organization. The algorithm can be preset by the device manufacturer as a default setting and/or updated by a device management system protocol that is managed or updated by a monitoring center, a data clearing house, a population database study center, or by the patient, healthcare provider, or social support of the patient.

Further, a continued use of the device over months and years may allow several healthcare professionals and the patient to learn more about the individual patient's condition and learn about treatments and interventions that are efficacious for the individual patient. For example, a biofeedback mechanism can be employed to track the patient's response to an intervention or treatment. The device can also map the progression of the disease in a graphical manner over time. The progression of the disease can be tracked simultaneous to the time when the intervention or treatment is being implemented. By tracking the progression of the disease over a predetermined time range following offering an intervention or treatment, the device 10 can detect whether the intervention or treatment has been effective in dealing with the condition in the specific patient. The predetermined time range may comprise of minutes (1 minute to 60 minutes), hours (1 hour to 24 hours), days (1 day to 365 days), weeks (1 week to 52 weeks), months (1 month=30 days to 12 months), and/or years (1 year to 5 years, 1 year to 10 years, 1 year to 20 years, 1 year to lifetime of a patient). For example, the efficacy of a new pharmacological agent can be tracked over weeks to several months, and any improvement in symptoms can be alerted to the patient and healthcare provider in an automatically generated report.

Herein, “dynamic” manner refers to dynamic data processing. Dynamic data processing regards the processing of information that is periodically updated, meaning it changes asynchronously over time as new information becomes available. Data that is not dynamic is “static” or “persistent.” Such data is not likely to be modified and does not require frequent retrieval. Instead, in a dynamic data processing, new information is intermittently received. For example, new information regarding sleep deprivation may become available via wearable sensors and portable devices carried by the user. In dynamic process, the new information is timely processed to perform an algorithm or command. The information regarding sleep deprivation may raise concern over the development of a manic episode. The receipt of new information may trigger the assessment unit 300 to autonomically assess the clinical value of the new information.

When the processed new data includes clinically significant information, an action can be automatically triggered. Based on the triggering event, the device can update the subtype, provide timely alerts, implement an intervention, or obtain help for the user. For example, the sleep deprivation information may trigger the assessment unit 300 to assess whether the patient may be developing a manic episode. If such a conjecture is confirmed, the intervention manager 800 can timely offer interventions to the user.

Such long-term monitoring allows clinicians to obtain an accurate history of the patients' disease when there is a transition of care among clinicians over time. For example, bipolar disorder is a life-time condition, and the onset may occur during the teenage years or young adulthood. Changes in clinician is expected over the lifespan of a patient as the patient transitions from living with one's parents, going to a college to obtain further education, finding a new job in a new town, forming a new family, retiring into a retirement community, and transitioning to a nursing home or hospice care. Further, a therapy or intervention that used to work in his/her teen years or 20s might stop working in his/her later life, and a new intervention may have to be explored intermittently throughout the lifetime. The accumulation of knowledge regarding the patient's disease over years will prove to be invaluable to a new clinician who takes over the care of the patient.

This device can further provide timely intervention for patients in between visits with his/her psychiatrist and therapist. The intervention may include light therapy, music therapy, art therapy, virtual counseling, virtual meditation, biofeedback training, neurofeedback training, and positive messages, based on the patient's disease subtype. The device can even determine the most effective therapy based on a biofeedback obtained based on biometric data and clinical progression of the disease after receiving an intervention. The device can also timely remind the patient of the onset of a new manic episode so that the patient can be alerted against making rash decisions, make appropriate therapeutic adjustments such as taking an additional sleep aid, getting more exercise, or making an appointment with the healthcare provider. The device can also track a patient's physical location and alert family members or caretakers in response to the patent takes on an extended travel due to a psychotic episode or due to the increased activity resulting from a manic episode. The device can remind the patient to take a medication or even automatically administer an appropriate emergency medication. The device can provide the patient with therapeutic interventions such as neuromodulation, music or graphic therapy, mindfulness exercise, or virtual counseling therapy with an avatar. These interventions and treatments can be provided without the conscious daily involvement of a physician.

### Examples of Devices for Dynamically Determining the Subtype of a Psychiatric Disorder and Providing Personalized Medical Care Based on the Dynamically Determined Subtype

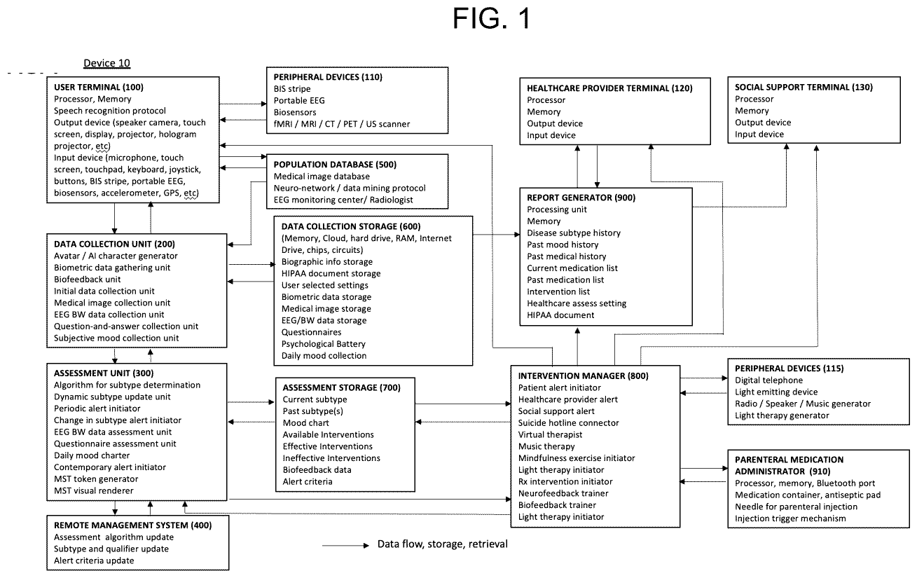

FIG. 1 depicts an example of a device 10 for dynamically determining the subtype of a psychiatric disorder and providing personalized medical care based on the dynamically determined subtype.

The device 10 for dynamically determining the subtype and providing personalized medical care may include at least one of a user terminal 100, a data collection unit 200, an assessment unit 300, a data collection storage 600, an assessment storage 700, an intervention manager 800, a report generator 900, a parenteral medication administerer 910, a healthcare provider terminal 120, a social support terminal 130, peripheral devices 110, 115, and any combination thereof. The user terminal 100, the data collection unit 200, the assessment unit 300, the intervention manager 800, and the report generator 900 may include one or more processors and memory, and some of these components may share the same CPU, microprocessor, circuit, and the like as their processing unit. The user terminal 100, healthcare provider terminal 120, social support terminal 130 may be implemented as one electronic device or separate electronic devices. The population database 500, data collection storage 600, assessment storage 700, and remote management system 400 may include memory. The memory may be a hard drive, a circuitry within an electronic device, RAM, Cloud, internet drive, chips, and the like, based on the hardware by which these components are implemented.

### User Terminal 100

The user terminal 100 of the device 10 may include a graphical user interface that is provided on a touch screen of an electronic device, or a non-touch screen visual display or a projector in combination with a keypad, keyboard, joystick, remote control, or other input device paired to an electronic device with a processing unit via a wired or wireless communication. A wireless communication may include a Bluetooth interface via a Bluetooth port or a WIFI interface via a WIFI port. The graphical user interface may be provided on a computer terminal with a display and a keyboard; a portable electronic device such as an iPad or a handheld device; a portable electronic device with a touch screen, microphone and speaker; a smart phone with a touch screen, processing unit, Bluetooth port, WIFI port, camera, microphone and speaker; a smart watch with a biosensor, microphone, speaker, and touch screen; a projector connected to a portable electronic device; a wearable device such as a wrist band or a head band or a google glasses; a wearable electronic patch with Bluetooth capacity and the like, and each and any combination thereof can serve as a user terminal 100. The user terminal 100 may include a graphical user interface that projects an image of an avatar or an artificial intelligence (AI) character that interacts with the user by asking questions or carrying on a conversation, through which information may be extracted from the user as a user input. The user terminal 100 may include a camera that allows the user to project his or her face to an AI character, allowing the processing unit to interpret the facial expression so that the AI character can respond appropriately and empathetically to the user's facial expression. The avatar may be configured to provide a facial expression back to the patient or offer words of comfort when the user is perceived to be sad through AI recognition of facial expression, voice, or word content, and may even offer to connect the patient to a suicide hotline, prayer line of different religious group, online human therapist or psychiatrist, or to alert caretakers, family members, psychiatrist, physician, social worker and the like to get emergency help as necessary. In another embodiment, the user terminal 100 may involve an audio interface that may be provided via any electronic device with a speaker and a microphone, such as Alexa, whereby an artificial intelligence character speaks and asks questions to the patient, and the microphone receives answers from the patient as an audio file which is converted into text by an automatic speech recognition technology, or other AI speech recognition protocol run on a processing unit. The speech input can be simply a “yes” or “no”, or a number, or a complex sentence that is interpreted by an AI speech recognition technology for content recognition. The speech input device can use an AI translator device to interpret different languages as selected by the user.

An input device or an output device can be paired to the user terminal as a peripheral device (110, 115). A peripheral device (110, 115) may include a BIS strip, wearable EEG electrodes, wearable EKG electrodes, medical imaging scanner, biosensors, electronic stethoscope, camera, digital telephone, biosensor patch, biosensor headband, biosensor wristband, GPS, location tracker, accelerometer, neuromuscular electric signal sensors, speaker, projector, hologram projector, smart TV, display device, light lamp for light therapy, message machine, AI character recognition device, AI speech recognition device, and the like.

According to one embodiment, the patient may be able to download an application to his or her smart phone and/or personal computer to establish the smart phone or personal computer as a user terminal 100. The smart phone may have a touch screen, which allows the user to enter a user login, initial biographical and demographic data, past medical and psychiatric diagnoses, contact information of healthcare provider, contact information of social support, medical record release form for the healthcare provider or social support based on HIPAA or other health information confidentiality law.

According to another example, the patient may be able to connect to the application via a website provided on the internet. A parallel interface may be provided on a personal computer via the internet or via a smart phone application. Data received from the patient can be stored in a data collection storage 600. The data collection storage 600 may be located in the Cloud, in the hardware of a smart phone or personal computer, on an internet drive, or a separate memory device such as an external hard drive or a designated electronic device with a memory unit. The data collection storage 600 may comply with the security requirements of HIPAA or other health information confidentiality law.

The user terminal 100 may include a processing unit comprised of circuits, semiconductor chips, a memory unit for storing and retrieving data, a touch screen, a port for attaching peripheral devices, a power storage such as a battery, a power port, a blue-tooth port to wirelessly connect with peripheral devices, and/or a WIFI port to connect with the internet or the Cloud.

A user terminal (100, 120, 130) may be also provided to a healthcare professional or a social support of the patient. The user terminal for healthcare professional interface may allow the healthcare professional to select an appropriate algorithm for treatment or allow the healthcare professional to use a default setting provided by the device manufacturer or device managing organization.

The user terminal 100 may have the ability to connect to several peripheral devices (110, 115) either through a physical port or through a Bluetooth port, to obtain biometric information via biometric data gathering unit, which is a part of the data collection unit 200.

The use of audio interface and AI character provides an emotional comfort to the patient and makes it easier for the patient to use the device. It may be easier for patients who have difficulty with concentration due to their manic episode, irritability, or depression to speak with an audio AI character or a visual audio avatar, then to read through a questionnaire on a computer screen.

Given that the emotional state of the patient is of foremost concern, the user may be provided to pick its own AI character or even be able to create his or her personal AI character by providing a picture or a video of their favorite person, pet, animal, or animation character. The AI character or avatar may be generated by an avatar generator or AI character generator of a data collection unit 200. The data collection unit may connect to a data collection storage 600 to store collected information in a memory.

Data collection Unit 200

The device 10 for dynamically determining the subtype and providing personalized medicine may include a data collection unit 200 that collects various information. The data collection unit 200 may include an avatar or AI character generator, biometric data gathering unit, biofeedback unit, initial data collection unit, medical image collection unit, EEG brain wave data collection unit, question-and-answer collection unit, and subjective mood collection unit.

A biometric data gathering unit of the data collection unit 200 may obtain biometric data from a variety of sources. For example, the biometric data may be obtained from a sensor inside a smart watch worn by the user; a BIS spectra strip or electrodes that are mounted on the user's forehead; an accelerometer inside a smart phone or a portable electronic device carried by the user; a microphone inside a smart watch or a smart phone; and/or a peripheral device (110, 115) that connects to the user terminal 100. For example, peripheral device (110, 115) may include a neurofeedback machine or a medical imaging scanner that can be coupled to the user terminal 100 via wired or wireless communication.

The biometric data may include patient's vitals, heart rate, temperature, blood pressure, movement and acceleration, physical location, glucose level, voice, facial expression, blood fat composition, perspiration, and the like. The data collection unit 200 may collect brain wave information from a BIS strip attached to the forehead of a user, EEG data collected through EEG electrodes applied to the scalp, or an fMRI information transmitted from an fMRI scanner or input by the user or a healthcare provider. The data collection unit 200 may interact with a population database that stores a large quantity of medical imaging data or EEG recording data to process the medical image or EEG recording of the user. For example, a population database 500 may provide a protocol for locating any abnormality that may be present in an fMRI or an EEG recording of the user. The protocol may be determined by performing a data mining or neuro-network processing on a large number of images or EEG recordings from a population study.

The peripheral device (110, 115) may include an electrode or a patch to be applied to the skin of the patient; a temporary tattoo with circuitry to measure biometric data while applied on the skin of the patient; a smart bracelet for continuous monitoring of heart rate, blood pressure; patches for EKG; a headband with electrodes, a helmet with electrodes, or the like to obtain for brain wave monitoring and to obtain data for polysomnography, microphone for detecting heart sound, lung sound, voice. The peripheral device (110, 115) may include a portable or wearable medication dispensing unit 910, a computer terminal 100 for answering questionnaire, imaging devices, a camera for detecting patient's facial expression, a sensor for detecting patient's heart rate, sleep pattern, temperature, a sensor for determining the amount of light in the environment, a accelerometer for determining patient's movement, including detecting akathisia, tremor or exercise, a microphone for sensing patient's voice, a speaker for producing sound, an fMRI or MRI scanner with wired or wireless data transmission, an ultrasound scanner, PET scanner, CT scanner, a digital thermometer, a blood sample analyzer, and the like.

### Initial Data Collection Unit

An initial data collection unit of the data collection unit 200 may perform a part of the initial data gathering from a user or healthcare professional. The initial data collection unit may be configured to ask a set of questions and obtain demographic information and past psychiatric and medication history about the patient. For example, the initial data collection unit may collect from the user, the user's preferred name, nickname, or a user login; date of birth or an approximate age; prior diagnosis of psychiatric disorder such as mood disorder, depression, bipolar I disorder, bipolar II disorder, panic disorder, PTSD, seasonal depression, psychotic disorder, schizophrenia, schizoaffective disorder, schizophreniform disorder, anxiety disorder, social anxiety disorder and the like.

The initial data collection unit may collect from the user, contact information for the user's social support, physicians, caretakers, family members, social worker, and/or medical power of attorney. The initial data collection unit may also explain the HIPPAA and privacy law and obtain from the user a release of information form for the social support and the physicians. The initial data collection unit may inform the user regarding available community resources such as the local suicide hotline, a prayer line, a walk-in mental health clinic, a point of contact for governmental mental health organizations, and allow the user to give permission to release information as necessary under HIPPAA or other patient privacy law.

The initial data collection unit may also collect from the user, information regarding patient's level of anxiety, depression, mania via a simple questionnaire, such as a Hamiltonian Anxiety Scale, PHQ9, mood disorder questionnaire, MMSE, and psychosis screening questionnaire. Again, the data collection can be performed by an AI character, an avatar or a speech recognition system, so that the patient does not have to read through long questionnaires on a computer screen.

The initial data collection unit may also allow the user to specify how the user would like to use the device, including whether the user gives permission to his healthcare provider or caretaker to have access to the collected data or to receive an alert in the event that the patient is determined to have a manic episode, depressive episode, prolonged sleep deprivation, suicidal ideation, onset of new disease pattern or rapid cycling of mania or depression, or other symptoms concerning for worsening of the disease. The user selection can be stored as a user selected setting in the data collection storage 600.

The initial data collection unit may collect questionnaire or data from the user via pushing a button displayed on a touch screen; voice recognition of an audio stream received via a microphone in interaction with an AI character generated by an AI character generator or an audiovisual avatar generated by an avatar character generator; a holographic avatar that interacts with the user via a holographic projector interacting with an electronic device. The initial data collection unit may include a processing unit, a microphone, a speaker, a camera, a projector, and the like. It may be equipped with an AI speech recognition technology, which may be used to interpret what the user is saying. Interactions with a favorite AI character can make it substantially easier for patients with concentration problems to answer the entire questionnaire. The data collected by the data collection unit 200 can be stored in the data collection storage 600.

### Medical Image Data Collection Unit

The medical image data collection unit of the data collection unit 200 may include a memory storage, WIFI port, Bluetooth port, and/or hardwire port, and may receive information from an fMRI scanner, MRI scanner, Ultrasound scanner, CT scanner, image database, AI or machine learning device, and/or a radiologist who reads and generates a radiologist report of a medical image.

According to one embodiment, the medical image data collection unit 200 collects or receives information regarding patient's brain, including fMRI, MRI, ultrasound, CT scan, and analyzes these data for abnormalities. For example, fMRI and MRI may be used to detect the hypoactivity or overactivity of amygdala and other brain structures, or atrophy or reduced volume of regions of brain.

According to one embodiment, the medical image data collection unit 200 may interact with a remote database of images, a population database 500, an algorithm for image processing generated in advance by data-mining tools, AI-based imaging processor that recognize abnormalities, text recognition-based radiology report processor, an image processing center that processes medical images, or even a human radiologist expert.

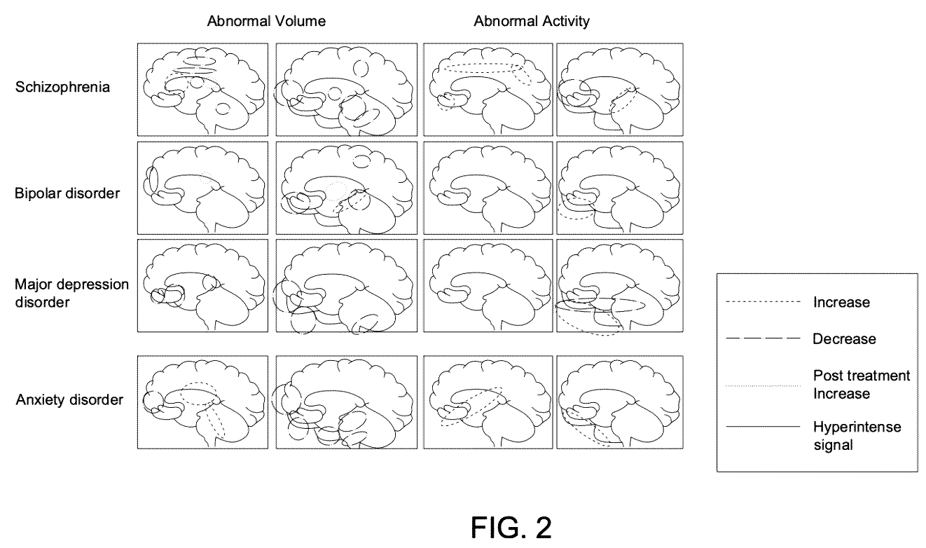

FIG. 2 depicts a chart showing examples of brain abnormalities found in different subtypes of psychotic disorders.

| Disorder | Brain Abnormalities |
|---|---|
| Schizophrenia | Overall decreased connectivity in entire cortex |
| | Decreased ReHo in frontal lobe |
| | Increased parahippocampal activity |
| Bipolar Disorder | Decreased ReHo in frontal lobe |
| | Decreased activation of cerebellum |
| | Increased activation of striatum |
| | Altered functional activity of medial |
| | frontal cortex and anterior cingulate gyri |

According to one embodiment, the medical image data collection unit may process the brain image based on an algorithm. The algorithm can be generated based on the state of knowledge regarding the relationship between psychiatric disease and structural and functional changes on brain as discovered by recent clinical studies. Some of the brain abnormalities commonly associated with various subtypes of psychiatric disorders which are currently recognized are depicted in FIG. 2B. The list of the brain abnormalities will be expanded over time as we learn more about the human brain. The algorithm can be also supplemented by using data mining or AI-based image recognition to speed up the process of recognizing brain abnormalities associated with various psychiatric disorders. Further, the processing unit of the medical data collection unit may search a radiologist's report for specific key words, such as “abnormally large amygdala” or “small . . . hippocampus”. In another example, the image can be analyzed at by a human radiologist expert to generate a report for the device 10.

### Electroencephalographic (EEG) Data or Brain Wave Collection Unit

The Electroencephalographic (EEG) data or brain wave collection unit 200 may include a memory storage, a WIFI port, a Bluetooth port, a hardware port, a set of electrodes to be mounted on the forehead or over the entire head of the patient, a polysomnography study unit that determine the onset of various sleep stages, a processing unit, electronic circuits, an AI or machine learning device for interpreting the EEG Data Collection, and may even connect to a medical facility or lab staffed with a human epileptologist, neurologist, EEG readers, or EEG technicians or to an EEG monitoring center.

The EEG Data Collection Unit may include a wearable EEG data collection device, a headband with electrodes, an electrode helmet, or a skin patch with electrodes which the user may apply on their forehead. The user may wear the EEG data collection unit as they go to sleep obtain a polysomnogram or wear the device while awake and/or asleep for continuous monitoring over hours, days or longer. The wearable EEG collection device may interact with a portable electronic device such as the user's phone or personal computer via Bluetooth or WIFI port.

According to one embodiment, the EEG Data Collection Unit comprises a limited number of electrodes placed on the forehead of the patient, as a strip or a patch. The EEG Data Collection Unit may be a BIS strip, a 10-20 system EEG helmet, or a commercially available full 10-20 system EEG wearable device.

The EEG data collection unit 200 receives information regarding patient's brain activity via the electrodes and transmit the collected data to an electronic device via Bluetooth or WIFI or other wireless terminals, or even a wired connection. The electronic device that receives the information may be equipped with an AI recognition device for detecting different stages of sleep. For example, the device may be able to recognize stage of sleep such as wake, N1, N2, N3 and REM sleep based on the signals received from electrodes placed on the forehead and determine the sleep architecture of the patient each night. Further, the AL recognition device may be able to detect whether the architecture is abnormal, such as the reduced REM latency observed in some depressed patients and bipolar patients, and reduced quantity of sleep observed during manic episodes in bipolar patients. The EEG data collection unit 200 may be also able to detect whether patient is relaxed or agitated, based on patient's eye blinking rate, brain wave collected through EEG or a limited number of electrodes, heart rate determined by an optic sensor, and the like.

The EEG Data Collection Unit may be configured to transmit important findings, such as a deviation in sleep architecture, abnormalities in the sleep architecture, patient's level of mood or agitation, eye blinking rates, and the like to the Assessment unit.

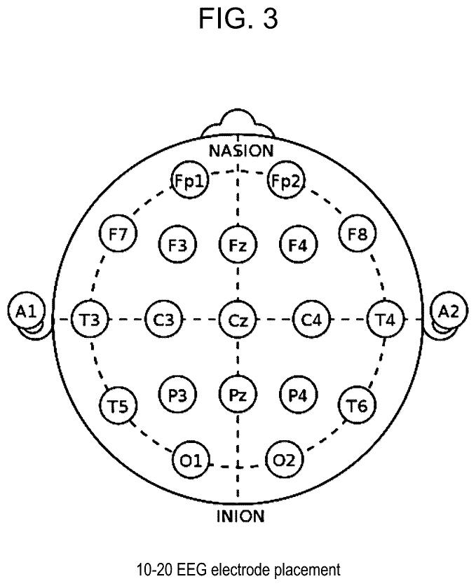

FIG. 3 illustrates an example of a 10-20 electrode placement for an EEG recording. The electrodes were traditionally glued on the scalp of the patient's head. However, the electrode gluing session can take about 2-3 hours. A BIS strip is a sensor including a limited number of electrodes that can be applied to the patient's forehead. The electrodes obtain information about the patient's electrical brain activity. The information is processed and translates into a number between 0 (no cortical brain activity measured) to 100 (normal awake and alert patient).

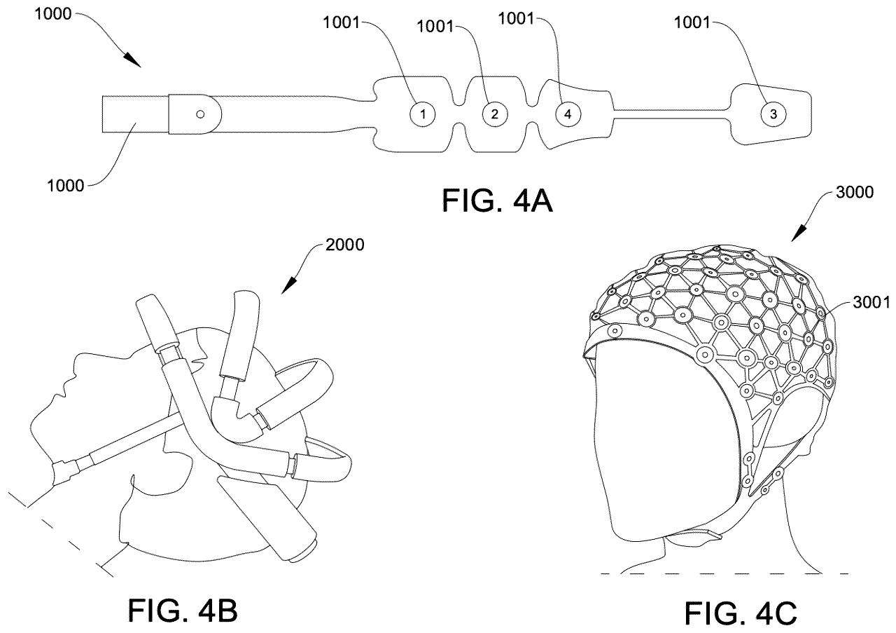

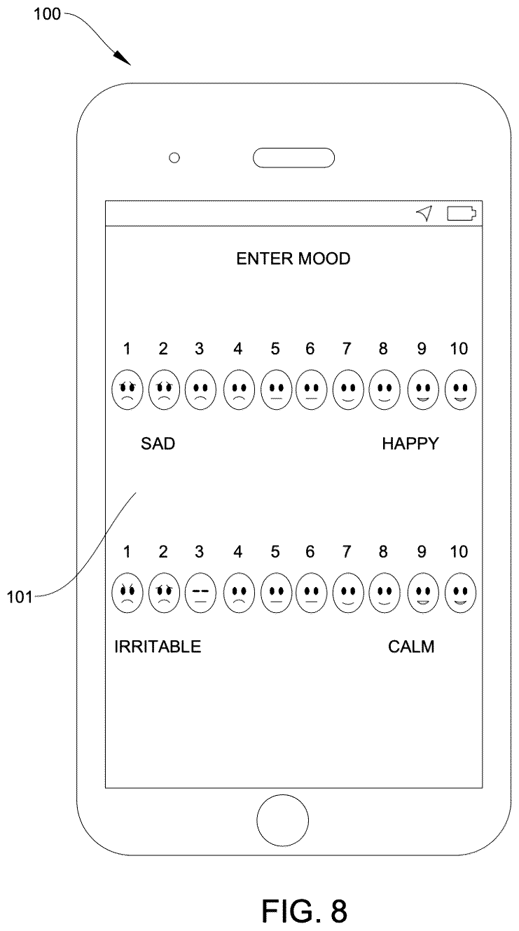

FIG. 4A depicts an example of a BIS strip. FIG. 4B depicts examples of portable EEG helmets. A BIS strip can have a small number of electrodes. For example, two-electrode, four-electrode, and six-electrode models are commonly used. An example of a commercially available BIS strip from Medtronic is depicted in FIG. 8A. The BIS strip can be easily applied to the forehead of a patient. The Medtronic BIS strip establishes a wired connection to a BIS VISTA monitor. However, this disclosure is not limited to these examples. A different number of electrodes can be arranged in a desirable manner and mounted on the forehead. According to another example, six electrodes can be arranged on a patch to be applied to the forehead. A WIFI electronic device in a shape of a headband can be provided with six electrodes to transmit the signal via Bluetooth to a user terminal. Alternatively, a wired connection to the electrodes can be achieved to a portable device such as a smartphone which is carried by the patient. Further, FIG. 8B depicts Zeto and eego EEG system, which are currently commercially available EEG monitoring headgear. A number of portable 10-20 EEG head gears are also now commercially available, and some devices have received FDA approval.

It has been determined that BIS strip can provide some information regarding the sleep stages. For example, light sleep occurs at BIS values of 75-90, slow-wave sleep occurred at BIS values of 20-70, and rapid eye movement sleep occurred at BIS values of 75-92. Thus, the BIS strip can be used to monitor sleep architecture.

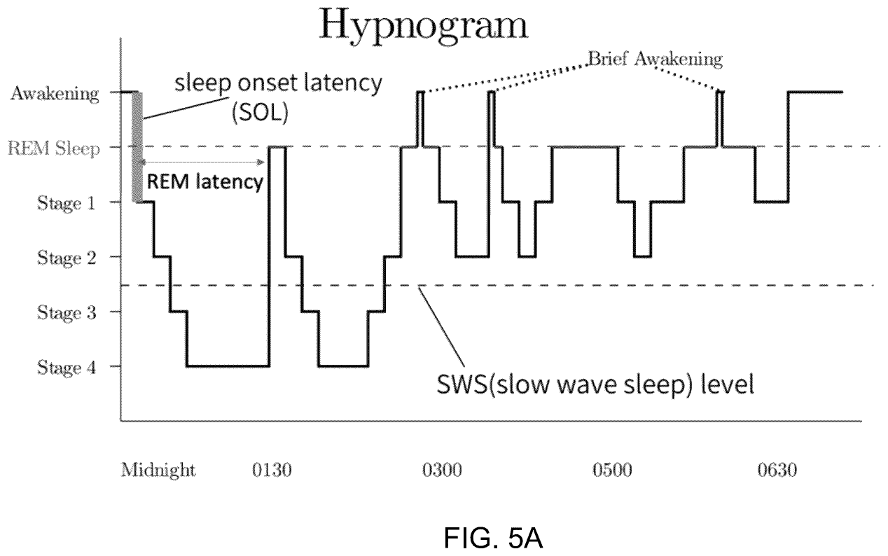

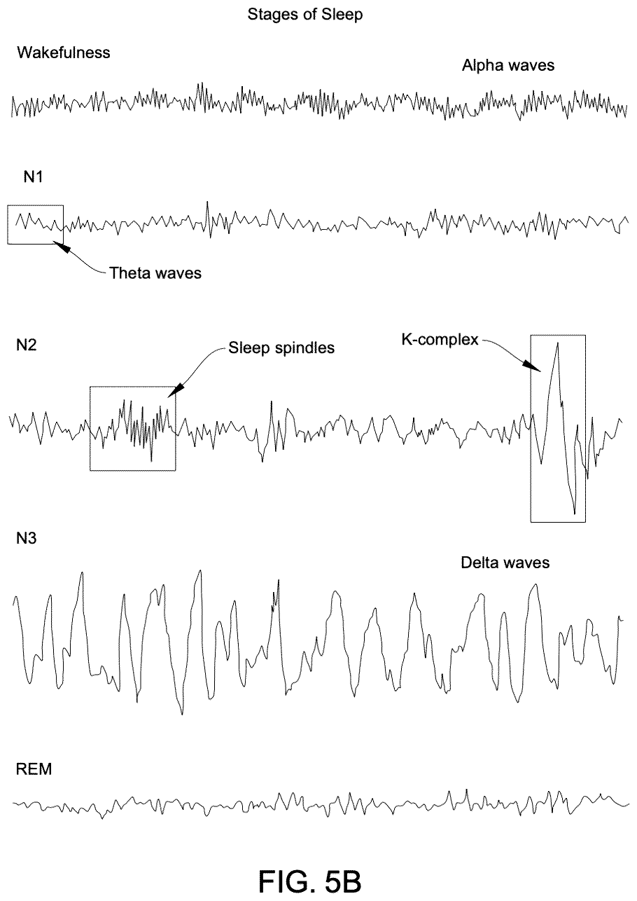

FIG. 5A depicts an example of a normal sleep architecture. A reduction in REM latency has been noted to be associated with major depression. Thus, a subtype qualifier can be “with reduced REM latency,” for example. Further, onset of manic episode is often marked by decreased need for sleep and increased activities, which can be detected by studying the sleep pattern.

FIG. 5B depicts EEG signals during various sleep stages.

Neurofeedback is a type of biofeedback that may involve taking a full 10-20 EEG recording or 10-10 EEG recording. The technique may involve helping the patient learn to achieve relaxation, concentration, focus and the like. The user can be presented with real-time feedback based on the electrical activity of the brain measured by the EEG in order to reinforce healthy brain function. Behavioral conditioning such as a reward system may be incorporated to the training.

Traditionally, the electrical activity from the brain is collected via EEG electrodes placed on the scalp of the patient, with feedback presented using a video display or sound while the patient is listening to certain sound or watching a video on a display or engaging in a computer game.

Given that wearable devices make it possible for patients to engage in neurofeedback sessions at the convenience of their home, patients can elect to expose themselves to a variety of their daily activities such as reading, meditation, listening to a religious scripture, and the like, while the brain electrical activity is being analyzed on a processor in a continuous real-time fashion.

According to one embodiment, the user terminal 100 can allow a user to select an activity such as reading a religious scripture while a neurofeedback session is performed. The intervention manager 800 can display the bible verses or read aloud the bible verses by an AI character, while a peripheral device comprising of EEG electrodes is mounted on the user's head or forehead to allow a real-time analysis of the brain activity by a processor of a biofeedback unit. The analyzed data can be provided to the user as a biofeedback. Thus, it is possible to determine which daily activities performed by the user causes calming effect. The user no longer has to travel to a remote neurofeedback center or get specialized equipment to engage in a neurofeedback session.

Further, one of the limitations of current neurofeedback training is that everyone has different brain wave patterns because everyone's brain has slightly different connectivity and structure. According to one embodiment, the user can wear the electrodes continuously over several minutes to days, overnight, during daytime, and the like. While wearing the electrodes, the user can enter his/her current mood via the data collection unit. Thus, the assessment unit or processor of the device will be provided with an opportunity to learn about the user's baseline EEG signal in various settings, such as the user's wake state, different stages of sleep, irritable state, calm state, euthymic state, depressive state, manic state, psychotic episodes, and the like. The learned pattern can be used to determine the target neurofeedback EEG signals specific to the patient.

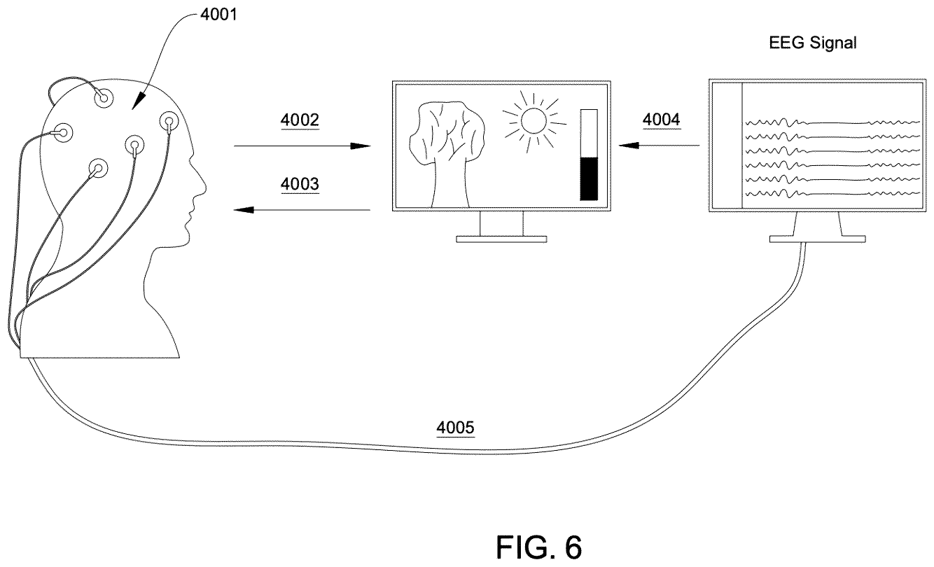

FIG. 6 depicts a schematic diagram explaining an example of a method of providing neurofeedback training.

The patient 1001 is provided with an audio and/or visual input through a monitor, while the patient's brain electric activity is measured via EEG electrodes placed on the patient 1001. The brain activity is analyzed real-time.

Since various wearable EEG monitoring units are available, the neurofeedback processing can take place for hours without the patient's conscious involvement. Further, the device 10 can allow the patient to interact with his/her natural surroundings.

### Question and Answer Collection Unit

The question-and-answer collection unit of the data collection unit 200 may collect simple information such as the patient's subjective mood for the day, a portion of the day (e.g. AM and PM), or collect answers to more complex issues such as patient's past medical history, past psychiatric history, family history of illness, history of psychological trauma, physical injury to brain, history of sexual abuse, developmental history, cognitive or behavioral disability, patient's feeling towards his/her parents and siblings, history of neglect or abuse as a child, concern for safety, and/or endocrine or autoimmune abnormalities. This crucial information can be made available to healthcare professionals by being transmitted to the assessment unit 300 and stored in a database or memory storage 600 where it can be retrieved by the healthcare professionals.

The question-and-answer collection unit may also collect from the user, information regarding patient's level of anxiety, depression, and/or mania via a questionnaire, such as a Hamiltonian Anxiety Scale, Mood Disorder Questionnaire, Mini-Mental State Examination (MMSE), Montreal Cognitive Assessment, and/or Psychosis Screening Questionnaire. The question-and-answer collection unit may ask series of questions targeted to clarify the correct diagnosis among candidate diagnoses being considered for the user. For example, the DSM-5 diagnosis criteria of candidate diagnoses can be used to generate appropriate questions to clarify the correct diagnosis for a patient, such as “Have you had a time period of sevens days or more in which you felt you did not need as much sleep, and you were speaking really fast?” (Bipolar I Disorder). The question-and-answer collection unit may obtain information regarding patient's cognitive abilities by allowing the user to play a cognitive video game on a portable electronic device, such as the user's smartphone. The question-and-answer collection unit may object information specific for different modalities of talk therapy. For example, questions directed to determine the superego, ego, and id structure of the patient can be asked, such as “Do you think you become very critical of your actions or mistakes?” An entire battery of psychological test can be performed via the question-and-answer collection unit.

The data collection can be carried out in the format of a list of questions displayed on a touch screen, and the user can manually select an answer via the touch screen. Alternatively, an AI character or avatar coupled with speech recognition device can be provided. The AI character may provide audio through a speaker of a smart phone, may be displayed on a touch screen, and/or both. The user may be able to carry out a conversation with the AI character, allowing the device 10 to collect the necessary information to establish the diagnosis. An interaction with an AI character makes it more comfortable for patients to complete a long questionnaire, especially if the patient has difficulty concentrating due to an active depressive episode, psychosis, or an active manic episode. The question-and-answer collection unit may be equipped with algorithms to score various questionnaires or battery of psychological tests to determine the most likely subtype of a patient's psychiatric disorder.

The question-and-answer collection unit can also utilize a camera provided on the user terminal to capture the patient's face or ask the patient to show certain parts of the body for a digitalized cursory physical exam. For instance, if there is a concern for extra-pyramidal symptoms, tremor, dystonia, eye blinking or lip smacking, the AI character can ask the patient to show his/her face and use the camera to analyze for any eye blinking or lip smacking. The AI character can ask the patient to also stick out tongue or show the shoulder or arm, and determine whether there is any tremor. The AI character can also ask the patient to hold out their arms while holding the phone, and use the accelerometer of the phone to detect any tremor. If there may be a tremor, this information can be used to alert the physician, for example.

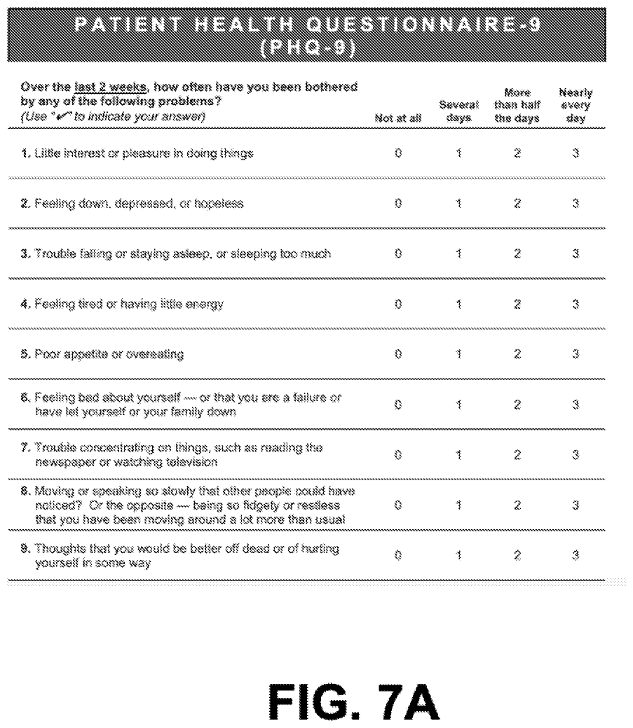

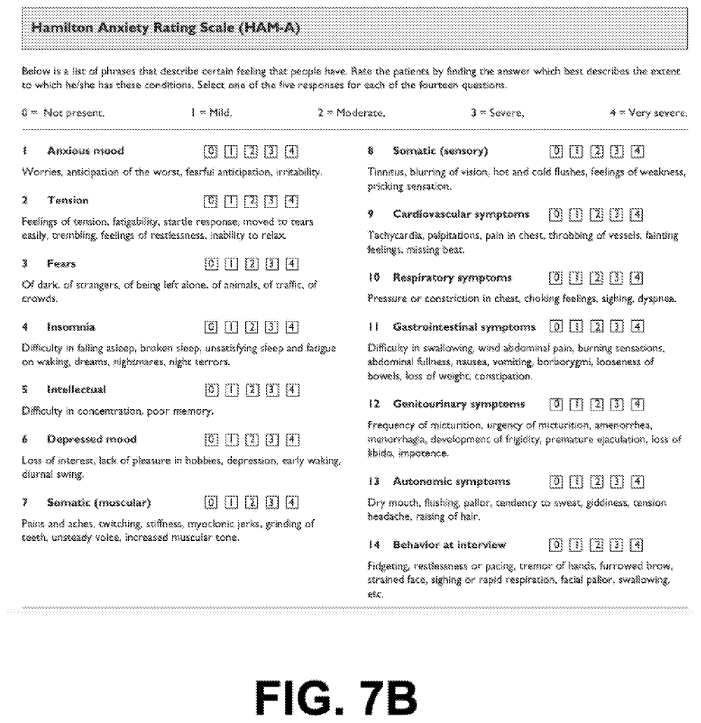

FIGS. 7A and 7B depict examples of questionnaires that can be used to clarify the subtype of a patient. Various questionnaires, sets of questions, and battery of psychological tests may be used to clarify the subtype of a patient. For example, the device 10 may employ patient health questionnaire-9 (PHQ-9), Hamiltonian anxiety scale, personality inventory for DSM-5 (PID-5), Mood Disorder Questionnaire, Bebbington psychosis screening questionnaire, clinician-rated dimensions of psychosis symptom severity, neuropsychological test and assessment, and the like.

### Genetic and Endogenetic Data Collection Unit

A genetic and endogenetic data collection unit of the data collection unit 200 may receive information regarding patient's genetic information, family history, or endogenetic information. Oxidative stress and neuroinflammatory markers are implicated in many psychiatric conditions and are considered to be a part of endogenetic. Schizophrenia and bipolar disorder are shown to exhibit substantial heritability. The genetic information can be stored in the data collection storage 600, and assessed by the assessment unit 300 to determine whether the subtype of the patient needs to be updated. If it is determined that the patient has several schizophrenia related genes, his father also had schizophrenia, and that his father's disease was responsive to Invega, there is a high likelihood that the patient may also respond well to Invega. By combining the family psychiatric history with the genetic information, further information can be extracted regarding the efficacious treatments for a patient.

### Biofeedback Unit

Biofeedback is a process that enables an individual to learn how to change one's physiological activities for the purpose of improving mental and physical health. Wearable devices, biosensors, camera, portable device and/or peripheral devices 110, 115 can be used to monitor physiological activities of the body such as the electrical activities of brain, heart function, breathing, muscle activity and skin temperature of the user. The received information are processed by the biofeedback unit of the data collection unit 200 to inform the user regarding the user's emotional state and physiologic state. When an EEG is collected to inform the user regarding brain function, the biofeedback mechanism is called neurofeedback. The information can aid the user in determining which intervention or activity helps to calm down the user, increase concentration, or the like. The device 10 can also use the biofeedback information to determine which intervention is efficacious and store the list of efficacious interventions for the individual user in the assessment storage 700. Thus, when the user is suffering from a similar symptom or state of mind, the assessment storage 700 can be accessed to determine which intervention may be offered to the user.

For example, if a music therapy is tried on a patient when the patient is depressed, the assessment unit 300 can assess whether the music therapy has been efficacious. Next time the patient is depressed, an AI avatar displayed on the user terminal 100 can ask the user whether the user wants to participate in a music therapy to feel better. If the user tried a neurofeedback therapy and it was determined to be efficacious in calming down the user during a manic episode, the device 10 can offer a neurofeedback therapy session the next time the user experiences a manic episode.

### Subjective Mood Collection Unit

According to one embodiment, the collection unit can collect the subjective mood of the patient daily (e.g. once per day), twice a day (e.g. AM and PM), or more than twice per day (three or more times). For example, the subjective mood can be solicited from the user via a graphic user interface showing emoji's and/or corresponding numerals out of 10, 1 being very unhappy, 5 being euthymic, and 10 being very happy. In an alternative embodiment, an AI character may verbally ask the user about his/her subjective mood at the moment. Further, other emotions such as irritability, grandiosity, anger, calmness, and confusion can be assessed in the same manner. Some bipolar patients experience their manic episode to be accompanied by “irritability” rather than “grandiosity” or “happiness.” Thus, for a bipolar disorder, an algorithm of the assessment unit 300 can be set based on this clinical finding to check on the “irritability” of the user as well as whether the user is euthymic, dysthymic, hypomanic or hyperthymic. The subtype for such a patient can be further defined as “with irritability during manic episode.” As such, the next time the patient has reduced sleep needs, the device 10 can be sure to check on irritability.

FIG. 8 depicts an example of graphical user interface that may be used to obtain subjective of a user. In the example depicted in FIG. 8, the emoji and numerals (1-10) are displayed on a smart phone to allow the user to select the current subjective mood. The user can enter the mood by a click on a touch screen of the smart phone.

The subjective mood collection unit of the data collection unit 200 may request the user to input the subjective mood in the morning and in the evening to determine whether a diurnal shift exists in mood. If diurnal shift exists, the patient's disease subtype can be updated to indicate that the patient has a diurnal mood fluctuation. The subtype can be further defined with a qualifier, “with diurnal mood fluctuation,” for example.

Further, the assessment unit 300 may render the daily mood data in a high-level graphic rendering by obtaining an average mood over a short time period and plotting the average mood over the short time period on a mood chart. For example, the short time period may be 2 days, which will include 4 data points if recorded in the AM and PM. The short time period may be 3 days, which will include 6 data points if recorded in the AM and PM. It may be 5 days, averaging up to 10 data points. It may be 7 days, averaging up to 14 data points. An outliner data entry can be removed, and a linear regression can be applied to obtain a smooth curve that shows mood fluctuations over time. For instance, a bipolar patient may exhibit 7-days or more of elevated mood period during a manic episode, followed by a depressive mood period that may last much longer. The mood chart makes it possibly to dynamically determine the onset of a manic episode. For example, the assessment unit 300 may trigger a contemporary alert initiator to further investigate the mood of a user if the user has a history of bipolar disorder or seasonal affective disorder and exhibits hypomanic mood, manic mood or depressive mood for a pre-determined length of time. For example, the alert can be initiated for depressed or manic mood lasting more than 3 days, 4 days, 5 days, 6 days, 7 days or more. In response to the contemporary alert initiation, the data collection unit 200 can perform further investigations as to whether the user also has other manifestations of the disorder, such as distractibility, increased energy, pressured speech, suicidal thought, sleep issues, and the like to determine whether the patient is experiencing the onset of a seasonal affective disorder, manic episode, depressive episode, and the like.

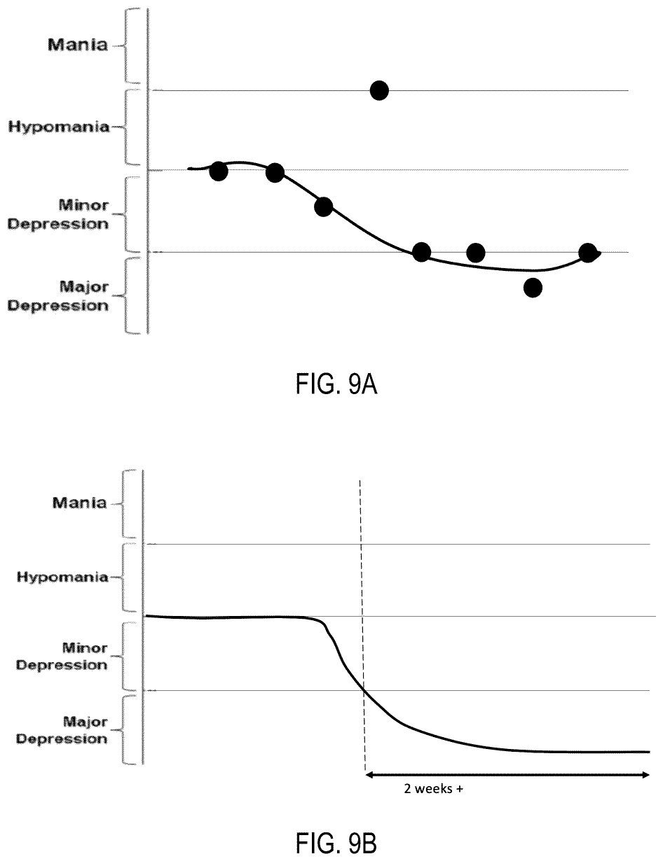

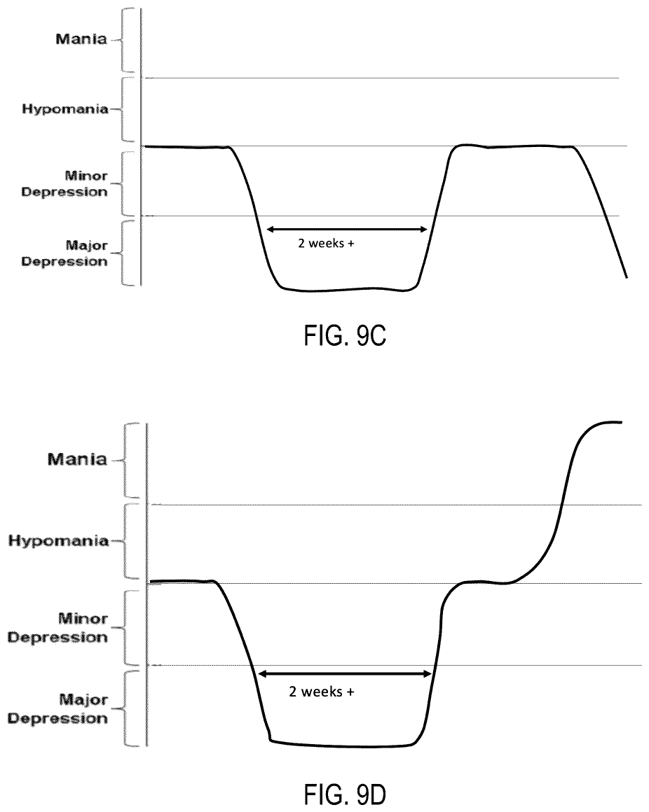

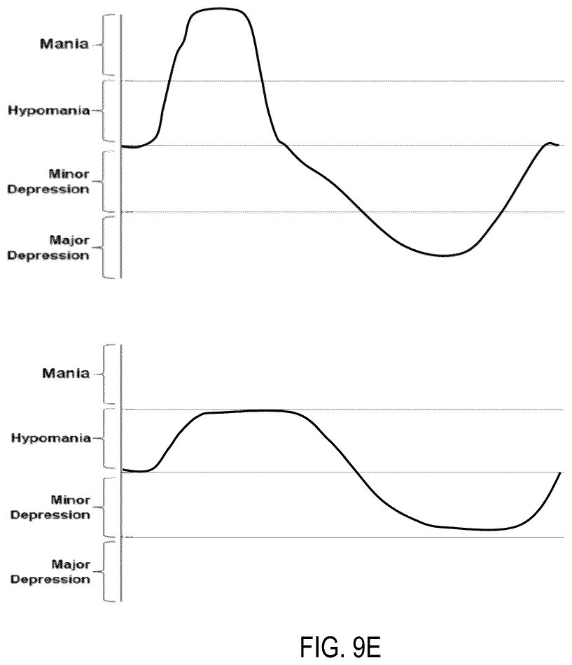

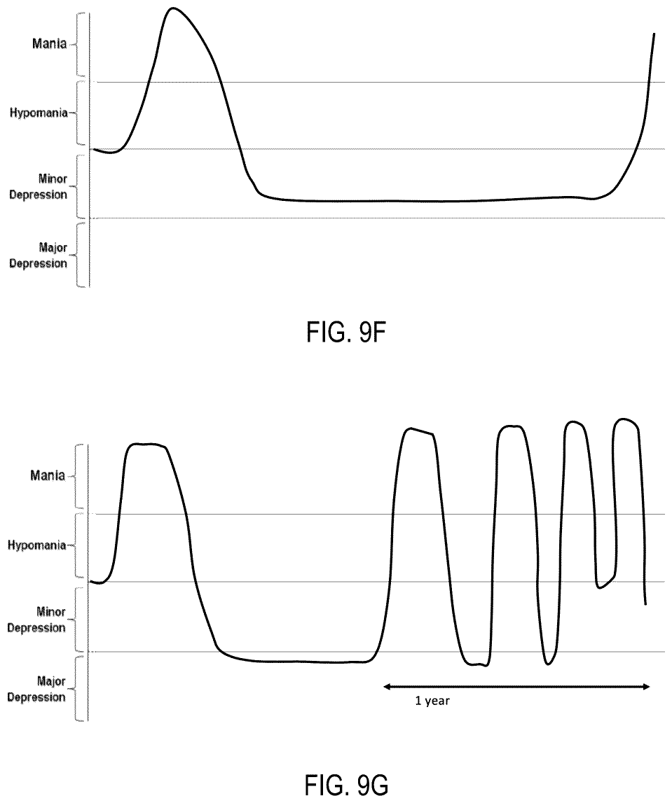

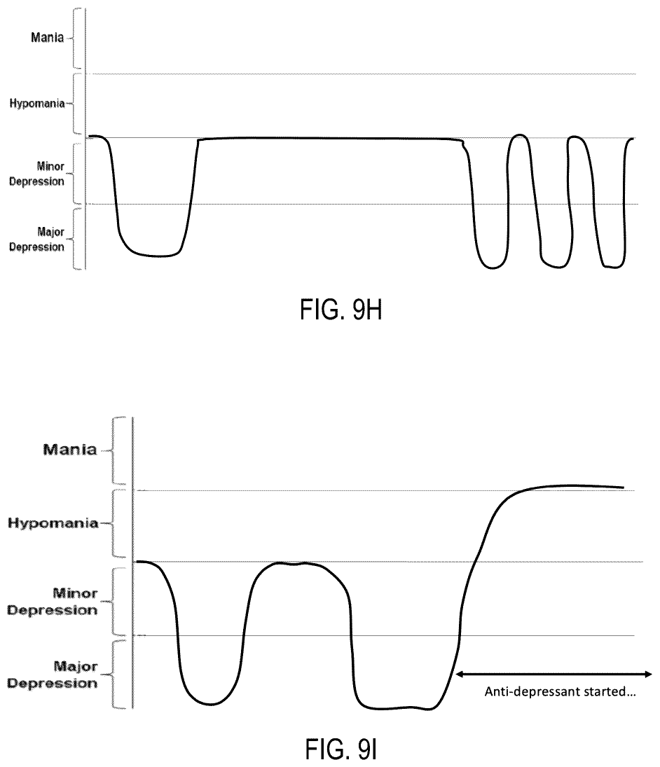

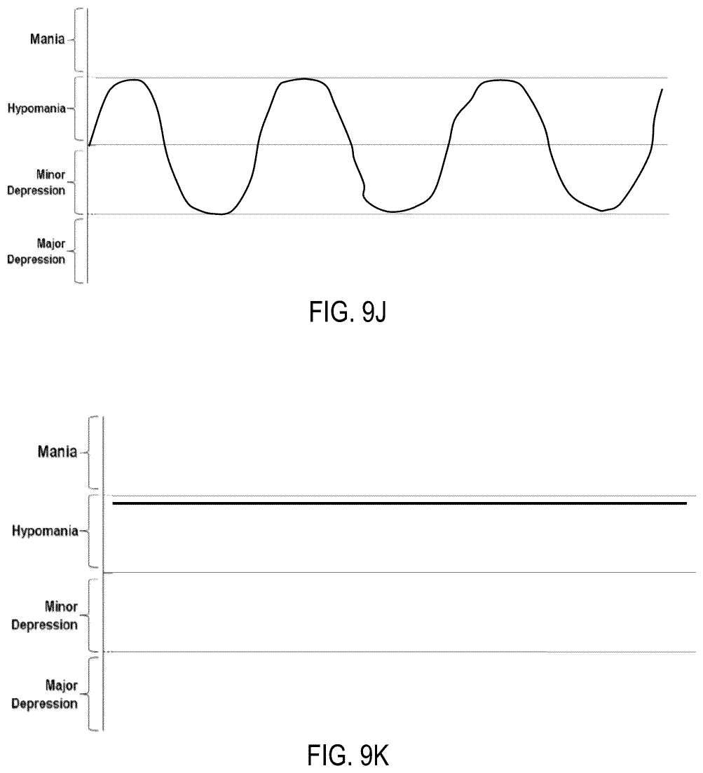

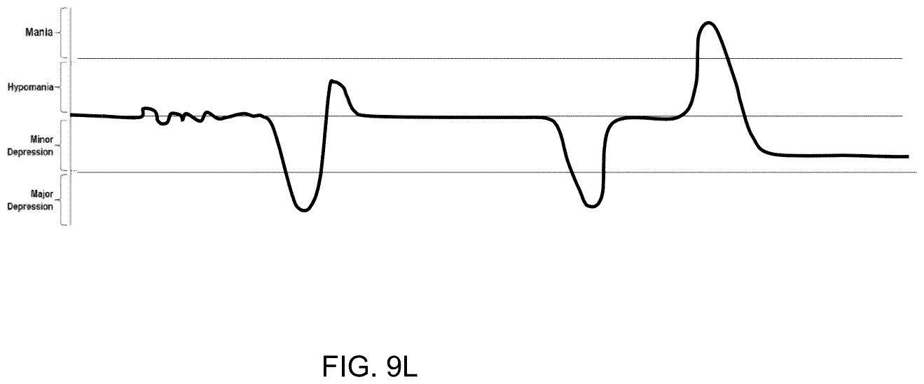

FIGS. 9A to 9K depict examples of mood charts obtained from different users, the mood charts containing data obtained over a various length of time, such as days, weeks, months, or even years.

For example, FIG. 9A illustrates how the mood rating input by the user can be plotted into a high-level graph over time. Such a high-level graphic representation can be provided to a clinician or the user to help them better manage the user's symptoms.

FIG. 9A depicts an example of a graphical rendering of subjective mood input entered over 4 days. The patient entered 5 (AM, first day), 5 (PM first day), 4 (AM second day), 7 (PM second day), 3 (AM third day), 3 (PM third day), 2 (AM fourth day), 3 (PM fourth day). The entry of 7 on the second day PM is an outlier, and eliminated from the chart. Linear regression line has been applied to determine the curve.

Monitoring the mood over an extended length of time can aid the clinician and the patient to quickly identify the onset of a mood disorder. For example, FIG. 4B depicts the onset of possible major depressive disorder. The patient has been feeling low over 2 weeks or more. Other clinical parameter necessary to establish the diagnosis of major depression includes the impact of the patient's sleep, concentration, appetite, energy level, anhedonia, loss of interest, psychomotor retardation, suicidal ideation, and the like. By dynamically checking on the mood of the patient, the current subtype of the disease can be quickly updated by the assessment unit based on newly acquired mood information obtained through the subjective mood collection unit. Further, any update of the current subtype is an important information for which an alert should be raised. Accordingly, the subtype update can initiate the contemporary alert initiator to send an alert to the intervention manager 800. The intervention manager 800 can initiate a patient alert via the patient alert initiator 800 or alert a healthcare provider via the healthcare provider alert initiator 800, or initiate a social support alert via the social support alert initiator 800. The intervention manager 800 can also gather more information regarding the patient's state of mind by activating the suicide hotline connector 800. The suicide hotline connector 800 can ask the patient whether the patient is experiencing suicidal ideation and offer to call the suicide hotline on the behalf of the user via a digital telephone 115.

Further, the charting of mood at a set time in the morning and a set time in the evening allows the device 10 to determine whether there is a diurnal pattern of depressive mood or irritable mood. The current subtype can be updated to reflect “with diurnal pattern.” Appropriate treatment can be implemented, such as light therapy at noon time, in consultation with the healthcare professional. The healthcare professional terminal 120 can provide the healthcare professional with the diurnal pattern in a report generated by the report generator 900, and allow the provider with a list of available intervention. If the healthcare provider decides to implement the light therapy, the user selected setting stored in the data collection storage 600 can be updated to implement the intervention. Daily, new discoveries are being made regarding new clinical interventions that may work for a specific psychiatric disorder. The remote management system 400 may be managed by the manufacturer of the device 10, or a group of system management professionals who update the list of available interventions and update which intervention may be offered for each subtypes of conditions based on the ongoing clinical research and discovery regarding new effective treatments.

FIG. 9B depicts an example of a graphical rendering of subjective mood of a patient over several weeks. The patient was initially euthymic but started to experience depressive mood. According to DSM-5, when a patient remains in depressive mood for 2 weeks or more, and have other symptoms of major depressive disorder outlined in DSM-5, a clinical diagnosis of major depressive disorder can be made.

FIG. 9C depicts a patient with a major depression with a new onset recurrent episode. FIG. 9C is concerning for a new onset recurrent episode of depression in a patient with major depression disorder. In response to such a mood swing, the subtype of the disease can be updated from a major depression without recurrence to a major depression “with a possible recurrent episode” or “currently in depressive mood for X number of days,” and the like. The updating of a subtype is a significant clinical event, and thus, the assessment unit 300 can activate the contemporary alert initiator. In response, further information may be collected by the data collection unit 200 to determine whether the subtype should be upgraded to “with a recurrent episode.” Further, the intervention manager 800 can be initiated to provide appropriate intervention and treatment and provide appropriate alert to the user, healthcare professional and/or social support of the user.

FIG. 9D depicts an onset of a manic episode in a patient who was believed to have a major depressive disorder. The current subtype can be dynamically updated based on the new finding, appropriate alerts can be generated, and appropriate interventions can be offered. In other words, the new information can trigger an updating of the subtype by the assessment unit 300, generate an alert by the assessment unit 300, cause the data collection unit 200 to collect more data, and/or initiate the intervention manager 800 to offer an intervention.

FIG. 9E illustrates that diagnosing a patient can be a dynamic process that requires continuous updating of the subtype. FIG. 9E compares a bipolar I disorder patient to a bipolar II disorder patient. The data collection unit 200 can obtain additional information such as PHQ9 to clarify the subtype. Whether the mood elevates to a mania level or to a hypomania level clarifies whether diagnosis should be a bipolar I disorder or a bipolar II disorder. A manic state defines bipolar I disorder, whereas a hypomanic state may be present in a bipolar II disorder patient.

Subtypes such as whether there is psychosis, delusion, hallucination should be further investigated to narrow down the subtype. Also, comorbid conditions can be explored. For example, bipolar disorders have a high comorbidity with seasonal affective disorders. Accordingly, the mood can be charted over several months to determine whether there is also a comorbid seasonal affective disorder. The mood can be charted to determine the presence of a depressive episode that starts in the Autumn, such as in November in the East Coast of the United States, for example. The length of day or the amount of light throughout the day can be also input via the camera or extracted from pictures taken by a smart phone. The length of day or the amount of light can be approximated based on the physical location obtained from a GPS in the user terminal 100. If a patient is depressed during the Winter or when the daylight is short, and improves in mood during Spring, the diagnosis of seasonal affective disorder is very likely. If this pattern repeats the next year, the diagnosis becomes more likely. If it is determined that a seasonal affective disorder is present, the subtype should be updated to “with seasonal affective disorder.” Thereafter, an appropriate alert can be initiated at the change of season each year, thereby allowing the patient to anticipate or acknowledge the onset of a depressive season. An intervention such as a light lamp therapy can be offered to the patient during the Winter, and its therapeutic efficacy can be determined by the biofeedback unit and/or based on mood input collected by the subjective mood collection unit. An alert for a potential manic episode can also be provided to the patient in early Spring so that the patient can discontinue the use of the light lamp or stop an antidepressant that the patient may have been taking during the Winter. With years of data, such intervention can be offered at a specific time of the year in advance of experiencing any manic symptoms.

FIG. 9F depicts an example of a patient with bipolar I disorder who had one episode, followed a prolonged depressive period. Bipolar I disorder is defined as the occurrence of at least one manic episode. The full criteria can be found in DSM-5.

The recent change in mood to a manic state from a depressive state is concerning for the development of a new onset of manic episode. The subtype of this patient can be updated to “bipolar I disorder with possible recurrent manic episodes.” Since there is a potential onset of a new episode of mania, appropriate contemporary alert can be initiated for further investigation, and interventions and treatment can be initiated by the intervention manager 800.

FIG. 9G. depicts an example of the onset of a rapid cycling mania in a bipolar I disorder patient. Charting the mood makes it very easy to recognize the rapid cycling. Having four or more manic episodes within one year period defines “rapid cycling.” The subtype may be updated appropriately, proper investigations can be initiated, and the clinician can be alerted to take a closer look at the therapeutic interventions offered to the patient.

FIG. 9H depicts an example of the progression of a single episode of major depressive disorder to a recurrent disease. The number of episodes can be counted to update the subtype. For example, the subtype can include the qualifier “with 4 repeated depressive episodes.”

FIG. 9I depicts an example of a development of hypomanic episode in a patient with a major depressive disorder subsequent to starting an antidepressant. An alert can be initiated for a close monitoring in the event that the hypomanic state converts to a manic state. The subtype may be updated from major depression to “bipolar III disorder”, or “with a substance-induced hypomania”. The healthcare professional can receive an alert to consider discontinuing the antidepressant and to start a mood stabilizer in the event that the patient is determined to have a bipolar disorder. The diagnosis can be further refined by continuing to monitor the patient, and by having the patient answer additional questions regarding sleep pattern, irritability, grandiosity, distractibility, energy level, insight, decision making capacity, etc.

FIG. 9J. depicts an example of the mood chart of a patient with cyclothymic disorder. The patient may be initially believed to have a hypomanic episode or a mild depressive episode. However, by monitoring the mood over an extended time, the subtype can be updated to a cyclothymic disorder.

FIG. 9K depicts an example of the mood chart of a person who has hyperthymic temperament. A person can have either hyperthymic temperament or a dysthymic temperament. These conditions are not considered pathological if they do not interfere the daily function of the person. However, a long-term monitoring may be useful due to a greater risk of developing bipolar disorder or depression.

FIG. 9L depicts an example of a lifespan of mood change in a patient. The patient experienced a depressive episode in his teenage years. However, the patient gets another recurrence in setting of a substance use as an adult in setting of life events, such as divorce. Substance use leads to a manic episode, potentially requiring a change of subtype to bipolar III disorder. Thereafter, in the late stage of life, patient displaces depressive temperament. Over the course of one's life span, several updates may have to be made to the subtype.

### Assessment Unit 300

The assessment unit 300 may include a processing unit, a memory storage, a cloud storage, a WIFI port, a Bluetooth port, a hardware port for wired communication or other wireless communication to connect with an external database or external or remote computer system.

The assessment unit 300 receives data collected by the initial data collection unit of the data collection unit 200. The assessment unit 300 may also receive data collected by an EEG or brain wave collection unit, a battery of psychological tests or a collection of questionnaires collected by the question-and-answer collection unit, genetic and endogenetic information collected by the genetic/endogenetic collection unit, biometric information collected by the biofeedback unit, a set of subjective mood information collected by the subjective mood collection unit. The biometric data may be continuously monitored while the patient is wearing the sensor. This information may be assigned appropriate points to determine the most likely subtype of the patient's psychiatric disorder.

The subtype may be partially based on symptoms, as are the DSM V categorization, as well as whether the patient has a recurrent disease, whether the patient experienced any psychosis related to mood symptoms. The subtype may be also based on the presence of phenotypes such as whether the patient has a seasonal affective disorder, the approximate month in which the seasonal affective disorder starts to manifest itself, and endophenotypes such as sleep structure instability determined by analyzing EEG pattern or brain wave during sleep, a reduction in REM latency, dysregulation of motivation and reward, executive function dysfunction, functional brain abnormalities observed by fMRI, structural brain abnormality, abnormal cognitive performance, impaired ability to recognize facial expression. The term “endophenotype” refers to an internal or intermediate phenotype, such as that which is not obvious to an unaided eye, that fills the gap between genes and distal diseases. Endophenotypes may provide a means for identifying the upstream traits underlying a clinical phenotype, and may provide the “downstream” results from genetics. The subtype may be also based on the presence of a genetic marker, specific genes that predispose a patient to develop a disorder, neuroinflammatory markers found to be elevated in certain disorder, specific EEG patterns, and family history of psychiatric disorder. Neuroinflammatory markers may refer to high concentration of pro-inflammatory cytokines in serum or CSF, elevated plasma level of IL-6, elevated serum level of IL-10, elevated serum level of tumor necrosis factor (TNF), increased serum chemokine levels, elevated C-reactive protein (CRP), and the like. These molecular endophenotype markers may be elevated in a bipolar disorder patient even while the patient is in euthymic mood. Structural brain abnormalities may include a reduction in volume of anterior cingulate cortex (bipolar disorder), an increased thickness of cortex (bipolar disorder), and the like.

Functional brain abnormalities may include increased Regional Homogeneity (ReHo) in left parahippocampal gyrus and right para hippocampal gyrus (MDD), decreased ReHo in right middle occipital gyrus (MDD), enhanced connectivity between anterior subgenual cingulate gyrus to dorsomedial frontal lobe and left dorsolateral frontal lobe (MDD), decreased connectivity between insula, amygdala and precuneus (MDD), decreased functional connectivity of amygdala to ventral lateral prefrontal lobe, insula, middle temporal/superior gyrus, cerebellum and occipital lobe, enhanced connectivity of amygdala to bilateral temporal poles (MDD), and/or reduced connectivity of amygdala to left ventral prefrontal lobe (MDD).

Answers to questionnaires regarding childhood trauma and the presence or absence of traumatic brain injury can also define a subtype, such as “with childhood psychological trauma”, “with traumatic brain injury”, “with CVA”, “with denial of childhood psychological trauma,” “without history of traumatic brain injury,” “without history of CVA” and the like. These qualifiers are meaningful because childhood traumatic experience can cause biological, molecular, or structural changes to the brain due to the neuroplasticity of the growing brain. Thus, these qualifiers provide information regarding what type of intervention may work for a specific patient. Likewise, a depression caused by a stroke to certain areas of the brain responds differently to medications as compared to a genetically inherited depression. Thus, these qualifiers can be used to assign a point for certain diagnosis and can provide information regarding the likelihood that certain treatment will work better than others. Responsiveness to pharmaceutical agents, electroconvulsive therapy (ECT), light therapy, and the like may also further define the subtype. Biofeedback data can also be used to determine which intervention works for the patient, and the subtype can be further specified based on responsiveness to specific intervention. For example, a bipolar patient may have a bipolar I disorder “without psychosis”, “with recurrent manic episodes” (if there were two or more episodes), “without rapid cycling”, “with seasonal affective disorder responsive to light therapy”, “with responsiveness to Lamictal”, “with money spending problem associated with manic episodes”, “with increased energy level during manic episodes”, “with history of engaging in risky business plans during manic episodes”, “with responsiveness to mindfulness exercise during depressive episodes”, “with elevated neuroinflammatory markers in CSF”, “with family history of manic depression responsive to lithium”, “currently in a manic episode”, “currently with sleep deprivation for 3 days,” and the like. By using these qualifiers in a unifying data structure to store the subtype, a large amount of information can be condensed to provide personalized medical care.

Additional criteria for bipolar disorder may include each of the DSM-5 criteria for diagnosis such as distractibility, elevated mood, increased goal-directed activity, irritable or expansive mood, increased energy, or prior hospitalization. Some of these data can be obtained directly from the patient via a questionnaire or an interview conducted by an AI avatar, for example, to clarify the diagnosis. A reduced need for sleep can be tracked by a biosensor provided in a wearable device worn by the user at night. The reduced need for sleep may indicate an onset of a manic episode. In the alternative, a peripheral device 110, 115 including a BIS strip, a portable EEG helmet, or a strip with a limited number of electrodes attached to the forehead can determine the sleep architecture of the user. Information such as a decrease in REM latency or sleep architecture instability can be used to further refine the diagnosis. For example, such findings can be assigned a point towards relevant diagnosis.

According to one embodiment, the subtype is stored in a data tree structure. The data structure may have binary, ternary, quaternary splitting, and the like at each node. According to one example, a binary tree node structure with binary splitting at each node may be utilized to store the subtype. Thus, a very detailed personalized medical diagnosis can be established and stored in a convenient manner. According to one example, such a data structure can be stored in a hardware memory, microchip, flash drive, memory key, circuits included in a patch, a skin tattoo with circuits or the like. The stored memory can be carried by a user, worn by the user, or implanted into the user's body for emergency use or in the event that the user wants to provide his subtype to a new clinician who does not know about the user's medical history. In another example, a patient security code can be carried by the user, worn by the user or implanted into the body of the user, and the security code can be used as an encryption key to discover the user's subtype in the event that the user becomes unconscious, such as in the setting of a brain injury.

The binary tree nodes may include a limited number of multi-splitting nodal levels, followed by binary splitting nodal levels. The binary splitting nodes may contain qualifier information regarding the presence or absence of certain phenotypes, endophenotypes, genetic information, family medical or psychiatric history, development history, prior psychological trauma, prior physical trauma, brain injury or stroke, brain imaging abnormalities, brain structural abnormalities, neuroinflammatory markers, comorbid diseases, comorbid symptoms or behavior (e.g. “increased money spending during a manic episode”), recurrence of episodes, state of remission, state of active disease, current disease state, and the like. An embodiment of a data tree structure is shown in FIG. 5.

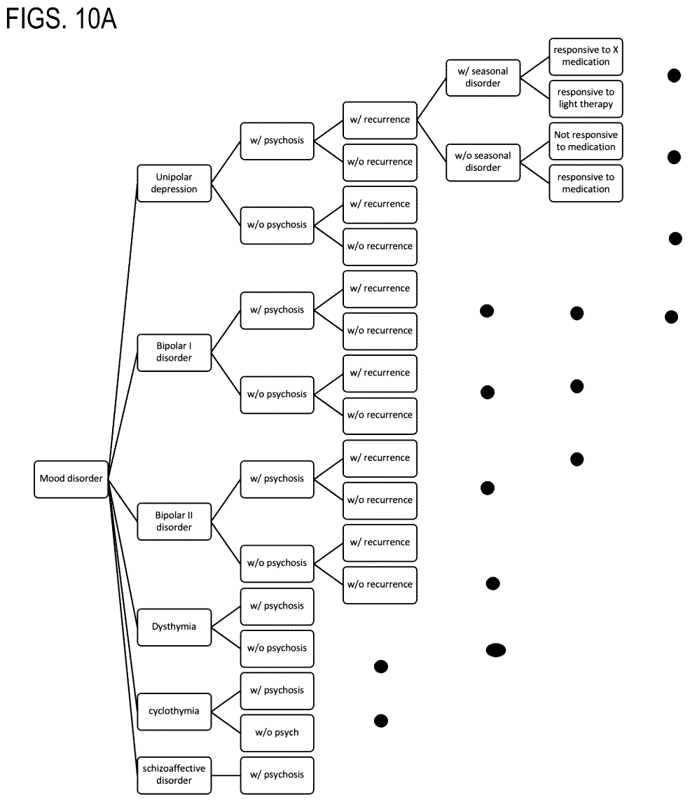

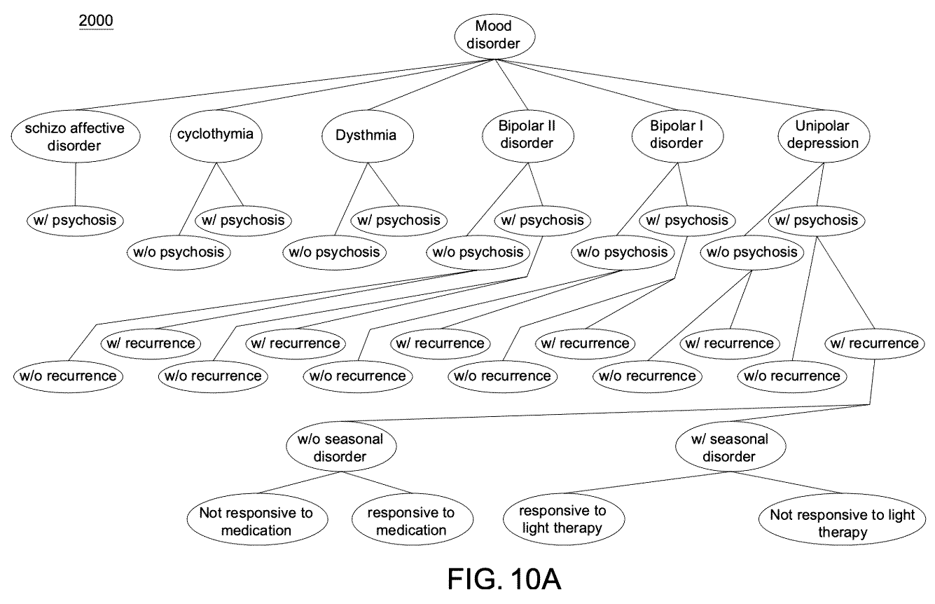

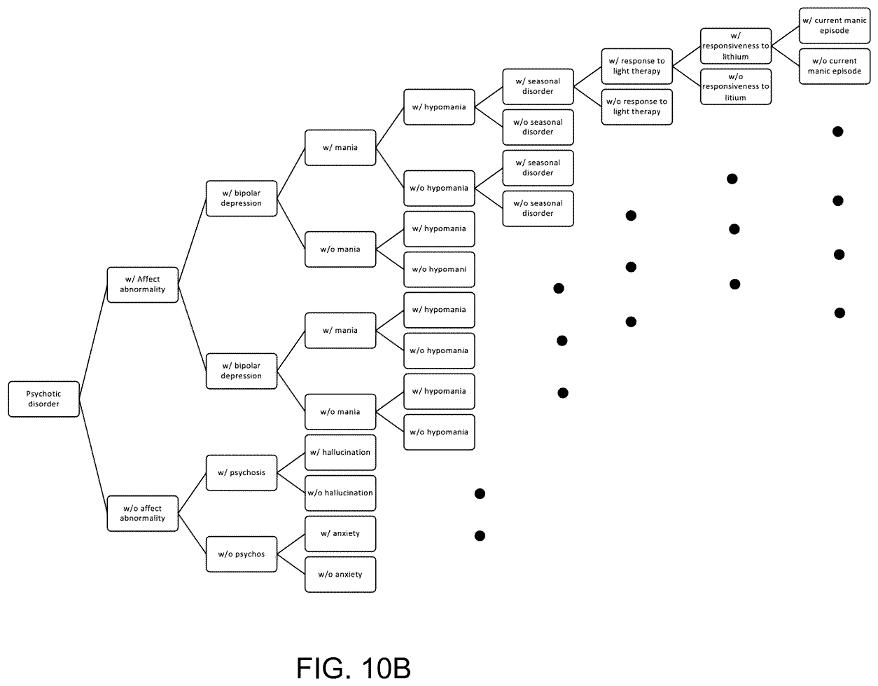

FIG. 10A depicts an example of a subtype data structure. Based on the illustrated example, by using the data tree structure, the current subtype of a patient can be easily updated by the assessment unit 300. Further, a large amount of information regarding the disease phenotype, endotype, genetic information, brain structural abnormalities, EEG abnormalities, intervention responsiveness, current symptoms, disease courses and the like can be effectively stored in a single data structure. Thus, the subtype can be easily retrieved by the intervention manager 800 in a dynamic manner to implement an intervention in response to a new clinical information. Further, by storing the current subtype in a data structure, the data structure can be easily transferred to other devices as a medical subtype token (MST). In addition, the data structure can be encrypted to protect the privacy of the patient. For example, the subtype data structure can be prepared into a medical subtype token (MST) by the MST token generator, and the medical subtype token can be transmitted from the user terminal 100 to a healthcare provider terminal 120 or a social support terminal 130 via the internet, via blue tooth, hard wire connection, or other data transfer method, without compromising privacy of the patient. The current subtype can be also rendered into a two dimensional (2D) image, three dimensional (3D) image or a four dimensional (4 D) video information in the form of a medical subtype visual token (MSVT) by the MST visual renderer for secure transfer between the user terminal 100, a healthcare provider terminal 120, a social support terminal 130, or as a part of the electronic medical record of the user in a hospital electronic medical record system. Given that the subtype data structure may be updated in the future, a fractal or fractal like 2D, 3D image or 4 D video rendering can be generated to facilitate transfer of the current subtype between different physical devices and electronic medical record system.

While a psychiatric disorder subtype was depicted as an example, the subtype data structure may include other medical disease information. For example, a subtype data structure can be built based on ICD-10 codes, to incorporate all known disease types, and other specific phenotype information and characteristics of the disease may be recorded into the subtype data structure. For example, a medical condition may branch to cardiovascular condition, to arrythmia, to atrial fibrillation, to atrial fibrillation that occurs 1-3 times per week, and to atrial fibrillation that is triggered by exertion, and to atrial fibrillation that is triggered by exertion 80% of the time when heart rate is above 130.

FIG. 10B depicts another example of the subtype data structure. The data structure is a tree node structure. More specifically, in this example, the data structure is a binary tree data structure composed entirely of binary nodes. A binary node can split to at most two other nodes. The splitting of the binary branches according to this embodiment may be based on the presence or the absence, the responsiveness or lack of responsiveness, or the efficacy and a lack of efficacy, for at least one of: a symptom, recurrence or lack of recurrence, affect disorder, psychosis, comorbid conditions, current remission or current symptom, current active episode or lack thereof, phenotype data, endophenotype data, genetic data, brain structural abnormalities, brain functional abnormalities, neuroinflammatory marker data, macromolecule and gene products in serum or CSF, responsiveness pharmaceutical agents, efficacy of interventions, and/or current symptoms. However, other examples of the data structure can be used. For example, the example shown in FIG. 10A is a tree node structure with multiple splitting at level 1. Ternary or quaternary nodes can increase the complexity of data structure. In either case, by using a tree node structure, a lot of information can be stored in one unifying data structure.

While a psychiatric disorder subtype was depicted as an example, the subtype data structure may include other medical disease information. For example, a subtype data structure can be built based on ICD-10 codes, to incorporate all known disease types, and other specific phenotype information and characteristics of the disease may be recorded into the subtype data structure. For example, a medical condition may branch to cardiovascular condition, to arrythmia, to atrial fibrillation, to atrial fibrillation that occurs 1-3 times per week, and to atrial fibrillation that is triggered by exertion, and to atrial fibrillation that is triggered by exertion 80% of the time when heart rate is above 130.

For example, the subtype data structure may include be directed to a medical condition, with the first node splitting to 5-20 medical condition criteria, such as cardiovascular, neuropsychiatric, musculoskeletal, endocrine, behavioral, rheumatologic conditions, reproductive disfunction, gastrointestinal, hepatic, hematologic issues, etc. Musculoskeletal condition may branch out to right upper arm, and then to right wrist condition, to carpal tunnel syndrome. Carpal tunnel syndrome can be further classified into additional subtypes such as triggered by exertion, certain rotation of the wrist, worse during night, improving with steroid injection, likely to flareup every day, every 2-3 days, once a week, once a month, etc.

According to this example, the current subtype can be easily updated due to the data structure, and a large amount of information regarding the disease symptom, phenotype, endotype, genetic information, macromolecule, brain structural information, EEG information, pharmaceutical agent responsiveness, intervention responsiveness, current symptoms, current biometric data, and disease course can be effectively stored in the tree architecture. The subtype can be easily retrieved by the intervention manager 800 to implement an intervention or a treatment in a dynamic manner.

Further, by utilizing this data structure on a large population, clinicians and researchers can get an insight regarding how psychiatric diseases should be better classified based on a set of characteristics, such as symptom, phenotype, endotype, genetic, molecular, brain wave, intervention responsiveness, and medication responsiveness. That is, it is now possible to reorganize the information based on parameters of the researcher's choice, such as responsiveness to a particular medication (lithium, for example) and the presence of certain endotype characteristic. By reorganizing the information, the researchers can easily obtain relationships between these parameters, such as correlation between the parameters, pathophysiology, and etiology of different symptoms.

The current DSM classification is based on phenotype-based information or symptoms as observed by psychological interviews, the presence or absence of depressive mood, manic episodes, psychosis, and the like. The DSM classification is not based on etiology, and provides very little information regarding pathophysiology, etiology, or effective treatment. For example, if a patient is determined to have a major depression based on DSM criteria, the criteria does not inform the clinician regarding whether the depressive mood has resulted from a traumatic brain injury, a childhood psychological trauma, a stroke in certain portions of the brain, abnormally small hippocampus due to genetic factors or childhood trauma, or due to genetic disposition. A clinician may blindly try out a medication, such as fluoxetine for four weeks, only to discover that the patient does not response to fluoxetine. However, if we can establish a classification system that incorporates the fact that the patient had a stroke in a specific area of the brain based on an MRI or that the patient biologically has small hippocampi, we can more readily predict whether fluoxetine will work on a specific patient by comparing data from other patients having a similar subtype.

Thus, by applying the device of the present disclosure to a large population of patients, such as 100 patients, 1000 patients, 10000 patients, or more in a large study, clinicians and researchers can elucidate the correlation and even the cause-and-effect relationships between various parameters such as the presence or absence of structural abnormality, reduced hippocampal size, presence of a gene or the like and the efficacy of a specific pharmaceutical or intervention. The extracted information can be used to come up with an alternative classification system for a psychiatric disorder. Further, the extracted information can be used to establish new qualifiers so that the subtypes can be better defined.

According to one embodiment, the remote management system 400 can update the assessment algorithm based on the extracted information, as well as update the available subtypes and alert criteria. This allows new findings and knowledge regarding psychiatric disorders and treatments to be seamlessly incorporated into the device 10.

According to one embodiment, the MST token generator prepare the current subtype information into a medical subtype token (MST) for easily transferred to a remote device. The MST can be encrypted to protect the privacy of the patient. For example, the encryption can be performed based on a dynamically changing key originating from the remote management system 400. The medical subtype token can be transmitted from the user terminal 100 to a healthcare provider terminal 120, a social support terminal 130, or to a medical record system via the internet, via blue tooth, hard wire connection, or other data transfer method, without compromising privacy of the patient. The MST visual renderer may render the current subtype into a 3D image or a 4 D video information for secure transfer between the user terminal 100, a healthcare provider terminal 120, a social support terminal 130, or as a part of the electronic medical record of the user in a hospital electronic medical record system. Given that the subtype data structure may be updated in the future, a fractal or fractal like 3D image of 4 D video rendering can be generated to facilitate transfer of the current subtype between different physical devices and electronic medical record system.

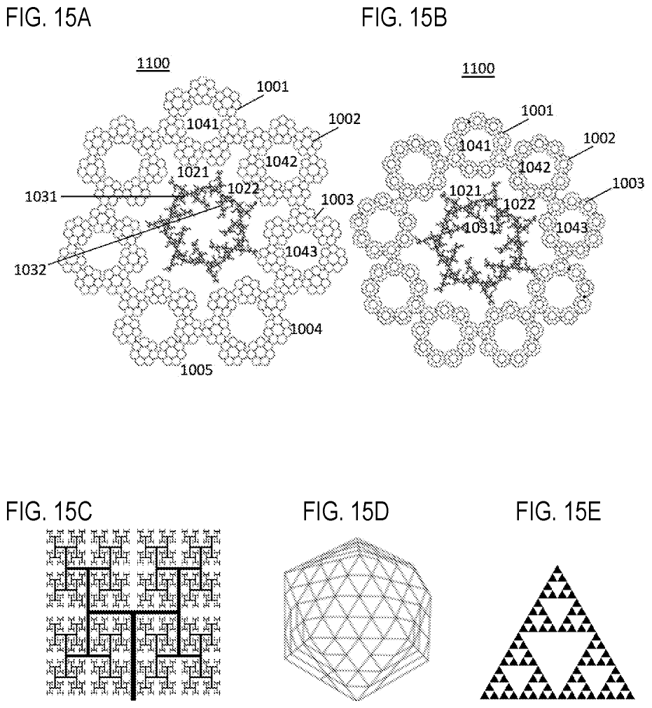

FIG. 15 depicts examples of medical subtype visual tokens. Fractals or repeated patterns are utilized so that change in subtype database structure can be accommodated. The medical subtype visual tokens 1100 in FIG. 15(A) and FIG. 15(C) illustrates facture structure can be utilized to expend the data structure. The first wheel of the medical subtype visual token 1100 in FIG. 15(A) includes seven leaflets 1001, 1002, 1003 . . . 1007, while the first wheel of the medical subtype visual token 1100 in FIG. 15(B) includes nine leaflets 1001, 1002, . . . 1009. The first leaflet 1001 may be used to indicate when the token is created, encryption information, and/or the type of medical classification that is used, for example, framework based on ICD-10, ICD-11, DSM-5, DSM-6, etc. Leaflets 1002, 1003, 1004, etc. can be used to indicate various medical issues that patient has. For example, if the patient has a mental health diagnosis, leaflet 1002 can be used to indicate the diagnosis. If the patient has cardiovascular issues, leaflet 1003 can be used, so forth. The subtype of major diagnosis can be expressed in the middle of the hub, using fractal structures of 1021, 1022, 1031, 1032, etc. The spaces 1041, 1042, etc. provided within leaflets 1001, 1002, etc. can be used to describe subtypes of respective diagnosis. Leaflet numbers can be increased based on complexity of patient's medical issues and expansion of subtype structure. FIGS. 15(C) and (D) illustrate different fractal organizations that may be utilized. The medical subtype visual token can be captured by a camera of a remote device to transfer the subtype information of a patient. FIG. 15(D) provides a 4 D rendering. Each facet of the 3D polygonal structure can include, for example, a triangular fractal structure such as shown in FIG. 15(E) to embed information. The polygonal structure can be rotated to produce video information that may be captured to transmit the medical subtype information to another device having a camera. Information can be embedded by filling out a polygonal space, placing a dot at an appropriate location, or creating a void or a white space where a positive image would be otherwise present. The data can be embedded in a digital 3D polygonal structure as shown in 15(D), and the polygon may be rotated in view to transmit appropriate information to a receiving device. The receiving device may include a camera with lenses and photosensors to capture the image, and a processor that processes the image to extract the medical subtype information. The processor may be a part of the data collection unit of the receiving device. The extracted subtype information can be stored in the assessment unit of the receiving device. The processor may access the remote management system 400 to determine the assessment algorithm used to create the medical subtype information. The extracted subtype information can be used in the receiving device to generate intervention, using the intervention manager 800. Other units of the receiving device include that described with respect to FIG. 1, which are a part of this disclosure and will not be repeated here.

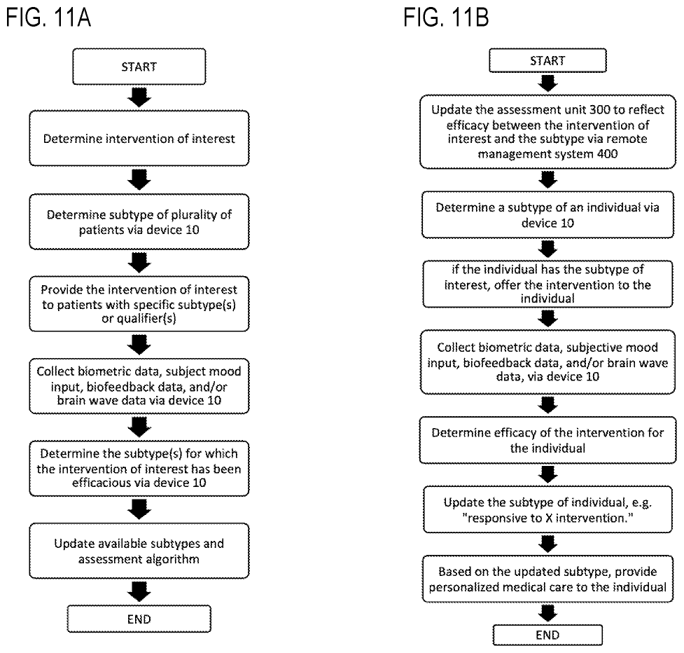

FIG. 11A depicts an example of a method of determining the relationship between an intervention of interest and a subtype. For example, the relationship may be correlation or cause and effect between the intervention and the subtype, of the efficacy of the intervention for the subtype. FIG. 11B depicts an example of a method of providing personalized medical care based on a subtype.

The method of determining the relationship between a subtype and an intervention of interest includes the step of: determining intervention of interest; determining subtype of a plurality of patients, providing the intervention of interest to patients with specific subtype(s) or qualifiers(s); collect biometric data, subjective mood input, biofeedback data, and/or brain wave data via device 10, determining the subtype(s) for which the intervention of interest has been efficacious via device 10, and updating available subtypes and assessment algorithm.

For example, the plurality of patients may comprise 100 or more, 1000 or more, or 10000 or more patients. Alternatively, the number of patients studied may be that which is required to achieve a confidence interval of 90% or more, or 95% or more, to statically establish the efficacy. In another example, a smaller sample size may be acceptable if each of the patients have a specific qualifier or subtype. For example, five patients having the subtype “bipolar I disorder w/seasonal affective disorder responsive to light therapy” can be considered a sample size test a hypothesis because it is not easy to find multiple patients with the specific subtype, “bipolar I disorder with seasonal affective disorder responsive to light therapy.” For example, the sample size may be 5 or more, 10 or more, 20 or more, 100 or more, 1000 or more. For a small study, the sample size may range between 5 or more and less than 10000, or 50 or more and less than 1000. Alternatively, the sample size may be calculated based on the number of patients required to establish 90% or more, or 95% or more statistical confidence.

An intervention of interest can be provided to the patient population as a part of a clinical study or as a part of normal clinical treatment to relieve the patients' suffering. The device 10 can monitor or collect various biometric data, subjective mood, mood chart, biofeedback data, neurofeedback data, and/or brain wave information from the plurality of patients. The collected information can be sent to a remote management system 400 to determine the presence of statistically significant correlation between the intervention of interest and the subtype. For example, the remote management system 400 may try to determine whether there is 90% or more, or 95% or more of statistical confidence, or the remote management 400 may try to determine whether at least a set percentage of patients with specific subtype has responded positively to the intervention or treatment.

Once the correlation between a subtype, such as “bipolar I disorder with seasonal affective disorder” and the efficacy of an intervention of interest, such as “noon-time light therapy” are established, the remote management system 400 can use this information to update the assessment algorithm of many other devices 10 that belong to other patients. Thus, according to one example, all of the patients having the subtype “Bipolar I disorder with seasonal affective disorder” who use device 10 may now automatically be offered “noon-time light therapy” as an intervention by device 10. If the intervention comprises pharmaceutical intervention that requires an approval from a clinician prescriber, the alert for the intervention can be sent directly to the clinician prescriber of each patient.

After updating the assessment algorithm for multiple devices 10 by a centralized system, such as the remote management system 400, it now becomes possible to provide with confidence the specific intervention, for example “noon-time light therapy” to individual patients who has the diagnosis of “bipolar I disorder with seasonal affective disorder.” The intervention manager 800 of an individual patient can offer this intervention to the individual. Thus, based on this embodiment, it is now possible to deliver a truly personalized medical care to an individual by seamlessly integrating new clinical findings, such as the efficacy of an intervention for a specific subtype, based on information gathered from a plurality of patients.

For example, let's presume a scenario with a new user of the device 10 for dynamically determining psychiatric disorders and providing intervention based on the determination. Joe may have been newly diagnosed with a manic episode and offered to use the device 10 for better management of his condition. After charting the mood input from Joe for 6 months, the assessment unit 300 may discover that Joe's mood plummeted in early November and remained low for several weeks. Based on the new information, the assessment unit 300 may determine that Joe may be a bipolar I disorder patient “with a possible seasonal affective disorder.”

It may be that a population study of a plurality of patients (e.g. 2000 or more patients) who were using device 10 have recently shown that there is a statically significant improvement in the mood symptoms for patients having Joe's subtype when they were provided with noon-time light therapy. This new discovery may have been incorporated to the algorithm of the assessment unit 300 of many other patients.

When Joe's device 10 dynamically determines Joe may possibly have seasonal affective disorder, the device 10 can now automatically offer the noon-time light therapy or alert Joe's clinician to offer this therapy. If Joe accepts the offer and tries out the noon-time light therapy, the device 10 can monitor the mood input of Joe as well as other biometric data while the light therapy is delivered to Joe over the next several days. If Joe's mood improves during the trial, it is likely that Joe is a person who is individually responsive to the noon-time light therapy. Accordingly, Joe's subtype can be updated to “Bipolar I disorder” “with seasonal affective disorder” “responsive to noon-time light therapy.” Based on the new subtype assigned to Joe, the assessment unit 300 and intervention manager 800 can now confidently alert Joe's clinician and consistently offer the noon-time light therapy to Joe, thereby providing a personalized medicine based on Joe's own specific responsiveness to light therapy. Thus, it becomes possible to provide a personalized care to Joe even with a limited involvement of the clinician.

FIG. 11B depicts an example of a method of providing personalized medical care. The method of providing personalized medical care may include the steps of: updating the assessment unit 300 to reflect efficacy between the intervention of interest and the subtype via remote management system 400; determining a subtype of an individual via device 10; if the individual has the subtype of interest, offering the intervention to the individual; collecting biometric data, subjective mood input, biofeedback data and/or brain wave data via device 10; determining efficacy of the intervention for the individual; update the subtype of individual based on the efficacy on the specific individual. For example, the new qualifier for the subtype may be “responsive to X intervention.” Joe may be determined to have a “bipolar I disorder” “with seasonal affective disorder” “responsive to noon-time light therapy.” Based on the updated subtype, the device 10 can continue to provide the individual with personalized medical care based on the patient's own responsiveness to an intervention, even with minimal involvement of the individual's physician. The device 10 may also generate a report or send an alert to the individual's physician so as to inform the physician that the specific patient is responsive to a particular intervention. The physician can use such report to provide a better care for the patient.

This method can be also applied to the responsiveness of certain population of patients to a specific pharmaceutical agent. For example, patients who has history of stroke can be explored for their responsiveness to an SSRI. If it is determined that these patients don't respond to SSRI, then clinicians do not need to waste the time of trying out an SSRI for four months just because a patient has depression. On the other hand, if a determination is made based on a large population that an intervention works for a specific patient subtype, the device 10 can alert prescribers and make it possible to deliver the personalized medical care to patients much more quickly, and without limited involvements of the physicians.

For example, in the past, clinical studies were conducted by researchers on a limited number of patients to determine the efficacy of an intervention in various educational institutions and private entities throughout the world. These institutions had no way of efficiently sharing patient information with one another. Further, these institutions have different ways of characterizing their patient populations and devising details of an intervention they are trying to study. There was no standardized way to classify the population being studied nor to provide an intervention. Thus, research often produced contradictory results. More than that, it usually takes several months to publish a new finding in a paper journal, and it takes several months after the publication before the general public and the majority of clinicians become educated about the result of new research. Since there is no standardized way of exchanging information, clinicians are often faced with contradictory research results or feel that they do not have enough evidence to offer a new treatment to their patients. This causes clinicians to be hesitant regarding offering a new treatment. As a result, it usually takes years for a new clinical discovery to be implemented in patient care. The method disclosed herein is expected to speed up the development of new treatments, and the new treatment can be tried out and delivered in a personalized manner to patient in a more effective manner, with limited day-to-day involvement of clinicians.

According to an example of this method, the remote management system 400 may update the assessment algorithm of many devices 10 in order to reflect the efficacy of a new intervention for a subtype of interest based on a large population study. Thereafter, the device 10 owned by individual patients can dynamically offer the new intervention if the individual patient is determined to have the subtype of interest.

In response to a determination that the specific patient has the subtype of interest, the device 10 may automatically offer the intervention to the specific patient or suggest the intervention to the clinician for approval. The device 10 can also monitor or collect biometric data, subjective mood input, biofeedback data, brain wave data and/or answers to questionnaires, to determine the efficacy of the intervention for a specific patient. The device 10 can then establish the efficacy of the intervention for the specific patient based on the monitoring the specific patient and collecting data regarding the response of the specific patient to the intervention. If the intervention is determined to be efficacious, the device 10 updates the subtype of patient to reflect the finding that the specific patient is “responsive to” a specific intervention. Thus, by updating the subtype of the patient based on responsiveness, the device 10 can provide a personalized medical care to the specific patient with increased confidence.

Further, the assessment unit 300 may be provided with a detection unit for detecting subtype change based on newly received biometric data or other information collected by the data collection unit 200. The updating of the current subtype of the disease usually corresponds to a clinically significant finding. Thus, the contemporary alert initiator may be each time triggered in response to such a subtype updating event. The contemporary alert initiator can prompt the intervention manager 800 to dynamically provide a patient alert, a healthcare provider alert, or a social support alert. The intervention manager 800 can also offer an intervention such as a virtual therapist (an avatar or an AI speech recognition device for carrying out conversations), a music therapy, a mindfulness exercise, a light therapy, and the like. The intervention manager 800 can work with the data collection unit to monitor the biofeedback response of the patient in conjunction with providing these therapies, thus determining whether the patient is responsive to a specific therapy. A list of responsive therapy can be stored in the data collection storage 600, and the subtype can be updated to indicate responsiveness. For example, a patient may be determined to have a bipolar I disorder, with psychosis, with seasonal affective disorder, with mania episode onset around November of each year, and with responsiveness to noon-time light therapy based on past biofeedback data, and with recurrent manic episodes and depressive episodes in the past, and with current depressive episode. This specific current subtype can be stored in a designated memory space of the assessment storage 700 by the assessment unit 300 in the format of a data tree, with each nodal splitting presenting the presence or absence of additional qualifiers. The current subtype can be updated based on crucial newly received information in the future, in a dynamic manner.

According to one embodiment, the data collection unit 200 may collect sleep pattern, sleep architecture, movement information collected from an accelerator which may reveal information regarding akathisia or extrapyramidal symptoms, location information from GPS that may provide insight into traveling or increased activity, financial information such as online shopping or screen time, brain wave related information obtained through full 10-20 EEG electrodes, a portion of EEG electrodes placed on the forehead as a patch or headband, or a BIS strip, speech related information such as speech rate, volume, rhythm, content based on speech recognition software and speaker, heart rate related information or pulse pressure from biosensor of a smart watch or smart band, temperature related information from peripheral device, answers to questionnaires regarding mood or diagnosis clarifying questions according to DSM manual, the level of lithium, Depakote or other medications as determined by a peripheral device or manually entered to the computer or portable device, the use of substance such as cocaine, PSP, LSD, marijuana, alcohol based on the user's input or based on urine toxicology information, time stamps related to information receipt, and the like.

Accordingly to one embodiment, the device 10 may monitor any combination of the following parameters to determine or update a user's subtype: mood symptoms such as depressive mood, hypomania or hypermania; abnormal sleep architecture, such as reduced total sleep hours, decrease in REM latency, change in sleep architecture, early awakening, difficulty falling asleep, delay in going to sleep (circadian rhythm shift), and average sleep hours; presence of comorbid disorders such as seasonal affective disorder, eating disorder, panic attack; behavioral symptoms such as increased activity, increased money spending, irritable voice, pacing around, and the like; vitals such as fast heartbeat, elevated blood pressure; metabolic syndromes which may be side effect of medication; presence of psychosis such as paranoid delusion provided in a questionnaire, excessive traveling detected by GPS; fluctuation in subject mood detected by mood input; family history of similar condition; responsiveness to medications and other interventions; and the like.

Dynamic detection of sleep pattern allows for early detection of the disease. The average sleep hours can be calculated daily or weekly based on the length of sleep from last 30 days for example. The average sleep hours can be also calculated from a range of time between 5 days to two months, or from one month to six months. A calculation can be made from last year's data based on the current season or month. For example, the sleep pattern of November from 2022 can be compared to the sleep pattern from November of 2023. A calculation can be made as to whether there is more than 20% change in total sleep hour from the average sleep hours during the last one week, two weeks, three weeks, four weeks and the like. A reduction of more than 20% of sleep length compared to the average sleep length can initiate an alert that the patient may be developing a possible manic episode. A calculation can also be made as to whether there is more than 30% change in total sleep hours during the last two to three days when compared to the average sleep length over the last week, for example. The degree of change may allow the assessment unit 300 to determine the severity of sleep deprivation and the possible risk of developing a manic episode. Sleep hour monitoring is an important clue to the onset of manic episode, depressive episode, and psychosis. The result of sleep hour monitoring can be used by the intervention manager 800 to notifying the patient, caretaker, social support, clinician, social worker and the like to help the patient get more sleep and get timely treatment for the new symptoms.

Seasonal affective disorder is a common comorbid disorder for bipolar disorder patients and patients with depression. The change in sleep pattern around November, or at the time of shortening of day light, can be detected by sensors that detect movement and heart rate of users, or by detecting EEG patterns, REM sleep pattern, and the like.

Subject mood input can be provided by the user. The user may be requested to select his/her mood from a display of emoji on a touch screen. In the alternative, an avatar can ask the question how the patient feels. The patient's face can be detected by a camera and provide further information regarding how the patient feels. Such a camera can be also used to detect movement disorders in the face, such as eye blinking due to tardive dyskinesia.

The device 10 can ask the user's permission to keep track of various behaviors of the patient, including the patient's pattern of eating (important for metabolic syndrome due to antipsychotic use, for anorexia and other eating disorder), pattern of shopping and money spending (possible symptom of manic episode), travel and substance use (possible symptom of manic episode), and the like. The device 10 can ask the user's permission to track the funds in the user's bank account to make sure the patient is not overspending money during a manic episode. The device 10 can ask the user to monitor screen time or time spent in online shopping. A bipolar patient may voluntarily submit to such monitoring because they realize that they need a confidential counseling when they engage in such a behavior. The intervention manager 800 can provide the user with a reminder to reduce money spending or to not to engage in big purchases, for example. The device 10 can also offer calorie counting or may be able to monitor bingeing and purging. The device may receive weight information from a peripheral device and determine the patient's BMI, or use a biosensor to detect the glucose level, heart rate, body fat percentage, and the like, to ensure the patient is maintaining healthy eating. The device can also track the location of the patient using a GPS, or use an accelerometer to detect a patient's movement. Some bipolar patients and psychotic patients engage in excessive travel due to grandiose ideas or paranoia. A manic patient may pace around the house or make excessive movements that is concerning for the patient's abrupt mood change. The location monitoring can be used to determine the user's behavior, and even to help the family members and clinicians locate the patient.

The device 10 can also use vitals such as heart rate, breathing rate, average heart rate over an hour, average heart rate over a week, average heart rate over one month to six months, and the like to determine the user's emotional state. A manic or irritable patient may exhibit elevated heart rate. The heart rate also shows the patient's activity level. People's heart rate falls during their sleep, indicating whether they are getting sufficient rest. A psychotic patient may not sleep at night and remain anxious due to paranoid thoughts. Their cortisol level may be elevated to due worries and anxiety brought on by their delusional thought content. Thus, vitals provide additional information regarding the state of the patient.

Vitals can be used to provide biofeedback or measure the responsiveness of the patient to a therapeutic intervention such as listening to calm music or taking a digital meditation session. The responsiveness can be reflected in subtype as “responsive to music therapy during episodes of panic attack” or “responsive to meditation during active psychosis.” Many genres and special frequency music can be tried on a patient to determine the most calming music for the particular patient. An EEG monitoring or a brain wave monitoring can be also used to provide a neurofeedback training, or help the patient learn to calm down or to increase concentration.

Inspirational messages based on religion, spiritual content, and the like can be recorded and provided with biofeedback. It is noted that these therapeutic interventions can be helpful to patients with mild mood disorder but can be a source of confusion to patients with severe psychosis or excessive superego pathology. A questionnaire can be utilized to determine whether the patient has an over-active superego, delusions related to religion, and the like to screen out patients who may negatively respond to such a content. Also, an input from the clinician can be used to determine the level of function that patient has and whether the patient is currently receptive to such a therapy. EEG and brain wave monitoring can be combined with vital monitoring to determine the optimal intervention for a patient.

A microphone can be used to detect the rate of speech, irritability sensed by pressured speech, and in voice changes. Pressure speech is a characteristic of a manic episode. Patient may increase the rate of speech, the quantity of speech, and the tone (pitch, amplitude) of speech. Family members may notice the change and provide an input to the device, or the patient can answer a questionnaire. Further, an artificial intelligence-based software can detect the change in the rate of speech and can alert the patient or healthcare professional of the possible onset of a manic episode.

For recurrent manic episodes, it is possible to determine cycling speed, depression length, depression speed. The device may also use questionnaires to determine the mood state of the patient, such as depression, anxiety, mania, overvaluation, confidence. Vital monitoring and EEG or brain wave monitoring, as well as behavioral tracking such as money spending can be used to also determine efficacy of intervention, including medications and therapy.

Traditionally, psychiatrists have been taught not to give an antidepressant to a bipolar disorder patient because the antidepressant may cause a manic episode or cause a worse manic episode. Because bipolar patients often experience severe and extended periods of depression between manic episodes, many of the patients suffered greatly during the depressive episodes without the aid of antidepressants that are available to patients with unipolar depression. Many bipolar disorder patients have taken actions during their depressive episodes to commit suicide. Because the present device makes it possible to rapidly detect the onset of a manic episode, it becomes possible to deliver an appropriate amount of fast acting medications to target the current mood to be euthymic and quickly determine when to stop an antidepressant. Otherwise, the patient will remain on an antidepressant until the next appointment during which he or she will be evaluated by a psychiatrist. This has been a problem because the patients' next appointment with the psychiatrist may be in two months or three months. By this time, the patient could have developed a new manic episode prior to being evaluated by a psychiatrist. If an antidepressant is given to help the patient with a depressive episode without frequent monitoring, the patient can indeed develop a manic episode and get hospitalized or make a lifetime mistake before the next appointment. By using the monitoring device, it is now possible to detect the onset of a manic episode early and stop the antidepressant. Since each medication needs to be stopped in a different manner, the appropriate protocol for stopping an antidepressant can be assessed in advance by the psychiatrist, and provided to the patient with appropriate reminder via the device.

Some agents that have been used to elevate mood in bipolar patients include bupropion (low risk of withdrawal), trazodone (moderate risk of withdrawal with added benefit of promoting sleep), Latuda (lurasidone HCl, FDA approved for bipolar depression), and the like. Close monitoring for the development of manic episode allows psychiatrists to better treat the depressive episodes in bipolar patients. Further, patients can be directly alerted to get more sleep, to contact psychiatrists for sleep medication, and the like.

Many bipolar disorder patients need a life-long monitoring of their symptoms. With the right management strategy, many can live a fulfilling life. However, the medical cost for receiving ideal care is often prohibitively high. By using the device 10, a long-term monitoring of bipolar disorder and dynamic subtype determination can be carried out continuously for timely intervention. For example, the device 10 can monitor: sleep pattern; seasonal affective disorder; pattern of eating, shopping, money spending, traveling; change in voice such as pressures speech, talking to face, irritability; fluctuation between depressive mood and manic mood and cyclic speed, depression severity, presence of psychosis; frequent mood monitoring by obtaining mood input directly from the patient and generating a high-level analysis; efficacy of various treatment modality; and the like. Further, the device 10 can provide timely patient education and counseling. For example, the device 10 can recommend a patient in a manic episode to get more sleep, stop spending money, avoid making rash decisions, and to contact the psychiatrist or family members for support. An avatar may carry on a conversation with the patient, “You have been sleeping very little for the last three days and you have been spending an average X dollars per week online. I am concerned that you are developing a manic episode. How about catching on your sleep tonight? Do you want me to send a notification to your psychiatrist so he can send you a prescription for sleep medication?” Many patients will appreciate getting a timely evaluation of their symptoms so that they can better manage their psychiatric condition.

Further, by monitoring biometric data, the assessment device can determine whether a patient is developing a side effect such as serotonin syndrome, lithium overdose, medication withdrawal, alcohol or substance withdrawal, or abnormal movement due to antipsychotic use, metabolic syndrome, and the like. Lithium overdose and serotonin syndrome can exhibit hypomania, restlessness, ataxia, tremor, and the like. Neuroleptic malignant syndrome can cause fever, altered mental status, muscle rigidity, blood pressure fluctuation. Some antipsychotic medications can cause QTc prolongation, and the use of EKG prior to the first administration is a standard protocol at most hospitals. Symptoms of medication withdrawal can include flu-like symptoms, insomnia, nausea, imbalance, sensory disturbances, hyperarousal, sweating. Further, different antidepressants can cause different symptoms, such as the abrupt cessation of TCAs may cause cholinergic-rebound phenomena, flu-like illness, myalgia, and abdominal cramps. Antidepressants like mirtazapine can cause antihistaminergic activity that can cause an antihistaminergic rebound symptoms, such as insomnia, agitation, anxiety. Alcohol and benzodiazepine withdrawal can cause a dangerous fluctuation in blood pressure, sweating, anxiety, seizure and the like. These side effects or withdrawals can manifest as changes in heart rate, blood pressure, body temperature, movement, patient's subjective mood change provided by the patient, level of sweating, change in sleep pattern, and the like. Biosensors can now detect the perspiration, blood pressure, blood pressure fluctuation, heart rate, sleep pattern, tremor, body temperature, and the like, and can obtain EEG and EKG to determine the occurrence of seizure and the prolongation of QTc.

Occasionally, fast acting medications with low side effect profile are important to deal with acute psychiatric conditions. For agitation in an inpatient unit, parental medications are often used. Some patients with schizophrenia require long-acting injectable medications to control their symptoms.

Parenteral medications refer to medications that are designed for subcutaneous (fatty tissue under the skin), intramuscular (in a muscle), intravenous (in a vein); and/or intrathecal (around the spinal cord, to CSF) administration. Most commonly used long-acting injectable medications belong to the category of intramuscular injections. However, Perseris is a subcutaneous formula of risperidone that can be delivered once a month. Most patients visit a clinician to receive their IM injection because most lay people are not trained to give themselves an IM injection, and further because the clinicians would like to observe patients for any dystonia or adverse medication side effect. The device can be provided with a parenteral medication administerer 910 that can automatically deliver these medications, and can provide the monitoring necessary to determine whether the patients are having abnormal movements from medication administration.

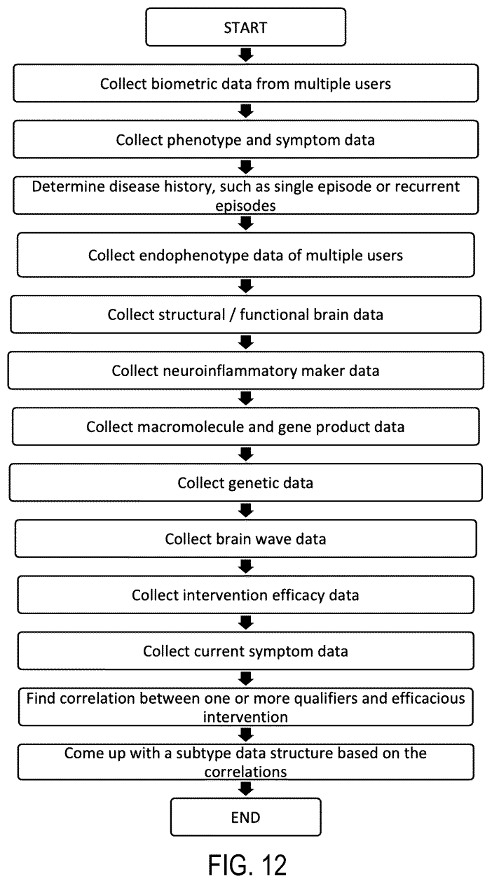

FIG. 12 depicts an example of a method of classifying psychiatric disorders into subtypes. The method of classifying psychiatric disorders may include the steps of collecting biometric data of multiple users via wearable devices over prolonged length of time, the prolonged length of time comprising: one day, one week, one month, one year, 5 years, and/or 10 years or longer. Biometric data may include heart rate, blood pressure, rate of speech, facial expression, movement velocity, angular rotation, blood glucose level, electrical activity of brain/muscles/heart, physical location, and the like. The method may involve determining phenotype data of the multiple users over prolonged time. The phenotype data may include, for example, the presence or the absence of a symptom, the symptom comprising at least one elected from the group consisting of depression, mania, hypomania, psychosis, delusion, hallucination, or a combination thereof. User interaction with an AI character projected on a touchscreen of an electronic device may be used to obtain answers to some questions regarding the user′ phenotype. Questions regarding DSM-5 criteria may be collected from the user via questionnaire, AI character, AI avatar, and the like.

The method may involve collecting endophenotype data of the multiple users over a prolonged length of time. The endophenotype data may include the presence or the absence of characteristics that provides a link between phenotype, disease manifestation, and gene and possible etiology of the disease. The endophenotype may include at least one elected from the group consisting of sleep architecture abnormality, sleep length abnormality, seasonal affective disorder, dysregulation of motivation and reward, dysregulation of emotional reactivity, impaired facial expression recognition, attention and concentration dysfunction, executive dysfunction, impulsivity dysfunction, and suicidality or suicidal ideation, or the like, any combination thereof.

The method may involve collecting brain structural data and brain functional images of the multiple users. The brain structural data may be detected by MRI, CT or other structural medical imaging. The data may include the presence or the absence of white matter abnormalities, anterior cingulate cortex abnormalities, volume reduction in the anterior cingulate cortex, volume reduction in the anterior cingulate cortex ventral and anterior to the genu of corpus callosum, volume reduction in left subgenual anterior cingulate cortex, brain connectivity abnormality (such as detected by fMRI or functional medical imaging), brain structural abnormality (such as detected by MRI, CT or other structural medical imaging), or a combination thereof. Overtime, more discoveries will be made regarding structural abnormalities or alterations that predispose patients to psychiatric disorders. Based on the present method, new findings can be easily incorporated to the subtype data structure by the remote management system 400.

The method may involve collecting brain functional data of the multiple users, the brain functional data having been detected by fMRI such as blood-oxygen-level-dependent imaging (BOLD) or regional homogeneity (ReHo), positron emission tomography (PET) or other functional medical imaging. The brain functional data may include the presence or the absence of: hypoactive brain area; hyperactive brain area; hypoactive brain network connectivity; hyperactive brain network connectivity; a decrease in network connectivity over the entire brain; decreased ReHo in a specific part of the brain, such as the frontal lobe, parietal lobe, anterior cingulate gyri, posterior cingulate gyri, occipital lobe, and/or cerebellum; increased ReHo in a specific part of the brain, such as parahippocampus, thalamus, hypothalamus, and/or striatum; altered activity detected in medial frontal and anterior cingulate gyri; decreased fronto-temporal white matter functional activation, and/or a combination thereof.

The method may involve collecting neuroinflammatory marker data of multiple users, the neuroinflammatory marker data including the presence or absence of abnormalities in neuroinflammatory markers or an elevated or reduced level of the neuroinflammatory markers. The neuroinflammatory markers may be at least one of IL-10, IL-6, TNF, cytokines, osteoproteigerin (OPG), C-reactive protein (CRP) or a combination thereof, found in serum, plasma, and/or CSF.

The method may involve collecting macromolecule and gene product data of the multiple users. The macromolecule and gene product data may include the presence or absence of abnormalities in cerebral glutamate level, CCL11, sTNFR1, CCL24, CXCL10, BDNF, CRP, TWEAK, IL-10, OPG, C-reactive protein (CRP), and the like, and a combination thereof.

The method may involve collecting EEG signal data of multiple users. The EEG signal data may include the presence or absence of abnormalities in sleep architecture, reduction in REM latency, pattern deviation from baseline pattern taken while the patient was awake or asleep, or a combination thereof. The EEG signal data may be a full international 10-20 EEG recording, a 10-10 recording, or limited to data received from a limited number of electrodes placed on the forehead of the patient, such as 4 electrodes, 6 electrodes, 8 electrodes. The number of electrodes may range between 2 to 20 electrodes, or 4 to 14 electrodes. According to one example, the EEG signal data may be organized into a BIS index.

The method may involve collecting genetic data from the multiple users. The genetic data may include brain connectivity abnormality (such as detected by fMRI or functional medical imaging), brain structural abnormality (such as detected by MRI, CT or other structural medical imaging), abnormal proteins, cerebral glutamate level, abnormal EEG signal, or a combination thereof.

The method may involve collecting clinical treatment responsiveness data from the multiple users. The clinical treatment responsiveness data may include the responsiveness or lack of responsiveness of: antidepressant (SSRI, SNRI), antipsychotic (first generation antipsychotic, second generation antipsychotic), mood stabilizer (lithium, second generation antipsychotic), anticholinergic agents (benztropine, Benadryl), sleep aid (melatonin, trazodone, Ambien), or a combination thereof.

The method may involve collecting intervention efficacy data from the multiple users. The intervention efficacy data may include the efficacy or lack of efficacy of: light lamp therapy for seasonal affective disorder, music therapy, biofeedback therapy, neurofeedback therapy, virtual avatar counseling, psychosis voice hallucination avatar training, mindfulness exercise, or a combination thereof.

The method may involve collecting current symptom data from the multiple users. The current symptom data may include the presence or absence of: current manic episode, current depressive episode, current suicidal ideation, current suicidal plan, current homicidal ideation, current agitation, current irritability, current violence potential, current risk for elopement, current code strong, current hospitalization, current psychosis, current paranoia, current idea of reference, current auditory hallucination, current delusion, current extra-pyramidal symptoms, current tardive dyskinesia, or a combination thereof.

The method may involve determining the psychotic disorder subtype of each of the multiple users, wherein the subtype is stored in a binary tree structure indicating the presence or the absence, the responsiveness or lack of responsiveness, or the efficacy and a lack of efficacy, for at least one of: a symptom, a phenotype data, an endophenotype data, a brain structural data, a brain functional data, neuroinflammatory marker data, macromolecule and gene products data, pharmaceutical agents, other interventions, and/or current symptom data.

The method may include determining association or correlation between pharmaceutical agents and the psychotic disorder subtype of each of the multiple users.

The subtype data structure may include DSM diagnosis such as bipolar I disorder, bipolar II disorder, unipolar depression, generalized anxiety disorder, schizoaffective disorder, schizophrenia, mood associated psychosis, psychosis elicited by another medical condition, affect disorder elicited by another medical condition, and the like. In addition, qualifiers can be provided in a binary structure regarding the presence or absence of certain phenotype, endophenotype, genetic predisposition, structural or functional brain image data, neuroinflammatory marker data, macromolecules and/or gene product presence in serum/plasma/CSF, brain wave data including EEG or BIS index, the presence of current symptoms, and the like. The subtype may be stored in a binary tree structure.

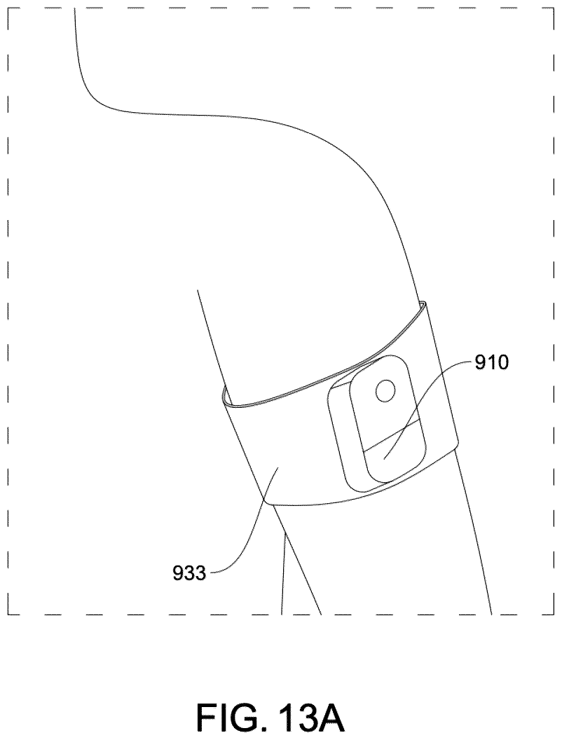

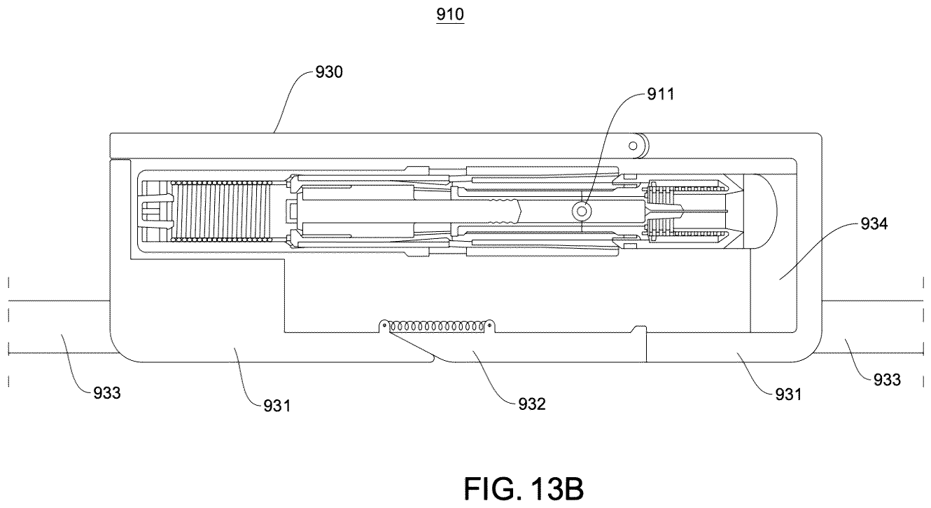

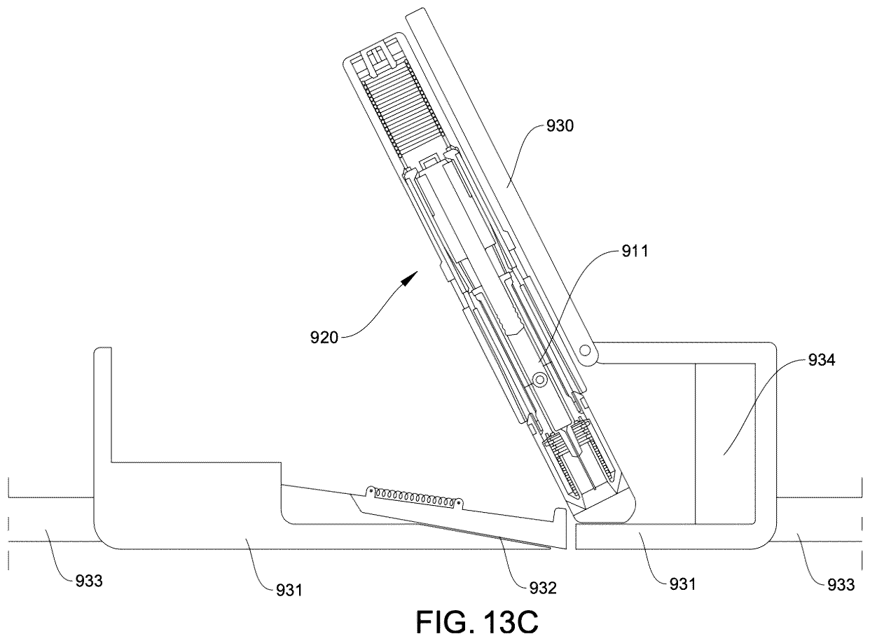

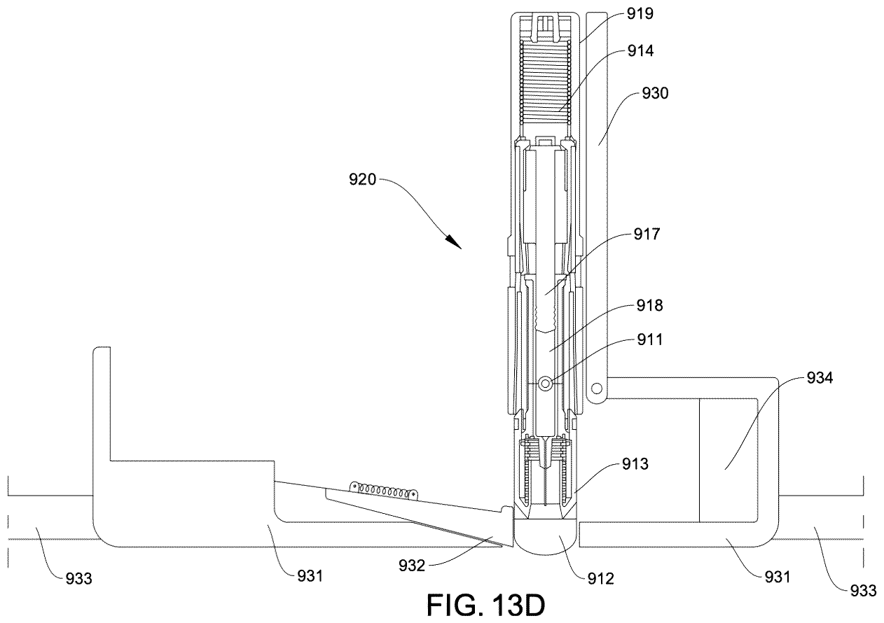

FIGS. 13A-13D depict an example of a parenteral medication administerer 910. In this example, the parenteral medication administerer 910 is configured as an arm band 933 to be placed over the deltoid of a patient. However, in another example, the parental medication administerer 910 can be modified to deliver the medication to a different body location such as the abdomen with a patch for a subcutaneous injection, or on the thigh (vastus lateralis) or buttock (ventrogluteal muscle) for an intramuscular injection. Further, the band shape may be modified to prevent tempering by the patient. Perseris is a subcutaneous injection of risperidone that can be administered every 4 weeks. Such a subcutaneous injection can be delivered to the subcutaneous fat of an abdominal area. Thus, the parenteral medication administer can be configured as an electronic device mounted on a patch and placed on the abdomen.

In FIG. 13A, an example of a parenteral medication administerer 910 is removably mounted over the deltoid of a user by an arm band 933. The parenteral medication administerer 910 may include a housing 931 with a lid 930, a sliding door 932 formed in the housing 931 for passing an injection needle, an autoinjector 920 disposed inside the housing 931 so as to pivot around an axis 911 as to dispose the needle 913 perpendicular to the injection surface. The autoinjector 920 can be placed horizontally in the housing 931 when the patient does not need an injection. By placing it horizontally and parallel to the surface of the body, an accidental injection can be prevented. In this example, the device 10 may alert the user that he/she needs an injection. The schedule of the injection and the dose would have been preapproved by a clinician, or it may be an emergency medication preapproved by a clinician to timely respond to a specific situation of the user. If the user agrees to receive the injection, the user can manually open the lid 930 while wearing the parental medication administerer 910 on his/her deltoid with the use of the armband 933. As the autoinjector 920 pivots around its axis 911 to a vertical position as the lid 930 opens as illustrated in FIG. 13D, and the lower tip of the autoinjector 920 abuts an edge or a protrusion of the sliding door 932 to slide it open. Further, the autoinjector 920 can be provided with an antiseptic pad 912 provided at the bottom tip of the autoinjector 920. As the autoinjector 920 pivots to a vertical position, the antiseptic pad 912 is configured to wipe the skin surface of deltoid muscle that is now exposed through the opening provided by the sliding door 932. Thus, when the autoinjector 920 arrives its vertical position, the injection site is sterilized by the antiseptic pad 912. The antiseptic pad 912 may include ethanol, isopropyl alcohol, or other antiseptic fluid soaked into a cotton pad or be provided as a hydrogel, silicon or sponge that comes in contact with antiseptic. The antiseptic pad 912 may comprise of a gel that contains an antiseptic such as isopropyl alcohol or ethanol to wipe the surface of the skin before actuating needle to deliver the medication. Alternatively, the antiseptic pad 912 may comprise of a cotton pad, fiber pad, sponge or the like that is soaked with an antiseptic fluid. The antiseptic pad support 934 may be made of a sponge or gel and may also include antiseptic fluid. In another embodiment, the antiseptic fluid may be stored in the antiseptic pad support 934, such that the movement of the antiseptic pad as the autoinjector pivots around the axis 911 causes the pad to become wet with the antiseptic fluid. In either events, the antiseptic pad can achieve sterilization of the skin injection site before the needle is ejected for the injection.

In another embodiment, an antiseptic pad 912 can be provided at the bottom of the sliding door 932, and the pushing of the notch of the sliding door 932 can squeeze an antiseptic holding sponge or hydrogel, thereby releasing antiseptic to the area wiped by the sliding door 932. Thus, the injection site can be sterilized before an injection.

Further, in yet another embodiment, the autoinjector may be designed to avoid tempering. In an acute inpatient psychiatric hospital, measures must have to be taken to avoid patients from getting hold of any sharp object that may be used against themselves or the hospital staff. The parenteral medication administerer 910 may be configured to take vitals via a biosensor and to transmit the vital signals via a wireless connection; thus, the clinician will be readily able to determine whether the patient has taken off the device and possibly misusing the device. The surface of the device can be covered with a locking mechanism, and a smooth and soft surface may be provided to prevent injury.

The antiseptic pad 912 may be made of a gel. Examples of suitable materials include: a hydrogel, collagen hydrogel, silicon pad with a textured surface; an antiseptic fluid may be transferred from the support which may include alcohol gel or other antiseptic, like an automatic stamp mechanism. Thus, a small amount of antiseptic can be transferred to the skin each time.

In another embodiment, antiseptic liquid may be released upon opening of the sliding door 932, and may squeeze the antiseptic onto a pad provided below the sliding door. Causing the injection surface to be cleaned. Very minimal sensitivity is envisioned as the temperature of the fluid will be equal to body temperature. For fluid, an ethanol gel may be disposed. The movement of the sliding door 932 can help a weeping movement to take place on the skin, and a dry pad can dry it. Then IM injection can be delivered.

A hydrogel is a cross-linked hydrophilic polymer network. A hydrogel does not dissolve in an aqueous environment, but can soak in a great amount of the hydrophilic fluid.

The autoinjector 920 may include a medication chamber 915, piston 917, springs 914 other mechanism for needle and medication ejection, needle 913, and/or actuator 919. The needle 913 can be a fine needle suitable for subcutaneous injection or a slightly bigger needle for intramuscular injection. Various embodiments of autoinjector can be adopted. Refer to U.S. Pat. No. 8,647,299 for an example of an autoinjector that can be incorporated into the parental medication administerer 910. While a cylindrical autoinjector 920 is illustrated, the autoinjector and its chambers can assume many other geometric shapes.

According to one example, the autoinjector 920 can be triggered to release the needle and the medication by pushing its actuator 919 manually. However, automatic injection is also possible by utilizing appropriate electronic components and wireless communication port, such as a Bluetooth port that communicates with the intervention manager 800. Further, the parenteral medication administerer 910 can be configured to open automatically and to pivot the autoinjector 920 to the appropriate position for delivering the injection.

Further, according to another embodiment, an electronic autoinjector can be provided, and the needle can be actuated by an electronic signal without pushing the actuator 919. The administerer 910 provides an antiseptic pad 912 that automatically wipe the surface of the skin before injection. Thus, even a person without medical training can successfully receive a parental injection without the direct involvement of a healthcare professional. The syringe mechanism may be configured to discharge a predetermined amount of medication. The medication container may be connected to a sealable opening through which medication can be loaded by use of a sterile syringe. For example, the piston may be made of rubber, and a needle can be used to load the autoinjector 920. In another example, the needle 913 can be used as a passage to load medication into the medication chamber 915 by a healthcare professional. At the time, a specific amount of medication may have been ordered and loaded. The user can wear the band 933 when necessary to receive a predetermined amount of medication in the absence of a healthcare provider. A physical button 919 can be provided on the parenteral medication administerer 910 to trigger the injector and dispense the medication.

According to one embodiment, the parenteral medication administerer 910 may include a processor, memory, Bluetooth port, and may include a chamber 915 for storing a psychoactive medication, a needle 913 that can automatically inject a predetermined amount of the psychoactive medication, and a trigger mechanism that inject the needle 913 into the body and release the appropriate amount of medication when placed on skin surface. The parenteral medication administerer 910 can be made into a wearable device, such as a band around the deltoid or made into a peripheral device that can be placed on appropriate injection administration surface by the user.

The parenteral medication administerer 910 may store a predetermined amount of medication, and dispense the same automatically based on a setting selected by the physician in advance. According to one embodiment, the parenteral medication administerer 910 may store injectable antipsychotic medication. In response to the patient endorsing the presence of auditory hallucination or other psychosis, the intervention manager 800 can initiate the Rx intervention initiator. The Rx intervention initiator can inquire the user whether the user would like to receive a predetermined amount of antipsychotic pre-approved by the psychiatrist in advance, in anticipation of the event that the patient may feel he/she needs the additional antipsychotic medication to better control his/her psychosis prior to the next appointment.

Further, medication compliance is a big issue in administering antipsychotic medications to patients who are suffering from the worst form of psychosis. The parenteral medication administerer 910 can be configured to deliver medication automatically weekly, monthly, biweekly, every 6 weeks, and the like, as recommended by the FDA approval for a parenteral medication manufacturer upon pre-approval of a clinician.

In another embodiment, the parenteral medication administerer 910 may be configured to hold a manufacturer produced syringe. Some commercially available long-acting injectable antipsychotic medications come in a preloaded syringes with a pairing needle. However, most patients go to visit a clinician to regularly receive this medication because they do not know how to administer an intramuscular injection on their own. The parenteral medication administerer 910 may be configured to hold a pre-loaded syringe holder instead of the medication chamber 915 and piston 917, allowing patients to receive injection without coming into the doctor's office. The parenteral medication administerer 910 may be equipped with multiple needle 913 to allow automatic needle switching, thus allowing several injections and eliminating the need to come to a clinician's office for several months.

Injectable antipsychotics that may be dispensed by the parenteral medication administerer 910 includes Abilify (Abilify Aristada, Abilify Maintena), Haldol decanoate, Invega (Invega Trinza, Invega Sustenna), Risperdal Costa, Perseris (subcutaneous Risperidone), Zyprexa Relprew, and the like. It is expected new formulations of parenteral medications will hit the market if view of the parenteral medication administerer 910 provided in this disclosure, which may be incorporated into this application. In fact, the parenteral medication administerer 910 makes it easy to deliver antipsychotic more frequently.

Accordingly, it is possible to deliver a smaller amount of antipsychotic more frequently, thereby avoiding the risk of extra-pyramidal side effect, dystonia or tardive dyskinesia, which are common side effects of having a large amount of antipsychotic administered.

It is noted that the device 10 can determine whether a patient has a history of movement disorder, such as extra-pyramidal side effect, tardive dyskinesia and dystonia, and allow the user and the clinician to take an extra-precaution. For example, the camera of a smart phone which functions as the user terminal 100 may take video of the facial expression of the patient to determine whether there is abnormal stereotypical movement of eyes or lips. The accelerator provided in the user terminal, and EEG are also able to pick up abnormal hand movement, tremor, or eye blinks. EEG electrodes attached to the forehead at night for determining sleep architecture is also able to pick up eye blinking.

According to one embodiment, a full Abnormal Involuntary Movement Scale (AIMS) evaluation can be performed by an AI avatar on a patient who receives a parenteral injection. For example, the device 10 can collect information related to AIMS test at several different time to determine whether it is safe to deliver the medication and/or whether the patient may be developing a side effect. For example, the device 10 can collect information regarding abnormal movement before delivering an injection, within 30 minutes to two-hour period after the injection, for the next few days (two to three days) after the injection, as needed. An AI avatar can ask the patient to show his/her face on the camera of a phone, and ask the patient to even to stick out the tongue, and show the shoulder of the patient, and to hold out the smart phone to determine whether there is any sign of movement disorder, such as dystonia, tardive dyskinesia, or extra-pyramidal side effects. The accelerometer can detect any abnormal movement of the hand or shoulder, and the camera can be used to examine the typical eye blinking, tongue movements, shoulder movements and the like. If there is a concern that patient is developing abnormal movement, the patient can be alerted to the fact, and prompted to speak with his/her psychiatrist or clinician. The device 10 can directly inform the clinician or send a notice to the clinician. The device can also inquire the patient whether he/she wants to be connected to a human clinician over the Internet. The device 10 can also update the subtype to “with possible extra-pyramidal symptom” or “with possible abnormal movement due to antipsychotic side-effect” and alert the intervention manager 800 so that precautions can be exercised before administering additional long-acting injectable medication. Thus, the device 10 makes it possible to monitor patient closely for abnormal movements even when the patient is not able to be directly examined by a clinician. Thus, with the use of this device, the clinician will feel more assured that the patient can receive the much-needed monitoring even when the clinician cannot constantly monitor the patient. Further, the patient can receive a timely intervention even if there are not sufficient social resources to provide a 24-hour monitoring of the patient's symptoms.

The parenteral medication administerer 910 can be configured to also deliver an anticholinergic medication as well as an antipsychotic medication. Some patients with severe psychosis need to take antipsychotic medication even if they have abnormal movement disorder. In such a case, the injectable antipsychotic can be administered in conjunction with an anticholinergic in the dose predetermined by the physician to suppress extra-pyramidal side effect. Further, the device 10 can be used to monitor specific extrapyramidal symptoms such as hand tremor, eye blinking, jaw movements in patients who is already known to have extra-pyramidal side effects. For example, if the patient has the subtype “schizophrenia with psychosis” “with auditory hallucination” “with psychosis responsive to long-acting injectable” “responsive to Invega Sustenna IM 156 mg Q4 weeks via deltoid” “with extra-pyramidal side effect controlled by benztropine”, the parenteral medication administerer 910 can be configured to deliver an IM version of the benztropine or the intervention manager 800 can prompt the patient to take PO benztropine twice per day, or as indicated by the clinician. For a patient with known extra-pyramidal symptoms, an EEG electrode placed on the forehead can pick up eye blinking and many facial movements, while an accelerometer in a smartphone can be used to pick up occasional hand tremor. A portable electromyographic technique or placement of electrodes or sensors on skin or arm can also provide additional information regarding extra-pyramidal symptoms. A camera can also pick up eye-blinking or tongue movement. The AI avatar can ask the patient to open the mouth and stick out the tongue, so that the camera of the user-terminal can pick up abnormal tremor. An automatic report can be generated for the physician to inform the physician of any abnormal movement or the use of additional antipsychotic medication.

According to one example, a fully automated parenteral medication administerer 910 can be configured to deliver medications regularly to a patient who has poorly controlled psychosis. For example, the parenteral medication administerer 910 can be mounted on a patient who lives in a group home or an inpatient unit or who has suffered an acute first episode of psychosis. The automated parenteral medication administerer 910 may be configured to deliver the medication directly to the patient based on physician authorization. For instance, if the patient is agitated and code strong is called, and the patient cannot be calmed down through verbal de-escalation, it may be necessary to deliver an extra Haldol injection to the patient. Currently, the psychiatrist usually ends up calling the hospital security guard and MedTech to hold down the patient safely while a nurse quickly advances towards the patient to deliver an IM medication such as IM B52 (Benadryl, Haloperidol 5 mg, Lorazepam 2 mg). This kind of operation requires a lot of manpower, as well as exposing the hospital staff to an extreme risk to injury and emotional stress. With the present device, a patient can be provided with a parenteral medication administerer 910 upon admission to the hospital, and the physician can order the administration of the emergency medication via the healthcare provider terminal 120 when verbal escalation fails to calm the patient. Thus, the nursing staff and the MedTech can perform their job more safely and with less emotional trauma with the assurance that the parenteral medication can be delivered without physically tackling an agitated patient.

According to one example, the autoinjector 920 can be configured to that it does not have to pivot to a vertical position. The chamber and the needle can be changed in shape so that a small device can be provided while delivering the injection without autoinjector repositioning. This design would be particularly useful in an inpatient unit with acutely ill patients for whom a quick administration and frequent administration of the injectable medications become important.

The parenteral medication administerer 920 can be configured with more than one autoinjector 920. For example, the parenteral medication administerer 920 can be loaded with two autoinjectors 920, one for antipsychotic medication and another for anticholinergic medication. For example, Invega Sustenna may require one injection every four weeks. The patient may need benztropine or other anticholinergic every 8 hours, 12 hours or daily. By having several autoinjectors 920, different medication can be dispensed with different frequency. The parenteral medication administerer 920 can be configured to be loaded with three autoinjectors 920, one for antipsychotic medication, one for anticholinergic medication, and one for benzodiazepine. Alternatively, a mixed emergency medication cocktail, such as B52, can be preloaded to one of the autoinjectors 920 for a rapid administerer. In addition to autoinjection, the autoinjector 920 can house a needle and a chamber for taking a blood sample. The autoinjector 920 can hold the blood sample for a clinician, or have a sensor mechanism for detecting certain key chemicals. For example, the autoinjector 920 can be equipped with a glucose monitoring sensor, a lipid panel monitoring mechanism, agranular cytosis monitoring or complete blood count (CBC), or a liver enzyme checking mechanism. This information is important for ensuring proper metabolic status of a patient taking a large amount of antipsychotic medication. The autoinjector 920 or other wearable device such as a wrist band can provide information regarding body fat percentage, body water content, blood pressure, heart rate, EKG, and the like. Some peripheral devices such as a digital weight can provide the weight of the patient, and BMI can be calculated from the weight and height of the patient. This information can be provided to the assessment unit 300 to determine whether the patient is “with metabolic syndrome” or “with obesity” and the like. This information can further be further used to determine which antipsychotic is more appropriate for the patient. For example, the device can alert the psychiatrist that a patient's body weight has rapidly increased after being placed on an antipsychotic, and that the patient may benefit from a different antipsychotic. The device 10 can provide a list of medication that are less like to cause metabolic syndrome on its physician report generated by report generator 900.

Several embodiments of the device 10 for dynamically determining the subtype of a psychiatric disorder and providing personalized medical care based on the dynamically determined subtype were described above.

The user interface unit 100, 120, 130 can provide patient informing mechanisms, family or social support informing mechanism, and a healthcare provider informing mechanism. The healthcare provider terminal 120 can also provide the healthcare provider with the option of determining a predetermined setting for the device. For example, the healthcare provider can control which intervention will be offered by the intervention manager 800, and set the amount of medication that is to be dispensed by the parenteral medication administerer 910. Thus, it is possible to provide a continuous care in between the appointments for physicians, and automatically keep track of the disease progression of the patient in the absence of the physician.

The user terminal 100 can also provide the patient with an option of connecting with an online therapist, an online psychiatrist, or a suicide hotline. Thus, a patient can be assured that additional clinical support can be easily obtained without going into a hospital.

Further, the healthcare provider terminal 120 can provide a clinician with a remote way of connecting with a patient who may be experiencing a side effect, such as abnormal movement, after receiving an antipsychotic medication. The physician can also load an emergency antipsychotic or other medication on a parenteral medication administerer 910 to allow the user to take extra dose as needed. A physician working in the emergency room or in an involuntary psychiatric hospital can use the healthcare provider terminal 120 to order the administration of an emergency medication via a parenteral medication administerer 910 without having to engage in an altercation with an agitated patient to administer the injection to the patient's buttock or deltoid, reducing the risk of injury for MedTech and other hospital staff. The healthcare provider terminal 120 also provides the clinician with an opportunity to monitor the subject mood of a depressed patient or the manic symptoms of a bipolar patient without having to bring the patient into the clinic. Accordingly, patients can be better managed even in rural areas where there is a physician shortage.

Further, the device 10 makes it possible to dynamically update the patient's subtype based on new information that are continually gathered regarding the patient's disease, its responsiveness to intervention, the patient's subjective experience of the disease, and the evolving progression of the disease. The device 10 gathers biometric data, phenotype data, endophenotype data, subjective mood input, genetic information, structural and functional differences in the brain, activity level, physical location of the movement, side-effects, list of efficacious interventions for the specific patient and the like into one dynamic data structure. Thus, it is possible to provide dynamic and personalized medical care to the patient.

The hardware of the device 10 and subunits of the device may include a central processing unit, electric circuits, Bluetooth, or other wireless communication device, wired communication, biosensors, peripheral device ports and the like. The biosensors may be embodied on a smart watch or other wearable device, a mobile device such as a smart phone carried by the patient, or a peripheral sensor that communicates with a primary processing device such as a BIS spectra strip placed on the head of the patient that communicates with a smart watch, a smart phone, or a computer via wireless communication, wired communication, Bluetooth, or by intermittent manual download.

According to one embodiment, an algorithm for subtype determination may include the steps of: (1) determining the DSM criteria of a disorder, such as whether the disorder appears to be a mood disorder or a psychotic disorder or a personality disorder or others. (2) If mood disorder, determining whether the disorder appears to be a Bipolar I disorder involving mania, Bipolar II disorder involving hypomania, cyclothymia, unipolar depression (no hypomania or mania), schizoaffective disorder. If it is a psychotic disorder, determining whether it is a schizoaffective disorder, schizophrenia, schizophreniform disorder, brief psychotic episode. If it is anxiety disorder, determining whether it is a panic attack, a generalized anxiety disorder, a phobia, OCD, and the like. The initial diagnosis can be obtained from the patient's initial data collection, based on prior diagnosis, based on a working diagnosis provided by a healthcare professional, or based on scores obtained from various questionnaires or psychological batteries, such as PHQ9, Hamiltonian Anxiety Scale, and the like. The diagnosis can be taken from inputs from family members or the subjective opinion of the patient. There can be conflicting diagnoses. The assessment unit 300 may be provided with a hierarchy of evidence, and a point system can be applied to each diagnosis based on the strength of evidence. For example, a clinical diagnosis by a current licensed physician would be considered with a greater weight than the results of reports obtained from completed questionnaires, such as PHQ9 and Hamiltonian Anxiety Scale. If there is no clinical diagnosis available, the score report obtained on a questionnaire like PHQ9 becomes important and takes precedence over the subjective opinion of the patient or a family member.

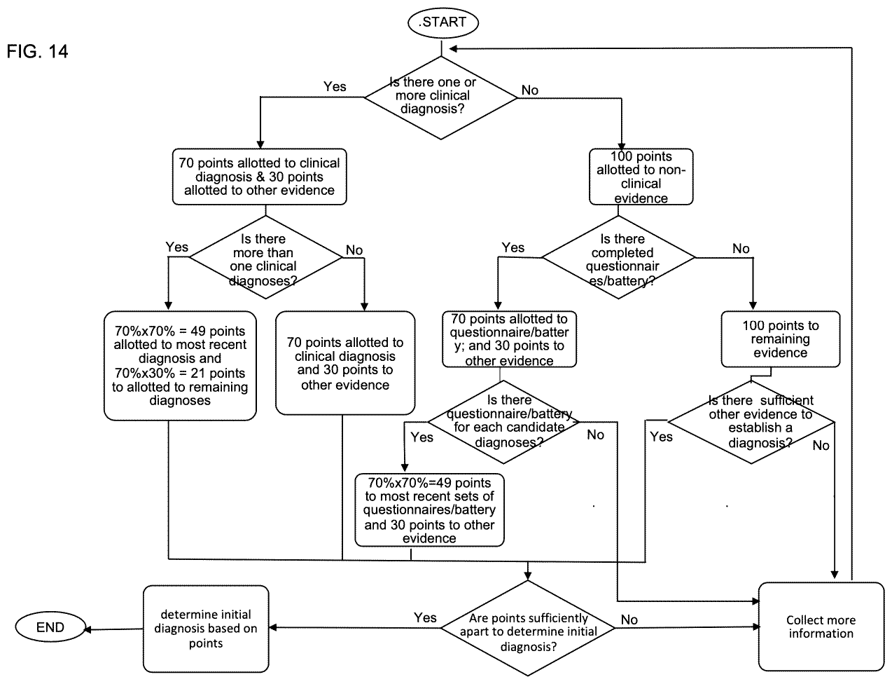

FIG. 14 depicts an example of a method of determining the initial subtype of a patient. The device 10 needs to determine an initial subtype of a new patient. Thereafter, the subtype can be updated as new information is gathered.

According to an embodiment, the determination of initial subtype is made based on a hierarchy of evidence and a point distribution system among candidate diagnoses. For example, the initial data collection unit can collect as much initial data as available or provided by the patient, clinician, and family members. However, often the patient may be a poor historian or might not understand what is going on. The processor of the assessment unit 300 can determine whether the user ever received a clinical diagnosis for his/her condition from a clinician. If the user received one or more clinical diagnosis, then, a distributed weight or 70% of the points may be assigned to the one or more clinical diagnosis. That is, in determining the initial diagnosis of a patient, the device 10 values clinical diagnoses above other forms of evidence. The distributed weight can be set to different numbers in different examples. For example, in another embodiment, 80% of the points can be assigned instead of 70%.

If there is one prior clinical diagnosis of bipolar I disorder, and answers to the patient's completed questionnaire points to major depressive disorder, 70 points out of 100 will be assigned to the candidate diagnosis of bipolar I disorder and 30 points out of 100 will be assigned to the candidate diagnosis of major depression. Since 70 points is greater than 30 points, the initial working diagnosis will be bipolar I disorder. This initial working diagnosis will be stored as the current subtype of the disease in the assessment storage 700 until the current subtype can be updated based on additional evidence.

If there are two or more clinical diagnoses, the device 10 gives more weight to a recent clinical diagnosis rather than an old diagnosis. For example, if there are two diagnoses and answers to a completed questionnaire, with the first diagnosis being bipolar I disorder assigned by a physician in 2020, and the second diagnosis being major depression from 2010 by a nurse practitioner, the candidate diagnosis of bipolar I disorder from 2020 receives 70% x70%=49 points out of 100, and the candidate diagnosis of major depression receives 70% x30%=21 points. The result of current questionnaire points to major depression rather than bipolar I disorder, which receives 30%=30 points. Combining the results, the candidate diagnosis of major depression receives 51 total points, making major depression the most likely candidate subtype. At this time, the initial subtype can be determined to be major depressive disorder. However, given that 49 points and 51 points are very close to each other, the assessment unit 300 may determine that “points are not sufficiently apart” to determine the initial diagnosis. That is, the device 10 may require the first candidate diagnosis to have 50 or more points, or more probably than not, to accept it as the initial diagnosis. If a greater certainty is required, the first candidate diagnosis may have to have at least 75% of the points, for example. In the event that the points are not sufficient apart to ensure the candidate diagnosis is correct, the data collection unit 200 can be activated to collect more information that is relevant to clarifying the initial working diagnosis. For example, the data collection unit 200 may collect additional data regarding symptoms of major depression and bipolar disorder, such as a change in sleep pattern, excessive guilt, a history of having a time period of 7 days or more in which the user experienced pressured speech, grandiosity and/or increased activity, and the like. The assessment unit can use the difference in diagnosis criteria provided in DSM-5 among the candidate diagnoses to come up with its tailored question set, thereby reducing the workload on the clinicians and the patient. As shown in FIG. 14, with the receipt of additional information, the process of determining the initial subtype can be repeated.

If a prior clinical diagnosis does not exist or cannot be accessed by the device 10, the initial working diagnosis can be determined solely based on symptoms provided by the user and other information such as biometric data obtained from user via wearable or portable electronic devices.

In this setting, a series of questions can be asked to determine the most likely diagnosis. For example, the data collection unit 200 and assessment unit 300 can gather specific information to determine whether there is a potential mood disorder, such as dysthymia, hyperthymia, euthymia, subjective mood input, grandiosity, irritability, anxiety, excessive guilt or the like. Questionnaires such as PHQ9 can be completed by the patient with the aid of an avatar. An accelerometer, speech recognition device, biosensor and the like can determine the presence of akathisia, pacing around, pressured speech, increased rate of speech, blood glucose, heart rate, heart rhythm and other relevant information to determine hypomanic state, manic state, or depression. If the condition is believed to be a mood disorder, the presence of psychosis can be determined with a qualifier such as “with psychosis.” More specific qualifier of psychosis, such as “with delusion”, “with paranoia”, “with ideas of reference”, “with auditory hallucination”, “with tactile hallucination”, and the like can be explored based on other available evidence or by using an avatar to ask questions to the patient. If there was an episode of mania or psychosis, the data collection unit 200 may collect the best available data regarding the year when these episodes happened, as well as the duration of the active episode.

Clinical history such as number of prior episodes, recurrence of disease, prior hospitalization, frequency of rapid cycling, prior severity (hospitalization=severe, recurrent=more than 2 times, rapid cycling 4 or more manic episode in 1 year) can further define the subtype. Prior history of suicide attempt, suicide ideation, homicide ideation can further define subtype. Some of these information can be input by the clinician by looking through prior medical history of the patient.

Substance abuse, hallucination or delusion elicited by a medication or substance, increased substance use during manic episodes and the like can further define the subtype. It may be necessary to obtain a toxicology panel. The blood sample can be drawn by the parenteral medication administerer 910.

Structural or biological abnormalities, abnormal shape or size of the brain, abnormal neural network connections as determined by fMRI, hypoactivity or reduced function of certain parts of the brain, such as abnormal corpus callosum function, white matter abnormality, reduced gray matter, global brain atrophy, traumatic brain injury, history of stroke can further define the subtype.

Brain wave-based data, normal or abnormal EEG, normal or abnormal sleep architecture, detected seizure, psychogenic nonepileptic seizures (PNES), and the like can further define the subtype.

Genetic information, endogenetic information, prior family history can also further define the subtype.

Phenotypic delineation such as sleep pattern, decreased need for sleep at the onset of a manic episode, seasonal affective disorder, diurnal mood fluctuation can further define subtypes.

Behavioral symptoms such as money spending issues during manic episode, history of engaging in risky or reckless behavior during manic episode, extremely grandiosity during manic state, extreme irritability or anger management issue during manic episodes, prior conflict with law enforcement during manic state or psychotic state, prior suicide attempt or self-injurious behavior during depressive episode and the like can further define the subtype. By storing such information in the subtype, the device 10 can dynamically provide the user with a tailored message to avoid making important decisions during a manic episode, to increase exercise at the onset of depression, to contact a favorite social support, to call the suicide hotline, and the like.

Intervention responsiveness and pharmacological responsiveness can further define the subtype. For example, a major depressive disorder can be sub-classified as “responsive to sertraline” but “not responsive to Wellbutrin.” ECT and TMS responsiveness can also define a subtype. For those with psychosis, responsiveness to a specific antipsychotic such as PO olanzapine can further define a subtype. Such a patient can be offered an extra dose of olanzapine during a psychotic episode with the prior approval of the psychiatrist.

The initial subtype classification will be continually checked against new information, such that the subtype classification is dynamically updated. For example, monitoring of mood and other symptoms over time may show a subtype change to a rapid cycling due to increased frequency of manic episodes to four or more per 12 months period. Patient may be diagnosed with seasonal depression due to the development of depressive mood during a winter season that improves in the spring.

By using a tree nodal data structure to define the subtype, it becomes possible to detect or even predict a new onset of manic episode or depressive episode. For example, in November, a patient with seasonal affect disorder can be counseled to start a light lamp therapy even before experiencing deep depression based on the patient's own mood inputs from the last three years. Patients can be informed regarding the onset of symptoms so that preventative steps can be taken. In another example, a patient who is starting a manic episode can be reminded to sleep more or take extra sleeping aid or make an appointment with a physician to adjust pharmaceutical regimen.

Pressured speech picked up by the microphone or a psychomotor agitation sensed by an accelerometer can be used to alert the patient of a potential new onset of manic episode. If the patient has a bipolar disorder “with history of taking on risky behavior during manic episode”, the patient can be advised to avoid making important decisions until further treatment. If the condition is characterized by going on travels during manic episode, the patient's whereabout may be revealed to the social support. Alerting the parents would be especially important for a minor or a patient with severe psychosis that can cloud the patient's insight and judgment.

Physicians can be alerted regarding new hospitalization, medication changes, use of an emergency antipsychotic medication, worsening symptoms of psychosis, suicide ideation, side effects, and the like. The physician can intermittently interrogate the device, download reports generated by the report generator 800, or opt to receive notice for certain events.

The device 10 can provide continuous personalized medical care between physician's appointments. The physician can pre-select algorithms for dealing with potential clinical issues that the patient may experience prior to the next appointment. For example, continued sleep deprivation resulting from decreased need to sleep can result in a full-blown manic episode. In response to detecting a change in sleep pattern, the intervention manager 800 can instruct the patient to initiate melatonin or other sleep aid chosen by the psychiatrist in advance. The patient may have had a history of becoming irritable during manic episodes, resulting in relationship issues. The intervention manager 800 can deliver therapeutic messages to calm the patient down; provide music therapy or digital dialectic behavioral therapy (DBT) therapy or reflective network therapy; provide inspirational or spiritual messages; or get the patient connected to an online human therapist.

According to one embodiment, the intervention manager 800 can provide a virtual mindfulness exercise. For example, an AI avatar may confirm from the patient that he is feeling depressed and anxious. In response to detecting a depressive mood based on the user input, the intervention manager 800 can utilize an AI avatar to ask, “Can I offer you your favorite music with my biofeedback monitoring?” If the patient answers yes, a piece of music can be offered via a speaker while biosensors are used to determine the patient's heart rate and the like.

According to one embodiment, a virtual therapy can be offered by an AI character. For example, the virtual avatar can say, “You are telling me that you don't feel so great. Do you want to have a short talk session with your virtual friend, Pet Charlie, now? How about 5 minutes?” “Are you endorsing that you have suicidal thoughts?” “I am concerned about you, because you are a pretty awesome human being. Can I connect you to a suicide hotline or connect you to your social support: Aunt Jenny?” The intervention manager 800 can also offer getting extra exercise, by stating, “when you felt depressed in the past, your mood improved after swimming. How about going for swimming sometime today?” “You appear to be suffering from seasonal affective disorder. Can I help you get light therapy by turning on your light therapy lamp in the morning?”

An automatic medication dispensing may be performed via the intervention manager 800. The intervention manager 800 may be designed to dispense medication via a parenteral medication administerer 910. The intervention manager 800 may offer neuro-modulation treatment in some patients. A variety of interventions may be offered to the user, including but not limited to anti-depressant medications, anti-psychotic medications, TMS, neuromodulation therapy, virtual therapist, virtual DBT session, connection with an online human psychiatrist, connection with an online human therapist, and the like.

Bipolar disorder is not one disease. While DSM V categorizes the spectrum of affect disorders by grouping them based on their phenotypes, the etiology of each affect disorders, their phenotypes, disease progression and effective treatment are different for each patient. There may be multiple reasons that a patient developed the symptoms of and became diagnosed with bipolar I or II disorder or depression. The etiology may include varying degrees of genetic contributions, childhood exposure to trauma that may have affected brain molecular or network structure due to brain plasticity, organic causes such as traumatic brain injury, stroke, ischemic events, exposure to psychological trauma in childhood, exposure to chemicals in environments, substance abuse, as well as psychosocial developmental issues such as childhood neglect, abuse, pathologic emotional conflict with siblings or caretaker, relationship issues with opposite sex, and personality formation such as ego and superego development.

Bipolar disorders can also be categorized based on treatments that works for a particular patient. Some patients will respond to light therapy for their depression, especially if they have comorbid seasonal affective disorder. Others may respond mostly to medication management such as the use of Lithium, Anticonvulsant (Depakote, Lamotrigine) or second generation anti-psychotic medications (Abilify, Seroquel). The medication that used to work for a patient may no longer work in the future or the patient may develop a side effect, such as tardive dyskinesia or metabolic syndrome, that causes the patient to discontinue the medication. In that case, the patient will have to seek out another medication or treatment modality. Some patients may respond well to therapies, such as dialectic behavioral therapy, cognitive behavioral therapy, or psychodynamic therapy. Others may benefit from mindfulness and meditation, listening to music or connecting with spirituality. Other patients may need a timely coaching based on the onset of their manic episode. For example, they may benefit from a device that reminds them that they may be experiencing a manic episode so that they can avoid making rash decisions, making big purchases, or engaging in new projects which they may later regret in the long term. Many bipolar disorder patients also experience extended time periods of depressive mood. The device can benefit patients by helping them realize that they may be experiencing a depressive episode, so that they can avoid making harmful decisions such as suicide attempt, allowing their work-performance to suffer, or causing relationship mistakes with significant others. The device can help the patient find activities that help them cope during their depressive episodes, such as getting out of bed and exercising. The device can further track interventions that may have worked in the past, such as listening to music, making an appointment with a therapist or a psychiatrist, seeking out social support, trying out light therapy, increasing exercise and healthy eating, and suggest or provide the specific intervention to the patient in a timely manner.

Continuous monitoring of the biometrics of a bipolar disorder patient can be performed with a wearable or portable device, such as a watch, a phone, a head band, an electrode, a strip attached to the body, patches, cufflinks, or tattoos with sensors. These biometric data include valuable information which provides a glimpse into the mental state of a patient. Inventor has determined that, by collecting the biometric data over an extended time, such as 1 month, 6 months, or 1 year or more, the biometric data can be used in an algorithm to provide a bird eye-view insight into characterizing the patient's disease, and the additional qualifiers of subtype can be used to devise a short-term treatment plan for the specific patient in between his/her appointment with psychiatrist. In other word, a continuous care can be provided automatically without the continuous involvement of psychiatrist according to an algorithm preset by the psychiatrist or preset by the device. Further, the device can deliver appropriate medicine and other therapeutic to patients with and without the conscious involvement of patient or doctor.

Continuous monitoring may allow the determination of the specific subtype of the psychiatric disorder a particular patient has, the type of treatments to which the specific patient responds best, and the symptoms that the particular patient is likely to experience at the onset of a manic episode or depressive episode, and to monitor a change in these patterns of symptoms specific to the patient by updating the subtype of the disorder that a patient has. In other words, it is possible for this device to provide a truly personalized medical care to the patient by monitoring the phenotype, treatment efficacy and the like that are specific to the patient over an extended amount of time, in a manner that it would be difficult for a human psychiatrist to perform due to the limited time that a human psychiatrist can spend on one patient, due to the inability of the human psychiatrist to stay with the patient at all times or measure his vitals and other biometric data, and due to the fact that the human psychiatrist who takes care of a patient keeps changing during the lifespan of the patient due to the reality of lack of continuity of care, due to the change in circumstances of the patient, and the wide range of medical institutions, inpatient, outpatient and partial services, provided by the society to take care of the disease based on varying degrees of severity.

Provided herein is also a method for monitoring and managing a neuropsychiatric condition with the use of biosensors. The method involves steps of: data collection, running algorithm for subtype determination; dynamically detecting a change in subtype overtime based on new information to determine a detailed subtype of the disease that is specific to the patient; informing patient of clinically relevant findings based on processing the data collected by biosensors; informing family members of the clinically relevant findings; informing physicians of the clinically relevant findings; providing a remote continuous and personalized care to the patient in a dynamic manner based on the continuous receipt of new information; and automatically dispensing medications or providing intervention based on dynamically received new information.

The hardware of the device that performs the monitoring and managing of the psychiatric disorder may include: a central processing unit, electric circuits, Bluetooth or other wireless communication device, and biosensors. The biosensors may be embodied on a smart watch or other wearable device, a mobile device such as a smart phone carried by the patient, or a peripheral sensor that communicates with a primary processing device such as a BIS strip placed on the head of the patient that communicates with a smart watch, a smart phone, or a computer via wireless communication, wired communication, Bluetooth, or by intermittent manual download.

The data collection step may involve collecting information regarding: sleep pattern, movement via accelerator; physical location and travels via GPS; online shopping or online transaction; brain wave information via EEG electrodes or BIS strip; QTc via EKG electrodes; facial expression and speech change via camera and microphone; vitals such as heart rate, pulse pressure, temperature, fever status; sweat level; akathisia, tremor, abnormal movements; answers to questionnaires to determine severity; daily subjective mood; lithium level; Depakote level; Lamictal level; urine toxicology and substance use data, such as the use of Cocaine, PCP, LSD, marijuana, alcohol; and the like. A time stamp may be associated with each of the data, so that change can be detected.

An algorithm may be run on a processor to determine the subtype in a dynamic manner. The subtype includes a clinical diagnosis, such as a DSM diagnosis, such as bipolar I disorder, bipolar II disorder, schizophrenia, major depressive disorder, and the like. The presence of psychosis may be checked, as well as the number of past episodes, any history of hospitalization, the frequency of cycling of manic episodes, a history of prior attempted suicide, structural findings of brain such as corpus callosum function and neuroimaging findings, prior substance use, and endophenotype, phenotype, genetic and behaviors issues such as: sleep pattern change, seasonal fluctuation in mood, excessive money spending during an active episode, engagement in risk business during an active episode, engagement in a reckless activity during an episode, judgement fluctuation such as irritability, anger management issues, problems with law enforcement, Illegal activity, suicidal attempt or cutting or self-injurious behavior, substance use, coping skills and coping patterns (what does this person do as coping mechanism and what works).

As an initial data collection, the method may involve determining whether the patient is having the first episode or a recurrent episode, the involvement of any psychosis, 12-lead EKG and QTc, an EEG to determine brain wave, genetic information and/or family history of similar psychiatric diseases, the number of prior cycles, the length of each cycles, the severity of the condition, and possible suicidal thoughts or intent or attempt. Continuous monitoring of biometric data and subject mood is utilized to update the subtype over time.

The method may involve informing the patient regarding his/her current condition. The device 10 can generate an alert to the patient of the risk of new manic episode or psychotic episode, for example. If the patient is a bipolar patient and having a reduced sleep requirement, the device 10 may intervene by reminding the patient to get more, take a sleeping aid preapproved by the physician, or contact the physician.

The method may further involve informing the physician regarding the patient's up-to-date symptoms, a high-level overview of the patient's subjective mood input in a graphical rendering over an extended period of time, for example, one month to several years. The method may further involve alerting the physician for management of the situation, medication change or hospitalization of the patient. The patient may be able to download information from the device or interrogate the device.

The method may involve providing a continuous care between doctor's appointments. The physician can select an algorithm for dealing with minor changes in the patient's life. For example, change of sleep pattern, initiation of melatonin or sleep medication before next appointment can be pre-authorized by the physician outside of an actual appointment with the physician.

The method may involve an automatic medication dispensing or medical intervention. The method may involve providing an automated injection, TMS treatment, ECT treatment, neuromodulation, neurofeedback sessions, and the like.

Also provided herein is a device that can assist patients and physicians during an inpatient admission of the patient. For example, the inpatient admission may have been prompted by a first episode of psychosis in the patient's life.

Such a device can help both the patient and clinician in the ER or at a psychiatric hospital. According to one embodiment, an algorithm-based guidance can be provided to an ER doctor, a primary care facility, or in a small hospital in a remote area without a psychiatrist. The device can walk the clinician through the algorithm for diagnosing a psychiatric disorder, for example by asking relevant questions regarding mood and symptoms to the patient and collecting appropriate questionnaires such as PHQ-9.

The device may further guide the clinician to collect appropriate data and to analyze the situation better by informing the clinician what orders should be placed. For example, the device can prompt the physician to obtain an MRI, EEG, a thyroid and other markers, B12 and glucose. Substance use screening test can be offered to the patient. The device may collect relevant information such as the age of the patient, family history, genetic information, baseline function (normal development, no disease, comorbid disease, autism, depression, mood disorder, psychosis) and the like.

Placement of a hospital band with a wearable sensor, smart watch, head band, hair pin, EKG, EEG, and an autoinjector can make patient care safer in the event of patient agitation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip, a circuit, a processor, a microprocessor, a touch screen, and the like. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

What is claimed is:

1. A device for dynamically determining medical subtype and providing intervention based on the subtype, the device comprising:
a user terminal comprising a processor and memory, wherein the user terminal is configured to receive user input;
a data collection unit configured to collect biometric data from a wearable device and store the collected biometric data in a data collection storage;
an assessment unit configured to process the biometric data to determine presence of a symptom in a user and to determine medical subtype and store the determined medical subtype in an assessment storage,
wherein the assessment unit comprises:
a dynamic subtype update unit that is configured to dynamically update the current medical subtype in response to receiving new biometric data that indicates either the stored subtype is incorrect or needs additional qualifier; and
an alert initiator that is configured to determine timing of offering an intervention to the user; and
an intervention manager is configured to offer the intervention to the user in response to the determining of the alert initiator,
wherein the intervention manager is configured to offer at least one event from the group consisting of patient notification, healthcare provider notification, social support notification, suicide hotline connection, virtual therapy, music therapy, mindfulness therapy, light therapy, RX dispensing, biofeedback session, neurofeedback session, or a combination thereof.

2. The device of claim 1, wherein the medical subtype is a psychiatric disorder subtype, and the assessment unit is configured to process the to process the biometric data to determine an emotional state of a user and to determine the psychiatric disorder subtype and store the determined psychiatric disorder subtype in an assessment storage,
wherein the assessment unit comprises:
a dynamic subtype update unit that is configured to dynamically update the current affect disorder subtype in response to receiving new biometric data that indicates either the stored subtype is incorrect or needs additional qualifier; and
an alert initiator that is configured to determine timing of offering an intervention to the user; and
an intervention manager is configured to offer the intervention to the user in response to the determining of the alert initiator,
wherein the intervention manager is configured to offer at least one event from the group consisting of patient notification, healthcare provider notification, social support notification, suicide hotline connection, virtual therapy, music therapy, mindfulness therapy, light therapy, RX dispensing, biofeedback session, neurofeedback session, or a combination thereof.

3. The device of claim 1, wherein the wearable device is configured to be worn continuously by the user over an intended length of time of one week or longer, and the assessment unit is configured to dynamically update the current subtype based on dynamically received information from the wearable device.

4. The device of claim 1, wherein the wearable device is configured to be worn daily by the user over an intended length of time of one month or longer, and the assessment unit is configured to dynamically update the current subtype based on dynamically received information from the wearable device.

5. The device of claim 1, wherein the wearable device is configured to be worn daily by the user over an intended length of time of one year or longer, and the assessment unit is configured to dynamically update the current subtype based on dynamically received information from the wearable device.

6. The device of claim 1, wherein the intervention manager is triggered to offer an intervention in response to an updating of the current subtype stored in the assessment storage.

7. The device of claim 2, wherein the wearable device comprises an electrode to be mounted on the head of the user to collect brain wave related information,
wherein the brain wave information is used to determine sleep architecture of the user, and wherein a determination is made regarding whether the user is developing a new onset of manic episode by comparing the average sleep length of the user in a range of time between one day to seven days to the average range of sleep of the user in a range of time between a month to twenty years.

8. The device of claim 5, wherein the brain wave information is used to determine sleep architecture of the user, and the sleep architecture is used to determine whether the user has a reduced REM latency.

9. The device of claim 2, wherein the user terminal is configured to collect information regarding the user's daily mood, and the assessment unit is configured to generate a mood chart over an extended time in a range of one week to a hundred years, and the assessment unit determine whether the user is euthymic, hypomanic, manic, or depressed based on the mood chart.

10. The device of claim 8, wherein the mood chart allows the device to determine whether the user is experiencing a rapid cycling of mood.

11. The device of claim 9, wherein the device is configured to alert at least one of the user, a healthcare provider of the user, a social support of the user, or a combination thereof, in response to a determination that the user is experiencing the rapid cycling, and the device is configured to update the subtype to reflect the presence of the rapid cycling.

12. The device of claim 8, wherein the device is configured to alert at least one of the user, a healthcare provider of the user, a social support of the user, or a combination thereof, in response to a determination that the user is experiencing a new onset of manic episode.

13. The device of claim 12, wherein the device alerts the user of the determination that the user is experiencing the new onset of manic episode and warn the user to modify behavior, and the warning comprises at least one selected from the group consisting of getting more sleep, refraining from spending money, refraining from risky behavior, anger management, refraining from big life decisions, stop antidepressant, stop light therapy or modify light therapy, listen to inspirational message, contact social support, contact healthcare professional, take an antipsychotic medication, or a combination thereof.

14. The device of claim 12, further comprising a parenteral medication administerer comprising a psychotropic medication storage, the parenteral medication administerer configured to inject the user with predetermined dose of medication as an intervention in response to a patient consent.

15. The device of claim 7, wherein the mood chart allows the device to determine whether the user has entered a new depressive episode.

16. The device of claim 14, wherein the intervention manager is configured to offer at least one event selected from the group consisting of music therapy, virtual therapy, mindfulness exercise, light therapy, or a combination thereof in response to the determination that the user entered a depressive episode; and a biofeedback related information is obtained via biosensors worn by the user while the event is offered to the user to determine an efficacy of the event on the user.

17. A method for dynamically determining psychotic disorder subtype and providing intervention based on the subtype, the method comprising:
receiving user input from a user via a user terminal comprising a processor and memory;
collecting biometric data of the user via a wearable device;
processing the biometric data on processor to determine psychotic disorder subtype and store the determined psychotic disorder subtype in memory;
continually receiving biometric data from the wearable device over a prolonged length of time exceeding one week, and continually determining whether the psychotic disorder subtype is to be updated; and
dynamically updating the psychotic disorder subtype based on the determination, and initiating an intervention based on the updated psychotic disorder subtype,
wherein the psychotic disorder subtype comprises:
a DSM diagnosis comprising at least one of: bipolar disorder, unipolar depression, schizoaffective disorder, schizophrenia, schizophreniform disorder, adjustment disorder, generalized anxiety disorder, OCD; and
one or more qualifiers comprising at least one of: with psychosis; without psychosis; with single episode; with repeated episodes; with recurrent disease; with rapid cycling; with paranoid delusion; without paranoid delusion; with ideas of reference; without ideas of reference; with hallucination; without hallucination; with auditory hallucination; with visual hallucination; without gustatory hallucination; with history of hospitalization; without history of hospitalization; with history of suicide attempt; without history of suicide attempt; with history of violent behavior; without history of violent behavior; with suicidal ideation; with homicidal ideation; with substance use; induced by substance; induced by another medical condition; induced by neurodegenerative disease; with abnormal neuro structure; with abnormal neuro structure at corpus callosum function; with abnormal white matter structure; with reduced gray matter; with abnormal brain wave; with abnormal sleep architecture; with normal sleep architecture; with reduced sleep at onset of manic phase; with seasonal affective disorder; with diurnal mood swing (AM or PM); with money spending issues during manic state; with risky business decision during manic state; with reckless activity during manic state; with grandiosity during manic state; with depression responsive to neuromodulation; with irritability during manic state; with substance abuse during manic state; with psychosis during manic state; with suicide attempt during depressive state; with psychosis during depressive state; with depression responsive to light therapy; with depression responsive to antidepressant; with depression responsive to mindfulness exercise; with depression responsive to ECT; with depression responsive to TMS; with psychosis responsive to antipsychotic; with genetic predisposition; with family history; with mild symptom; with moderate symptom; with severe symptom; post stroke; post traumatic brain injury; with concern for dementia; responsive to psychotherapy; responsive to antidepressant; responsive to antipsychotic; responsive to long-term injectable antipsychotic; with extra-pyramidal symptom; with history of tardive dyskinesia; with onset of tardive dyskinesia; responsive to anticholinergic agent; responsive to mood stabilizer; responsive to light therapy; responsive to biofeedback meditation; responsive to music therapy; responsive to neurofeedback meditation; responsive to extradoses of antipsychotic; responsive to sleep aid; responsive to ECT; responsive to TMS; currently in active manic episode; currently in depressive episode; currently experiencing psychosis; currently in euthymic mood; currently in depressive mood; currently in elevated mood; or a combination thereof.

18. A device transmitting a medical subtype to a remote device, the device comprising:
a user terminal comprising a processor and memory, wherein the user terminal is configured to receive user input from a user;
a data collection unit configured to collect biometric data from a wearable device;
an assessment unit configured to:
process the biometric data to determine a medical subtype and store the determined medical subtype in memory,
dynamically update the stored medical subtype in response to receiving new biometric data that indicates either the stored medical subtype is incorrect or needs an additional qualifier; and
a medical subtype token generator that prepares a data packet that includes a representation for the stored medical subtype for transfer to the remote device; and
a medical subtype visual token renderer that prepares a visual image as a representation for the stored medical subtype for transfer to the remote device as medical subtype visual token (MSVT).

19. The device of claim 18, further comprising:
a camera configured to capture an image of a medical subtype visual token displaced on a screen of a remote device or printed-out on a paper a medical subtype token interpreter configured to convert the image of the medical subtype visual token into a data structure that stores the medical subtype; and
a wired or wireless port or a blue tooth port that receives a data packet that includes a representation of a medical subtype from a remote device.

20. The device of claim 18, further comprising:
an intervention manager comprising a processor, the intervention manager configured to offer the intervention to the user in response to the alert from the assessment unit,
wherein the intervention comprises at least one event selected from the group consisting of patient notification, healthcare provider notification, social support notification, suicide hotline connection, virtual therapy, music therapy, mindfulness therapy, light therapy, auditory hallucination avatar therapy, RX dispensing, biofeedback session, neurofeedback session, call an ambulance, or a combination thereof;
a screen configured to display the visual image of the medical subtype visual token in order to allow a camera installed or connected to the remote device to scan the medical subtype visual token to transfer the subtype information; and
a data transmitter configured to transmit the medical subtype token to the remote device via Internet, Bluetooth, hard wire, or a memory key.
